(19) United States
(12) Patent Application Publication
(10) Pub. No.: US 2008/0044390 A1
(43) Pub. Date: Feb. 21, 2008

(54) Methods and compositions for the treatment of neurodegenerative disorders

(76) Inventors: Xiaowei Jin, Cambridge, MA (US); Amy Beth Wilson, Allston, MA (US); Jane Staunton, Cambridge, MA (US); Douglas MacDonald, Los Angeles, CA (US)

Correspondence Address: CLARK & ELBING LLP, 101 FEDERAL STREET, BOSTON, MA, 02110, US

(21) Appl. No.: 11/891,552

(22) Filed: Aug. 10, 2007

Related U.S. Application Data
(60) Provisional application No. 60/837,448, filed on Aug. 11, 2006.
(60) Provisional application No. 60/898,479, filed on Jan. 31, 2007.
(60) Provisional application No. 60/925,777, filed on Apr. 23, 2007.
(60) Provisional application No. 60/958,832, filed on Jul. 9, 2007.

(51) Int. Cl. A61K 31/496; A61K 31/155; A61K 31/17; A61K 31/175; A61K 31/27; A61K 31/352; A61K 31/5415; A61K 31/56; A61K 31/70; C12Q 1/68; A61P 25/00; A61K 45/00; A61K 31/675; A61K 31/551; A61K 31/421; A61K 31/426; A61K 31/444; A61K 31/445; A61K 31/47

(52) U.S. Cl. 424/93.7; 435/6; 514/173; 514/178; 514/182; 514/220; 514/226.2; 514/253.05; 514/253.09; 514/291; 514/313; 514/327; 514/335; 514/35; 514/370; 514/376; 514/43; 514/456; 514/478; 514/588; 514/592; 514/636; 514/81

(57) ABSTRACT

The present invention features compositions, kits, and methods for treating, preventing, and ameliorating neurodegenerative disorders, e.g., Huntington's disease.

### CROSS REFERENCE TO RELATED APPLICATIONS

This application claims priority from U.S. Provisional Application No. 60/837,448, filed Aug. 11, 2006, U.S. Provisional Application No. 60/898,479, filed Jan. 31, 2007, U.S. Provisional Application No. 60/925,777, filed Apr. 23, 2007, and U.S. Provisional Application No. 60/958,832, filed Jul. 9, 2007, each of which is hereby incorporated by reference.

### BACKGROUND OF THE INVENTION

In general, this invention relates to the treatment, prevention, and amelioration of neurodegenerative disorders, e.g., Huntington's disease, and symptoms thereof.

Neurodegenerative disorders affect millions of individuals. One class of these disorders, the polyglutamine expansion disorders, is characterized by the presence of an expanded CAG repeat region within the coding sequence of a gene. While the threshold length of the CAG expansion is variable in these disorders, longer repeat length generally results in an earlier onset of the disease. For Huntington's disease, the threshold CAG repeat length for the onset of the disease is generally regarded as greater than 38 CAGs, resulting in a polyglutamine domain proximal to the N-terminus of the Huntingtin protein.

Huntington's disease (HD), one of at least nine known inherited polyglutamine expansion disorders, affects men and women with equal frequency, about 5-10 per 100,000. It can be characterized by five hallmark features: heritability; chorea; behavioral or psychiatric disturbances; cognitive impairment (dementia); and late-onset, with death occurring 15-20 years post-onset of motor dysfunctions. In most patients, the onset of the disease occurs in the third to fifth decade of life.

HD is an autosomal dominant disorder with a gene mutation on chromosome 4. This gene encodes a large protein, huntingtin (Htt), with multiple important functions. HD is caused by an expansion of the CAG repeat in the huntingtin (htt) gene, resulting in an expanded polyglutamine (poly Q) region near the N-terminus of Htt. Although the disease progression in HD is accompanied by widespread loss of neurons in the brain, the pathology is seen earliest in the striatum, in particular the medium spiny neurons, and to a lesser extent in the cerebral cortex.

The pathologic mechanisms underlying HD are not yet completely understood. Leading hypotheses include excitotoxicity, mitochondrial dysfunction, deficiencies in ubiquitin-mediated proteolysis, protease-dependent accumulation of poly-glutamine protein fragments, formation of cytosolic and nuclear inclusions, changes in gene expression, and neuronal cell degeneration and death. Although the mode of neuronal cell death continues to be debated, considerable evidence suggests that apoptosis plays an important role.

There are no current HD therapies, although some patients treat their symptoms with conventional neuroleptics to decrease chorea, and psychotropic medications to address depression, obsessive compulsive symptoms, or psychosis. There are currently no effective therapies for preventing the onset or slowing the progression of HD. Thus, there is a need to develop effective new therapies for treating, preventing, or ameliorating HD and other neurodegenerative disorders.

### SUMMARY OF THE INVENTION

The present invention features compositions, methods, and kits for treating, preventing, and ameliorating neurodegenerative disorders. The compositions, methods, and kits of the present invention may be particularly useful for treating patients having or at risk of having a polyglutamine expansion disorder, such as HD. The compositions, methods, and kits of the present invention also may be useful for treating symptoms or complications associated with neurodegenerative disorders, e.g., chorea, depression, obsessive-compulsive behavior, psychosis, dystonia (e.g., jaw clenching), and behavioral disturbances.

Accordingly, the invention features, in one instance, a composition that includes one or more first agents selected independently from any of the agents of Tables 1a and 1b, and one or more second, different agents selected independently from any one of the classes or agents of Tables 1a, 1b, and 2. The agent or agents of Tables 1a and 1b can be, e.g., GSK-3β inhibitors, CDK inhibitors, PKR inhibitors, EGFR inhibitors, flavonoids, antioxidants, PDE inhibitors, or caspase inhibitors. In certain embodiments, the first and second agents are selected from a single row of Table 3. In some instances, the first agent and second agent are present in amounts that, when administered to a patient, are sufficient to treat, prevent, or ameliorate a neurodegenerative disorder, e.g., a disorder selected from the group consisting of a polyglutamine expansion disorder (e.g., HD, dentatorubropallidoluysian atrophy, Kennedy's disease (also referred to as spinobulbar muscular atrophy), and spinocerebellar ataxia (e.g., type 1, type 2, type 3 (also referred to as Machado-Joseph disease), type 6, type 7, and type 17)), another trinucleotide repeat expansion disorder (e.g., fragile X syndrome, fragile XE mental retardation, Friedreich's ataxia, myotonic dystrophy, spinocerebellar ataxia type 8, and spinocerebellar ataxia type 12), Alexander disease, Alper's disease, Alzheimer disease, amyotrophic lateral sclerosis, ataxia telangiectasia, Batten disease (also referred to as Spielmeyer-Vogt-Sjogren-Batten disease), Canavan disease, Cockayne syndrome, corticobasal degeneration, Creutzfeldt-Jakob disease, ischemia stroke, Krabbe disease, Lewy body dementia, multiple sclerosis, multiple system atrophy, Parkinson's disease, Pelizaeus-Merzbacher disease, Pick's disease, primary lateral sclerosis, Refsum's disease, Sandhoff disease, Schilder's disease, spinal cord injury, spinal muscular atrophy, Steele-Richardson-Olszewski disease, and Tabes dorsalis. The composition may be formulated for any route of administration, e.g., oral, systemic, intracranial, intrathecal, or epidural administration.

The invention also features a method for treating, preventing, or ameliorating a neurodegenerative disorder by administering to a patient one or more agents selected independently from any of the agents of Tables 1a and 1b in an amount sufficient to treat, prevent, or ameliorate the neurodegenerative disorder.

The invention further features a method for treating, preventing, or ameliorating a neurodegenerative disorder by administering to a patient one or more agents selected independently from any of the agents of Table 1b and one or more different agents selected independently from any one of the classes or agents of Table 2 in amounts sufficient to treat, prevent, or ameliorate the neurodegenerative disorder.

The invention additionally features a method for treating, preventing, or ameliorating a neurodegenerative disorder by administering to a patient at least two different agents selected independently from any of the agents of Tables 1a and 1b, wherein the first and second agents are administered simultaneously or within 28 days of each other, in amounts that together are sufficient to treat, prevent, or ameliorate the neurodegenerative disorder. In certain embodiments, the first and second agents are selected from a single row of Table 3. The first and second agents may be administered simultaneously or within one hour, two hours, four hours, six hours, 10 hours, 12 hours, 18 hours, 24 hours, three days, seven days, or 14 days of each other.

In some instances, in the methods described herein, the agent or agents administered to the patient, e.g., GSK-3β inhibitors, CDK inhibitors, PKR inhibitors, EGFR inhibitors, flavonoids, antioxidants, PDE inhibitors, or caspase inhibitors, may reduce the rate of neuronal death in the patient (e.g., a human) relative to the rate of neuronal death in a control. In addition, the methods may include an additional therapeutic regimen. For example, the additional therapeutic regimen may include administering to the patient an additional therapeutic agent, such that the agent or agents from Tables 1a and 1b and the additional therapeutic agent are present in amounts that, when administered to the patient, are sufficient to treat, prevent, or ameliorate a neurodegenerative disorder. The additional therapeutic agent may be, e.g., selected from any one of the classes or agents of Table 2. The agent or agents from Tables 1a or 1b and the additional therapeutic agent may be administered simultaneously or within one hour, two hours, four hours, six hours, 10 hours, 12 hours, 18 hours, 24 hours, three days, seven days, or 14 days of each other, via any route of administration.

The invention further features a kit that includes any one of the agents of Tables 1a and 1b and instructions for administering the agent to a patient having or at risk of having a neurodegenerative disorder. Optionally, the kit includes two, three, four, or more than four agents selected independently from any of the agents of Tables 1a and 1b that may, but need not be, admixed in the same composition. This kit may also include instructions for administering the additional agent or agents, or the admixed composition, to the patient.

The invention also features a kit that includes one, two, three, four, or more than four agents selected independently from any of the agents of Tables 1a and 1b and one or more agents selected independently from any one of the classes or agents of Table 2. The agents may, but need not, be admixed in the same composition. The kit also includes instructions for administering these agents to a patient having or at risk of having a neurodegenerative disorder.

The invention further features a kit that includes either one, two, three, four, or more than four agents selected independently from any of the agents of Tables 1a and 1b, or one or more agents selected independently from any one of the classes or agents of Table 2. The kit also includes instructions for administering these agents together to a patient having or at risk of having a neurodegenerative disorder.

In any of the kits of the invention described herein, two agents may be selected from a single row of Table 3.

In another instance, the invention features a method of identifying a combination that may be useful for the treatment, prevention, or amelioration of a neurodegenerative disorder, including the steps of: (a) providing cells that include a gene encoding a polyglutamine repeat polypeptide, such that the polypeptide includes an expanded polyglutamine repeat region relative to a wild-type polyglutamine repeat polypeptide; (b) inducing expression of the gene; (c) contacting the cells with an agent selected from any one of the agents of Tables 1a and 1b and a candidate compound; and (d) determining whether the combination of the agent and the candidate compound reduces cell death relative to cells contacted with the agent but not contacted with the candidate compound, wherein a reduction in cell death (as determined, e.g., by monitoring intracellular ATP levels) identifies the combination as a combination that may be useful for the treatment, prevention, or amelioration of a neurodegenerative disorder. The polyglutamine repeat polypeptide that includes the expanded polyglutamine repeat region may include, e.g., HttN90Q103, or another variant of a polypeptide associated with a polyglutamine expansion disorder. The method may use, e.g., mammalian cells, such as rat pheochromocytoma PC12 cells.

TABLE 1a

| | Max Effect | EC50 | |
|---|---|---|---|
| Agent | (% rescue) | (μM) | Assay |
| 1-(5-isoquinolinesulfonyl)-2-methylpiperazine | 28% | 15.5 | ATPlite |
| 5,6-dichloro-1-β-D-ribofuranosylbenzimidazole | 26% | 5.1 | ATPlite |
| 5-methyl-5-6-7-8-tetrahydropteroylglutamic-acid | 34% | 9.3 | CellTiter-Blue |
| A-134974 (e.g., dihydrochloride monohydrate) | 22% | 13.3 | ATPlite |
| Acetohexamide | 18% | 3.9 | CellTiter-Blue |
| Amlexanox | 18% | 5.7 | ATPlite |
| Amodiaquine | 62% | 12.3 | ATPlite |
| Androstanolone | 15% | 8.0 | ATPlite |
| Benzohydroxamic acid | 15% | 6.9 | ATPlite |
| BML-248 | 24% | 7.1 | ATPlite |
| Bucladesine | 18% | 19.6 | ATPlite |
| Carbachol (e.g., hydrochloride salt) | 15% | 6.8 | CellTiter-Blue |
| Chenodeoxycholic acid diacetate methyl ester | 24% | 2.1 | CellTiter-Blue |
| Chlorzoxazone | 14% | 15.1 | ATPlite |
| Chrysin | 14% | 1.1 | CellTiter-Blue |
| Clorophene | 30% | 0.9 | CellTiter-Blue |
| Diminazene (e.g., aceturate salt) | 35% | 5.6 | ATPlite |
| Heat shock protein inhibitor I (KNK-437) | 20% | 8.2 | CellTiter-Blue |
| Imidocarb (e.g., dipropionate salt) | 24% | 15.9 | ATPlite |
| Kaempferol | 15% | 12.2 | ATPlite |
| Maduramicin NH₄ | 19% | 0.04 | ATPlite |
| Methylglyoxal bis(guanylhydrazone) (e.g., | 79% | 7.7 | ATPlite |
| dihydrochloride hydrate salt) | | | |
| Narasin | 15% | 0.1 | ATPlite |
| Nigericin | 36% | ND | ATPlite |
| NKH-477 | 69% | 7.2 | CellTiter-Blue |
| Pefabloc SC | 34% | 6.8 | CellTiter-Blue |
| PKR inhibitor | 124% | 1.2 | ATPlite |
| Pregnenolone | 22% | 11.9 | CellTiter-Blue |
| Pyritinol | 17% | 15.1 | ATPlite |
| Salinomycin (e.g., sodium salt) | 24% | 0.2 | ATPlite |
| Spironolactone | 22% | 9.1 | ATPlite |
| Testolactone | 52% | 7.6 | CellTiter-Blue |
| Tetrahydropapaveroline (e.g., bromide salt) | 47% | 20.1 | ATPlite |
| Tosufloxacin (e.g., tosylate salt) | 20% | 9.9 | ATPlite |
| Tyrphostin 46 | 23% | 23.5 | ATPlite |
| Vanadyl (e.g., sulfate hydrate salt) | 19% | 0.6 | ATPlite |
| Zopiclone | 25% | 6.7 | ATPlite |

TABLE 1b

| | Max | EC50 | |
|---|---|---|---|
| Agent | Effect (% rescue) | (μM) | Assay |
| Aicar | 23% | 14.1 | CellTiter-Blue |
| Alsterpaullone | 64% | 1.8 | ATPlite |
| Calcitriol | 33% | 0.01 | CellTiter-Blue |
| Caspase inhibitor III (BOC-D-fmk) | 119% | 3.9 | ATPlite |
| Celastrol | 19% | 0.3 | ATPlite |
| Chloroquine (e.g., phosphate salt) | 16% | 9.7 | ATPlite |
| Dehydroepiandrosterone | 21% | 11.0 | CellTiter-Blue |
| Diphenyleneiodonium (e.g., chloride salt) | 26% | 0.1 | CellTiter-Blue |
| Digitoxin | 11% | 3.5 | CellTiter-Blue |
| Divalproex (e.g., sodium salt) | 19% | 15.2 | ATPlite |
| Exemestane | 16% | 1.5 | CellTiter-Blue |
| Fasudil | 26% | 9.9 | ATPlite |
| Fisetin | 52% | 12.4 | ATPlite |
| Forskolin | 18% | 0.2 | ATPlite |
| GSK-3β inhibitor VIII (AR-A014418) | 43% | 3.6 | CellTiter-Blue |
| Hydroxychloroquine (e.g., sulfate salt) | 29% | 5.2 | ATPlite |
| Hydroxyurea | 22% | 7.5 | ATPlite |
| Indirubin-3′-monooxime | 30% | 2.4 | ATPlite |
| Isoliquiritigenin | 27% | 12.5 | ATPlite |
| Kenpaullone | 87% | 4.7 | ATPlite |
| Mofebutazone | 19% | 5.6 | ATPlite |
| Novobiocin (e.g., sodium salt) | 27% | 3.0 | CellTiter-Blue |
| Pifithrin α | 15% | 6.0 | CellTiter-Blue |
| Pyruvate (e.g., sodium salt) | 5% | 1.2 | ATPlite |
| Q-VD-OPH | 72% | 0.6 | ATPlite |
| Quercetin | 23% | 16.4 | ATPlite |
| Rosiglitazone (e.g., maleate salt) | 13% | 8.5 | ATPlite |
| SB-415286 | 52% | 12.9 | ATPlite |
| SU9516 | 66% | 3.3 | ATPlite |
| Tacrine (e.g., hydrochloride salt) | 15% | 39.6 | ATPlite |
| Tacrolimus (FK-506) | 36% | 0.1 | CellTiter-Blue |
| TPEN | 15% | 3.0 | ATPlite |
| Tranylcypromine (e.g., hemisulfate salt) | 16% | 2.2 | ATPlite |
| Troglitazone | 49% | 17.8 | CellTiter-Blue |
| UCH-L1 inhibitor | 21% | 0.5 | ATPlite |

TABLE 2

| Class | Exemplary agents |
|---|---|
| Antiapoptotic | Minocycline, troglitazone, pioglitazone, and taurosodeoxycholic |
| | acid (bile acid) |
| Antidepressant | Fluoxetine, sertraline hydrochloride, and nortriptyline |
| Antioxidant | Lipoic acid, melatonin, BN 8251, OPC-14117, and ascorbate |
| Antipsychotic/psychotropic | Haloperidol, clozapine, chlorpromazine, and olanzapine |
| Bioenergetic | Coenzyme Q10/idebenone, creatine, and dichloroacetate |
| COX inhibitor/NSAID | Flurbiprofen, naproxen sodium, diclofenac sodium, diclofenac |
| | potassium, aspirin, sulindac, diflunisal, piroxicam, indomethacin, |
| | ibuprofen, nabumetone, choline magnesium trisalicylate, sodium |
| | salicylate, salicylsalicylic acid, fenoprofen, ketoprofen, |
| | meclofenamate sodium, meloxicam, oxaprozin, sulindac, tolmetin, |
| | rofecoxib, celecoxib, valdecoxib, and lumiracoxib |
| Dopamine antagonist | Olanzapine, quetiapine, and tetrabenazine |
| Glutamate antagonist | Riluzole, remacemide, amantadine, memantine, ifendprodil, and |
| | eliprodil |
| Histone deacetylase inhibitor/ | Sodium butyrate, phenylbutyrate, suberoylanilide hydroxamic acid, |
| transcription regulator | and mithramycin |
| Heat shock protein regulator | Geldanamycin, celastrol, bimoclomol, and arimoclomol |
| Immune modulator | Copolymer 1 |
| Mood stabilizer | Lithium, valproate, and carbamazepine |
| Neuroleptic | Dopamine receptor blockers (e.g., haloperidol and perphenazine) |
| | and presynaptic dopamine depletors (e.g., reserpine) |
| Protein aggregation inhibitor | Cystamine and trehalose |
| Tranquilizer | Clonazepam, benzodiazepines (e.g., alprazolam, bromazepam, |
| | chlordiazepoxide, clobazam, clonazepam, diazepam, flunitrazepam, |
| | lorazepam, nitrazepam, oxazepam, temazepam, or triazolam), |
| | paroxetine, venlafaxine, and beta-blockers (e.g., acebutolol, atenolol, |
| | betaxolol, bisoprolol, carteolol, carvedilol, labetalol, metoprolol, |
| | nadolol, penbutolol, propranolol, sotalol, and timolol) |
| Trophic/restorative | GDNF, BDNF, CNTF, fetal striatal cells, and other cells for |
| | transplantation |
| Other | Cannabinoids (e.g., delta-9-tetrahydrocannabinol, |
| | tetrahydrocannabivarin, cannabidiol, cannabinol, cannabivarin, |
| | cannabidivarin, and cannabinolic acid), BCTC, lithium, ethyl-EPA, |
| | free fatty acids (e.g., palmitic acid, stearic acid, and arachidonic |
| | acid), rapamycin, KW6002, and botulinum toxin |

TABLE 3

| | Synergy | ADD |
|---|---|---|
| Combination Name | Score | Volume |
| NKH-477 × PKR inhibitor | 4.33 | 12.1 |
| NKH-477 × Q-VD-OPH | 6.25 | 7.21 |
| NKH-477 × Alsterpaullone | 4.14 | 11.3 |
| Amodiaquine × Imidocarb Dipropionate | 2.19 | 6.82 |
| Alsterpaullone × Amodiaquine | 3.32 | 6.58 |
| NKH-477 × SU9516 | 1.21 | 6.57 |
| PKR inhibitor × Quercetin | 1.86 | 5.56 |
| PKR inhibitor × UCH-L1 inhibitor | 0.94 | 4.64 |
| PKR inhibitor × Tyrphostin 46 | 1.64 | 4.6 |
| Alsterpaullone × diminazene aceturate | 1.26 | 3.88 |
| PKR inhibitor × diminazene aceturate | 1.48 | 3.74 |
| Amodiaquine × diminazene aceturate | 1.05 | 3.71 |
| PKR inhibitor × Tacrolimus (FK-506) | 1.26 | 3.58 |
| Alsterpaullone × Fisetin | 1.27 | 3.56 |
| Forskolin × PKR inhibitor | 1.34 | 3.49 |
| Amodiaquine × Caspase Inhibitor III | 1.65 | 3.45 |
| Amodiaquine × Q-VD-OPH | 3.29 | 1.79 |
| NKH-477 × Hydroxychloroquine | 0.32 | 3.36 |
| Kaempferol × PKR inhibitor | 0.98 | 3.27 |
| Alsterpaullone × BML-248 | 0.95 | 3.15 |
| Celastrol × Clorofene | 0.92 | 3.02 |
| Calcitriol × PKR inhibitor | 0.44 | 3.00 |
| Alsterpaullone × Tyrphostin 46 | 0.85 | 2.91 |
| Hydroxychloroquine Sulfate × Testolactone | 0.47 | 2.85 |
| Amodiaquine × Tyrphostin 46 | 0.83 | 2.84 |
| Alsterpaullone × PKR inhibitor | 1.30 | 2.78 |
| Amodiaquine × Kenpaullone | 0.89 | 2.72 |
| Kenpaullone × diminazene aceturate | 0.40 | 2.71 |
| NKH-477 × Quercetin | 0.22 | 2.61 |
| Alsterpaullone × Quercetin | 0.66 | 2.67 |
| 1-(5-Isoquinolinesulfonyl)-2-Methylpiperazine × Alsterpaullone | 0.56 | 2.50 |
| Amodiaquine × Vanadyl Sulfate Hydrate | 1.27 | 2.45 |
| Amodiaquine × Tosufloxacin tosylate | 0.70 | 2.35 |
| Alsterpaullone × Dehydroepiandrosterone | 0.55 | 2.33 |
| Hydroxychloroquine Sulfate × diminazene aceturate | 0.38 | 2.33 |
| Alsterpaullone × Hydroxychloroquine Sulfate | 0.74 | 2.31 |
| Alsterpaullone × Fasudil | 0.59 | 2.25 |
| Alsterpaullone × Digitoxin | 0.59 | 2.23 |
| Amodiaquine × Tacrolimus (FK-506) | 1.14 | 2.19 |
| Quercetin × diminazene aceturate | 0.47 | 2.19 |
| Tacrine Hydrochloride × diminazene aceturate | 0.27 | 2.16 |
| Acetohexamide × Alsterpaullone | 0.62 | 2.12 |
| Quercetin × SU9516 | 0.46 | 2.09 |
| Amodiaquine × Testolactone | 0.66 | 2.08 |
| PKR inhibitor × Tosufloxacin tosylate | 0.43 | 2.07 |
| Amodiaquine × Indirubin-3′-Monooxime | 0.61 | 2.04 |
| Carbachol HCl × PKR inhibitor | 0.42 | 2.04 |
| PKR inhibitor × Testolactone | 0.49 | 2.04 |
| Amodiaquine × Isoliquiritigenin | 0.59 | 2.03 |
| Caspase Inhibitor III × Celastrol | 0.77 | 2.02 |
| Amodiaquine × UCH-L1 inhibitor | 1.02 | 1.96 |
| Alsterpaullone × Tosufloxacin tosylate | 0.54 | 1.96 |
| Fisetin × PKR inhibitor | 0.87 | 1.96 |
| PKR inhibitor × Vanadyl Sulfate Hydrate | 0.48 | 1.95 |
| BML-248 × PKR inhibitor | 0.54 | 1.95 |
| 5,6-Dichloro-1-Beta-D-Ribofuranosylbenzimidazole × Amodiaquine | 0.38 | 1.95 |
| Alsterpaullone × SU9516 | 0.55 | 1.94 |
| Exemestane × PKR inhibitor | 0.70 | 1.87 |
| NKH-477 × Fisetin | 0.27 | 1.86 |
| Amodiaquine × SU9516 | 0.35 | 1.80 |
| Alsterpaullone × Testolactone | 0.52 | 1.77 |
| Fisetin × diminazene aceturate | 0.34 | 1.76 |
| Fisetin × Kenpaullone | 0.33 | 1.73 |
| Caspase Inhibitor III × Heat Shock Protein Inhibitor I | 0.21 | 1.72 |
| Novobiocin Sodium × Quercetin | 0.22 | 1.72 |
| Imidocarb Dipropionate × PKR inhibitor | 0.57 | 1.72 |
| Alsterpaullone × Pyritinol | 0.53 | 1.68 |
| Chloroquine Phosphate × diminazene aceturate | 0.19 | 1.65 |
| Alsterpaullone × Isoliquiritigenin | 0.54 | 1.63 |
| Alsterpaullone × Indirubin-3′-Monooxime | 0.54 | 1.61 |
| Aicar × PKR inhibitor | 0.58 | 1.58 |
| Testolactone × diminazene aceturate | 0.26 | 1.58 |
| SU9516 × diminazene aceturate | 0.29 | 1.58 |
| Caspase Inhibitor III × Troglitazone | 0.31 | 1.57 |
| Amodiaquine × PKR inhibitor | 1.37 | 1.55 |
| Tranylcypromine Hemisulfate × Tyrphostin 46 | 0.17 | 1.54 |
| Hydroxychloroquine Sulfate × Isoliquiritigenin | 0.20 | 1.52 |
| Alsterpaullone × Chlorzoxazone | 0.55 | 1.46 |
| Caspase Inhibitor III × Clorofene | 0.20 | 1.46 |
| SU9516 × Tyrphostin 46 | 0.20 | 1.45 |
| Caspase Inhibitor III × Hydroxychloroquine Sulfate | 0.30 | 1.44 |
| Q-VD-OPH × Hydroxychloroquine Sulfate | 1.25 | 3.07 |
| Quercetin × Rosiglitazone Maleate | 0.22 | 1.43 |
| Amodiaquine × SB 415286 | 0.32 | 1.42 |
| Alsterpaullone × Caspase Inhibitor III | 0.67 | 1.41 |
| Chlorzoxazone × PKR inhibitor | 0.21 | 1.41 |
| 5,6-Dichloro-1-Beta-D-Ribofuranosylbenzimidazole × PKR inhibitor | 0.31 | 1.40 |
| Novobiocin Sodium × PKR inhibitor | 0.48 | 1.40 |
| Clorofene × PKR inhibitor | 0.33 | 1.39 |
| Androstanolone × diminazene aceturate | 0.15 | 1.37 |
| Quercetin × Tyrphostin 46 | 0.19 | 1.37 |
| 5,6-Dichloro-1-Beta-D-Ribofuranosylbenzimidazole × Hydroxychloroquine | 0.12 | 1.37 |
| Sulfate | | |
| PKR inhibitor × Pyritinol | 0.32 | 1.37 |
| Tyrphostin 46 × diminazene aceturate | 0.20 | 1.37 |
| NKH-477 × Amodiaquine | 0.18 | 1.37 |
| Chloroquine Phosphate × Isoliquiritigenin | 0.14 | 1.34 |
| Forskolin × Indirubin-3′-Monooxime | 0.11 | 1.34 |
| Amodiaquine × Chrysin | 0.25 | 1.32 |
| Imidocarb Dipropionate × SU9516 | 0.11 | 1.32 |
| Carbachol Hcl × Caspase Inhibitor III | 0.13 | 1.32 |
| Forskolin × Hydroxychloroquine Sulfate | 0.14 | 1.3 |
| Alsterpaullone × Spironolactone | 0.25 | 1.3 |
| Amodiaquine × Forskolin | 0.22 | 1.3 |
| Isoliquiritigenin × SU9516 | 0.18 | 1.26 |
| Hydroxychloroquine Sulfate × PKR inhibitor | 1.02 | 1.26 |
| A-134974, Dihydrochloride; monohydrate × PKR inhibitor | 0.18 | 1.24 |
| Caspase Inhibitor III × Fisetin | 0.32 | 1.23 |
| Heat Shock Protein Inhibitor I × SU9516 | 0.15 | 1.23 |
| Isoliquiritigenin × PKR inhibitor | 0.40 | 1.23 |
| Acetohexamide × Amodiaquine | 0.23 | 1.22 |
| Amodiaquine × Zopiclone | 0.26 | 1.22 |
| Caspase Inhibitor III × TPEN | 0.29 | 1.22 |
| PKR inhibitor × SU9516 | 0.57 | 1.21 |
| Hydroxychloroquine Sulfate × Tacrolimus (FK-506) | 0.16 | 1.21 |
| Amodiaquine × TPEN | 0.72 | 1.17 |
| Fisetin × Troglitazone | 0.16 | 1.17 |
| Chrysin × Hydroxychloroquine Sulfate | 0.13 | 1.16 |
| Fisetin × Tyrphostin 46 | 0.16 | 1.14 |
| Kenpaullone × PKR inhibitor | 0.44 | 1.13 |
| Amodiaquine × Quercetin | 0.29 | 1.12 |
| 1-(5-Isoquinolinesulfonyl)-2-Methylpiperazine × Caspase Inhibitor III | 0.20 | 1.12 |
| Alsterpaullone × Tetrahydropapaveroline Hydrobromide | 0.68 | 1.1 |
| Androstanolone × Tyrphostin 46 | 0.11 | 1.1 |
| Fisetin × Indirubin-3′-Monooxime | 0.22 | 1.08 |
| 1-(5-Isoquinolinesulfonyl)-2-Methylpiperazine × PKR inhibitor | 0.65 | 1.08 |
| 1-(5-Isoquinolinesulfonyl)-2-Methylpiperazine × Amodiaquine | 0.28 | 1.07 |
| Androstanolone × Quercetin | 0.13 | 1.07 |
| Celastrol × Heat Shock Protein Inhibitor I | 0.37 | 1.07 |
| PKR inhibitor × Sodium Pyruvate | 0.33 | 1.06 |
| Caspase Inhibitor III × Tyrphostin 46 | 0.18 | 1.05 |
| Hydroxychloroquine Sulfate × Tosufloxacin tosylate | 0.10 | 1.03 |
| Amodiaquine × Hydroxyurea | 0.33 | 1.03 |
| Exemestane × Hydroxychloroquine Sulfate | 0.11 | 1.02 |
| Chloroquine Phosphate × Testolactone | 0.13 | 1.02 |
| Fisetin × SU9516 | 0.23 | 1.01 |
| Divalproex Sodium × PKR inhibitor | 0.31 | 1.01 |
| Amodiaquine × Tranylcypromine Hemisulfate | 0.39 | 1.00 |
| Quercetin × Tacrine Hydrochloride | 0.10 | 0.99 |
| Fasudil × PKR inhibitor | 0.32 | 0.99 |
| PKR inhibitor × TPEN | 0.38 | 0.99 |
| Caspase Inhibitor III × Tetrahydropapaveroline Hydrobromide | 0.71 | 0.98 |
| Fisetin × Novobiocin Sodium | 0.19 | 0.98 |
| Caspase Inhibitor III × Divalproex Sodium | 0.21 | 0.96 |
| Tosufloxacin tosylate × Tyrphostin 46 | 0.11 | 0.96 |
| Hydroxychloroquine Sulfate × Imidocarb Dipropionate | 0.20 | 0.95 |
| 1-(5-Isoquinolinesulfonyl)-2-Methylpiperazine × Hydroxychloroquine Sulfate | 0.11 | 0.95 |
| Tranylcypromine Hemisulfate × diminazene aceturate | 0.13 | 0.90 |
| Caspase Inhibitor III × Chloroquine Phosphate | 0.20 | 0.85 |
| Caspase Inhibitor III × Tacrolimus (FK-506) | 0.16 | 0.84 |
| GSK-3B Inhibitor VIII × PKR inhibitor | 0.19 | 0.83 |
| PKR inhibitor × Tacrine Hydrochloride | 0.18 | 0.82 |
| Exemestane × Quercetin | 0.08 | 0.80 |
| PKR inhibitor × Tetrahydropapaveroline Hydrobromide | 0.78 | 0.78 |
| BML-248 × Caspase Inhibitor III | 0.20 | 0.76 |
| Amodiaquine × Spironolactone | 0.29 | 0.76 |
| Acetohexamide × Caspase Inhibitor III | 0.13 | 0.71 |
| Amodiaquine × Hydroxychloroquine Sulfate | 0.26 | 0.70 |
| Fisetin × Testolactone | 0.11 | 0.69 |
| Quercetin × Tosufloxacin tosylate | 0.09 | 0.66 |
| Amodiaquine × Exemestane | 0.21 | 0.66 |
| Chloroquine Phosphate × PKR inhibitor | 0.26 | 0.60 |
| Spironolactone × diminazene aceturate | 0.12 | 0.59 |
| Hydroxychloroquine Sulfate × Tyrphostin 46 | 0.10 | 0.59 |
| Caspase Inhibitor III × Rosiglitazone Maleate | 0.13 | 0.58 |
| Fisetin × Tosufloxacin tosylate | 0.16 | 0.56 |
| Isoliquiritigenin × diminazene aceturate | 0.10 | 0.56 |
| Exemestane × Kenpaullone | 0.05 | 0.55 |
| Testolactone × Tyrphostin 46 | 0.10 | 0.55 |
| BML-248 × Fisetin | 0.14 | 0.54 |
| Quercetin × Testolactone | 0.10 | 0.54 |
| Amodiaquine × Fisetin | 0.37 | 0.53 |
| Fisetin × Hydroxychloroquine Sulfate | 0.19 | 0.50 |
| Fisetin × Rosiglitazone Maleate | 0.07 | 0.50 |
| Chloroquine Phosphate × Fisetin | 0.08 | 0.48 |
| A-134974, Dihydrochloride; monohydrate × Fisetin | 0.08 | 0.43 |
| Isoliquiritigenin × Kenpaullone | 0.08 | 0.42 |
| 5,6-Dichloro-1-Beta-D-Ribofuranosylbenzimidazole × Fisetin | 0.13 | 0.41 |
| Rosiglitazone Maleate × diminazene aceturate | 0.07 | 0.32 |
| Kenpaullone × Tetrahydropapaveroline Hydrobromide | 0.29 | 0.32 |
| Tetrahydropapaveroline Hydrobromide × diminazene aceturate | 0.16 | 0.32 |
| Isoliquiritigenin × Quercetin | 0.10 | 0.29 |
| Indirubin-3′-Monooxime × Isoliquiritigenin | 0.05 | 0.27 |
| GSK-3B Inhibitor VIII × Hydroxyurea | 0.05 | 0.25 |
| 1-(5-Isoquinolinesulfonyl)-2-Methylpiperazine × diminazene aceturate | 0.16 | 0.25 |
| Celastrol × PKR inhibitor | 0.53 | 0.24 |
| Fisetin × Pyritinol | 0.07 | 0.22 |
| Quercetin × TPEN | 0.10 | 0.18 |
| Forskolin × Isoliquiritigenin | 0.08 | 0.18 |
| Caspase Inhibitor III × Quercetin | 0.07 | 0.15 |
| 1-(5-Isoquinolinesulfonyl)-2-Methylpiperazine × Fisetin | 0.09 | 0.14 |
| Pyritinol × Quercetin | 0.07 | 0.06 |
| Exemestane × Fisetin | 0.14 | 0.04 |

By “an amount sufficient” is meant the amount of a compound, alone or in combination with another therapeutic regimen, required to treat, prevent, or ameliorate a neurodegenerative disorder, such as HD, in a clinically relevant manner. A sufficient amount of an active compound used to practice the present invention for therapeutic treatment of neurodegenerative disorders varies depending upon the manner of administration, age, and general health of the patient. Ultimately, the prescribers will decide the appropriate amount and dosage regimen. Additionally, an effective amount may be an amount of compound in the combination of the invention that is safe and efficacious in the treatment of a patient having a neurodegenerative disorder, such as HD, over each agent alone as determined and approved by a regulatory authority (such as the U.S. Food and Drug Administration).

By “candidate compound” is meant a chemical, be it naturally-occurring or artificially-derived. Candidate compounds may include, for example, peptides, polypeptides, synthetic organic molecules, naturally occurring organic molecules, nucleic acid molecules, peptide nucleic acid molecules, and components and derivatives thereof.

Compounds that may be useful in the invention include those described herein in any of their pharmaceutically acceptable forms, including isomers such as diastereomers and enantiomers, salts, esters, solvates, and polymorphs thereof, as well as racemic mixtures and pure isomers of the compounds described herein.

Compounds useful in the invention may also be isotopically labeled compounds. Useful isotopes include hydrogen, carbon, nitrogen, oxygen, phosphorous, fluorine, and chlorine, (e.g., ²H, ³H, ¹³C, ¹⁴C, ¹⁵N, ¹⁸O, ¹⁷O, ³¹P, ³²P, ³⁵S, ¹⁸F, and ³⁶Cl). Isotopically-labeled compounds can be prepared by synthesizing a compound using a readily available isotopically-labeled reagent in place of a non-isotopically-labeled reagent.

By “expanded polyglutamine repeat region” is meant a region of a polyglutamine repeat polypeptide in which the number of glutamine residues is greater than the number of glutamine residues in a corresponding wild-type polypeptide. An exemplary polypeptide containing an expanded polyglutamine repeat region is, e.g., HttN90Q103, which contains a region of 103 glutamine residues. An expanded polyglutamine repeat region contains greater than, e.g., 5, 10, 15, 20, 25, 30, 35, 40, 45, 50, 55, 60, 65, 70, 75, 80, 85, 90, 95, or even 100 glutamine residues. Alternatively, an expanded polyglutamine repeat region contains greater than, e.g., 1, 2, 3, 4, 5, 10, 15, 20, 25, 30, 35, 40, 45, 50, 55, 60, 65, 70, 75, 80, 85, 90, 95, or even 100 more glutamine residues than the number of glutamine residues in a corresponding wild-type polypeptide.

By a “high dosage” is meant at least 5% more (e.g., at least 10%, 20%, 50%, 100%, 200%, or even 300%) than the highest standard recommended dosage of a particular compound formulated for a given route of administration for treatment of any human disease or condition. For example, a high dosage of an agent that prevents or slows the rate of neural deterioration or death associated with a neurodegenerative disorder and that is formulated for intravenous administration may differ from a high dosage of the same agent formulated for oral administration.

By a “low dosage” is meant at least 5% less (e.g., at least 10%, 20%, 50%, 80%, 90%, or even 95%) than the lowest standard recommended dosage of a particular compound formulated for a given route of administration for treatment of any human disease or condition.

By “neurodegenerative disorder” is meant any disease or disorder caused by or associated with the deterioration of cells or tissues of the nervous system. Exemplary neurodegenerative disorders are polyglutamine expansion disorders (e.g., HD, dentatorubropallidoluysian atrophy, Kennedy's disease (also referred to as spinobulbar muscular atrophy), and spinocerebellar ataxia (e.g., type 1, type 2, type 3 (also referred to as Machado-Joseph disease), type 6, type 7, and type 17)), other trinucleotide repeat expansion disorders (e.g., fragile X syndrome, fragile XE mental retardation, Friedreich's ataxia, myotonic dystrophy, spinocerebellar ataxia type 8, and spinocerebellar ataxia type 12), Alexander disease, Alper's disease, Alzheimer disease, amyotrophic lateral sclerosis, ataxia telangiectasia, Batten disease (also referred to as Spielmeyer-Vogt-Sjogren-Batten disease), Canavan disease, Cockayne syndrome, corticobasal degeneration, Creutzfeldt-Jakob disease, ischemia stroke, Krabbe disease, Lewy body dementia, multiple sclerosis, multiple system atrophy, Parkinson's disease, Pelizaeus-Merzbacher disease, Pick's disease, primary lateral sclerosis, Refsum's disease, Sandhoff disease, Schilder's disease, spinal cord injury, spinal muscular atrophy, Steele-Richardson-Olszewski disease, and Tabes dorsalis.

By “patient” is meant any animal, e.g., a mammal (e.g., a human). Other animals that can be treated using the methods, compositions, and kits of the invention include horses, dogs, cats, pigs, goats, rabbits, hamsters, monkeys, guinea pigs, rats, mice, lizards, snakes, sheep, cattle, fish, and birds.

A patient who is being treated for a neurodegenerative disorder, e.g., HD, is one who has been diagnosed by a medical practitioner as having such a condition. Diagnosis may be performed by any suitable means, such as those described herein. A patient in whom the development of a neurodegenerative disorder is being prevented may or may not have received such a diagnosis. One in the art will understand that patients of the invention may have been subjected to standard tests or may have been identified, without examination, as one at high risk due to the presence of one or more risk factors, such as age, family history of neurodegenerative disorders, and psychological or psychiatric profile.

By “polyglutamine repeat polypeptide” is meant any polypeptide containing at least five consecutive glutamine residues. Exemplary polyglutamine repeat polypeptides are those associated with polyglutamine expansion disorders (e.g., HD, dentatorubropallidoluysian atrophy, Kennedy's disease (also referred to as spinobulbar muscular atrophy), and spinocerebellar ataxia (e.g., type 1, type 2, type 3 (also referred to as Machado-Joseph disease), type 6, type 7, and type 17)). For example, Htt, which is associated with HD, is a polyglutamine repeat polypeptide.

The terms “polypeptide” and “peptide” are used interchangeably and refer to any chain of more than two natural or unnatural amino acids, regardless of post-translational modification (e.g., glycosylation or phosphorylation), constituting all or part of a naturally-occurring or non-naturally occurring polypeptide or peptide, as is described herein.

As used herein, a natural amino acid is a natural α-amino acid having the L-configuration, such as those normally occurring in natural proteins. Unnatural amino acid refers to an amino acid, which normally does not occur in proteins, e.g., an epimer of a natural α-amino acid having the L configuration, that is to say an amino acid having the unnatural D-configuration; or a (D,L)-isomeric mixture thereof; or a homologue of such an amino acid, for example, a α-amino acid, an α,α-disubstituted amino acid, or an α-amino acid wherein the amino acid side chain has been shortened by one or two methylene groups or lengthened to up to 10 carbon atoms, such as an α-amino alkanoic acid with 5 up to and including 10 carbon atoms in a linear chain, an unsubstituted or substituted aromatic (α-aryl or α-aryl lower alkyl), for example, a substituted phenylalanine or phenylglycine.

By “systemic administration” is meant any nondermal route of administration, and specifically excludes topical and transdermal routes of administration.

By “treating, preventing, or ameliorating a neurodegenerative disorder” is meant ameliorating such a condition before or after its onset. As compared with an equivalent untreated control, such amelioration or degree of prevention is at least 5%, 10%, 20%, 30%, 40%, 50%, 60%, 70%, 80%, 90%, 95%, or 100% as measured by any standard technique.

In the generic descriptions of compounds of this invention, the number of atoms of a particular type in a substituent group is generally given as a range, e.g., an alkyl group containing from 1 to 6 carbon atoms or C₁-C₆ alkyl. Reference to such a range is intended to include specific references to groups having each of the integer number of atoms within the specified range. For example, an alkyl group from 1 to 6 carbon atoms includes each of C₁, C₂, C₃, C₄, C₅, and C₆. A C₁-C₁₂ heteroalkyl, for example, includes from 1 to 12 carbon atoms in addition to one or more heteroatoms. Other numbers of atoms and other types of atoms may be indicated in a similar manner.

As used herein, the terms “alkyl” and the prefix “alk-” are inclusive of both straight chain and branched chain groups and of cyclic groups, i.e., cycloalkyl. Cyclic groups can be monocyclic or polycyclic and preferably have from 3 to 6 ring carbon atoms, inclusive. Exemplary cyclic groups include cyclopropyl, cyclobutyl, cyclopentyl, and cyclohexyl groups.

By “C₁-C₆ alkyl” is meant a branched or unbranched hydrocarbon group having from 1 to 6 carbon atoms. A C₁-C₆ alkyl group may be substituted or unsubstituted. Exemplary substituents include alkoxy, aryloxy, sulfhydryl, alkylthio, arylthio, halogen, hydroxyl, fluoroalkyl, perfluoroalkyl, amino, aminoalkyl, disubstituted amino, quaternary amino, hydroxyalkyl, carboxyalkyl, and carboxyl groups. C₁-C₆ alkyls include, without limitation, methyl, ethyl, n-propyl, isopropyl, cyclopropyl, cyclopropylmethyl, n-butyl, iso-butyl, sec-butyl, tert-butyl, and cyclobutyl.

By “C₂-C₆ alkenyl” is meant a branched or unbranched hydrocarbon group containing one or more double bonds and having from 2 to 6 carbon atoms. A C₂-C₆ alkenyl may optionally include monocyclic or polycyclic rings, in which each ring desirably has from three to six members. The C₂-C₆ alkenyl group may be substituted or unsubstituted. Exemplary substituents include alkoxy, aryloxy, sulfhydryl, alkylthio, arylthio, halogen, hydroxyl, fluoroalkyl, perfluoralkyl, amino, aminoalkyl, disubstituted amino, quaternary amino, hydroxyalkyl, carboxyalkyl, and carboxyl groups. C₂-C₆ alkenyls include, without limitation, vinyl, allyl, 2-cyclopropyl-1-ethenyl, 1-propenyl, 1-butenyl, 2-butenyl, 3-butenyl, 2-methyl-1-propenyl, and 2-methyl-2-propenyl.

By “C₂-C₆ alkynyl” is meant a branched or unbranched hydrocarbon group containing one or more triple bonds and having from 2 to 6 carbon atoms. A C₂-C₆ alkynyl may optionally include monocyclic, bicyclic, or tricyclic rings, in which each ring desirably has five or six members. The C₂-C₆ alkynyl group may be substituted or unsubstituted. Exemplary substituents include alkoxy, aryloxy, sulfhydryl, alkylthio, arylthio, halogen, hydroxy, fluoroalkyl, perfluoralkyl, amino, aminoalkyl, disubstituted amino, quaternary amino, hydroxyalkyl, carboxyalkyl, and carboxyl groups. C₂-C₆ alkynyls include, without limitation, ethynyl, 1-propynyl, 2-propynyl, 1-butynyl, 2-butynyl, and 3-butynyl.

By “C₂-C₆ heterocyclyl” is meant a stable 5- to 7-membered monocyclic or 7- to 14-membered bicyclic heterocyclic ring which is saturated partially unsaturated or unsaturated (aromatic), and which consists of 2 to 6 carbon atoms and 1, 2, 3 or 4 heteroatoms independently selected from N, O, and S and including any bicyclic group in which any of the above-defined heterocyclic rings is fused to a benzene ring. The heterocyclyl group may be substituted or unsubstituted. Exemplary substituents include alkoxy, aryloxy, sulfhydryl, alkylthio, arylthio, halogen, hydroxy, fluoroalkyl, perfluoralkyl, amino, aminoalkyl, disubstituted amino, quaternary amino, hydroxyalkyl, carboxyalkyl, and carboxyl groups. The nitrogen and sulfur heteroatoms may optionally be oxidized. The heterocyclic ring may be covalently attached via any heteroatom or carbon atom which results in a stable structure, e.g., an imidazolinyl ring may be linked at either of the ring-carbon atom positions or at the nitrogen atom. A nitrogen atom in the heterocycle may optionally be quaternized. Preferably when the total number of S and O atoms in the heterocycle exceeds 1, then these heteroatoms are not adjacent to one another. Heterocycles include, without limitation, 1H-indazole, 2-pyrrolidonyl, 2H,6H-1,5,2-dithiazinyl, 2H-pyrrolyl, 3H-indolyl, 4-piperidonyl, 4aH-carbazole, 4H-quinolizinyl, 6H-1,2,5-thiadiazinyl, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazalonyl, carbazolyl, 4aH-carbazolyl, b-carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinylperimidinyl, phenanthridinyl, phenanthrolinyl, phenarsazinyl, phenazinyl, phenothiazinyl, phenoxathiinyl, phenoxazinyl, phthalazinyl, piperazinyl, piperidinyl, pteridinyl, piperidonyl, 4-piperidonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, pyrrolyl, quinazolinyl, quinolinyl, 4H-quinolizinyl, quinoxalinyl, quinuclidinyl, carbolinyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, xanthenyl. Preferred 5 to 10 membered heterocycles include, but are not limited to, pyridinyl, pyrimidinyl, triazinyl, furanyl, thienyl, thiazolyl, pyrrolyl, pyrazolyl, imidazolyl, oxazolyl, isoxazolyl, tetrazolyl, benzofuranyl, benzothiofuranyl, indolyl, benzimidazolyl, 1H-indazolyl, oxazolidinyl, isoxazolidinyl, benzotriazolyl, benzisoxazolyl, oxindolyl, benzoxazolinyl, quinolinyl, and isoquinolinyl. Preferred 5 to 6 membered heterocycles include, without limitation, pyridinyl, pyrimidinyl, triazinyl, furanyl, thienyl, thiazolyl, pyrrolyl, piperazinyl, piperidinyl, pyrazolyl, imidazolyl, oxazolyl, isoxazolyl, and tetrazolyl.

By “C₆-C₁₂ aryl” is meant an aromatic group having a ring system comprised of carbon atoms with conjugated 71 electrons (e.g., phenyl). The aryl group has from 6 to 12 carbon atoms. Aryl groups may optionally include monocyclic, bicyclic, or tricyclic rings, in which each ring desirably has five or six members. The aryl group may be substituted or unsubstituted. Exemplary substituents include alkyl, hydroxy, alkoxy, aryloxy, sulfhydryl, alkylthio, arylthio, halogen, fluoroalkyl, carboxyl, hydroxyalkyl, carboxyalkyl, amino, aminoalkyl, monosubstituted amino, disubstituted amino, and quaternary amino groups.

By “C₇-C₁₄ alkaryl” is meant an alkyl substituted by an aryl group (e.g., benzyl, phenethyl, or 3,4-dichlorophenethyl) having from 7 to 14 carbon atoms.

By “C₃-C₁₀ alkheterocyclyl” is meant an alkyl substituted heterocyclic group having from 3 to 10 carbon atoms in addition to one or more heteroatoms (e.g., 3-furanylmethyl, 2-furanylmethyl, 3-tetrahydrofuranylmethyl, or 2-tetrahydrofuranylmethyl).

By “C₁-C₇ heteroalkyl” is meant a branched or unbranched alkyl, alkenyl, or alkynyl group having from 1 to 7 carbon atoms in addition to 1, 2, 3 or 4 heteroatoms independently selected from the group consisting of N, O, S, and P. Heteroalkyls include, without limitation, tertiary amines, secondary amines, ethers, thioethers, amides, thioamides, carbamates, thiocarbamates, hydrazones, imines, phosphodiesters, phosphoramidates, sulfonamides, and disulfides. A heteroalkyl may optionally include monocyclic, bicyclic, or tricyclic rings, in which each ring desirably has three to six members. The heteroalkyl group may be substituted or unsubstituted. Exemplary substituents include alkoxy, aryloxy, sulfhydryl, alkylthio, arylthio, halogen, hydroxyl, fluoroalkyl, perfluoralkyl, amino, aminoalkyl, disubstituted amino, quaternary amino, hydroxyalkyl, hydroxyalkyl, carboxyalkyl, and carboxyl groups. Examples of C₁-C₇ heteroalkyls include, without limitation, methoxymethyl and ethoxyethyl.

By “halogen” is meant bromine, chlorine, iodine, or fluorine.

By “fluoroalkyl” is meant an alkyl group that is substituted with a fluorine atom.

By “perfluoroalkyl” is meant an alkyl group consisting of only carbon and fluorine atoms.

By “carboxyalkyl” is meant a chemical moiety with the formula —(R)—COOH, wherein R is selected from C₁-C₇ alkyl, C₂-C₇ alkenyl, C₂-C₇ alkynyl, C₂-C₆ heterocyclyl, C₆-C₁₂ aryl, C₇-C₁₄ alkaryl, C₃-C₁₀ alkheterocyclyl, or C₁-C₇ heteroalkyl.

By “hydroxyalkyl” is meant a chemical moiety with the formula —(R)—OH, wherein R is selected from C₁-C₇ alkyl, C₂-C₇ alkenyl, C₂-C₇ alkynyl, C₂-C₆ heterocyclyl, C₆-C₁₂ aryl, C₇-C₁₄ alkaryl, C₃-C₁₀ alkheterocyclyl, or C₁-C₇ heteroalkyl.

By “alkoxy” is meant a chemical substituent of the formula —OR, wherein R is selected from C₁-C₇ alkyl, C₂-C₇ alkenyl, C₂-C₇ alkynyl, C₂-C₆ heterocyclyl, C₆-C₁₂ aryl, C₇-C₁₄ alkaryl, C₃-C₁₀ alkheterocyclyl, or C₁-C₇ heteroalkyl.

By “aryloxy” is meant a chemical substituent of the formula —OR, wherein R is a C₆-C₁₂ aryl group.

By “alkylthio” is meant a chemical substituent of the formula —SR, wherein R is selected from C₁-C₇ alkyl, C₂-C₇ alkenyl, C₂-C₇ alkynyl, C₂-C₆ heterocyclyl, C₆-C₁₂ aryl, C₇-C₁₄ alkaryl, C₃-C₁₀ alkheterocyclyl, or C₁-C₇ heteroalkyl.

By “arylthio” is meant a chemical substituent of the formula —SR, wherein R is a C₆-C₁₂ aryl group.

By “quaternary amino” is meant a chemical substituent of the formula —(R)—N(R′)(R″)(R′″)⁺, wherein R, R′, R″, and R′″ are each independently an alkyl, alkenyl, alkynyl, or aryl group. R may be an alkyl group linking the quaternary amino nitrogen atom, as a substituent, to another moiety. The nitrogen atom, N, is covalently attached to four carbon atoms of alkyl, heteroalkyl, heteroaryl, and/or aryl groups, resulting in a positive charge at the nitrogen atom.

Other features and advantages of the invention will be apparent from the detailed description and from the claims.

### BRIEF DESCRIPTION OF THE DRAWINGS

FIG. 1A is a graph showing dose response rescue by BOC-D-FMK. PC12/HttN90Q103 cells were induced by 250 nM tebufenozide (teb), and cell viability was monitored using the ATPlite™ assay. The test was run with five replicate plates for each set using the following conditions: 72-hour teb induction and 48-hour drug treatment. FIG. 1B is a graph showing response rescue by BOC-D-FMK with an additional set of five replicate plates as in FIG. 1A, with 72-hour teb induction and 68-hour drug treatment.

### DETAILED DESCRIPTION OF THE INVENTION

We have identified compounds that, alone or in combination, may be effective in the treatment, prevention, or amelioration of neurodegenerative disorders. The compositions, methods, and kits of the present invention may be particularly useful for treating patients having or at risk of having a polyglutamine expansion disorder, e.g., HD. Accordingly, a patient that has been diagnosed with or is at risk of having a neurodegenerative disorder is administered one, two, three, four, or more agents selected independently from any of the agents of Tables 1a and 1b. Optionally, analogs of these agents may be employed. In the case of a polyglutamine expansion disorder, for example, such administration may prevent or slow the rate of neural deterioration or death. The ability of the agent to prevent or slow the rate of neural deterioration or death may be attributed, for example, to its ability to inhibit the disease-causing activity of a mutant polyglutamine protein, e.g., Htt. Optionally, the patient may also receive other therapeutic regimens.

In one particular example, the patient being treated is administered two agents selected independently from any of the agents of Tables 1a and 1b within 28 days of each other in amounts that together are sufficient to treat, prevent, or ameliorate the neurodegenerative disorder. The two agents are desirably administered within 14 days of each other, more desirably within seven days of each other, and even more desirably within twenty-four hours of each other, or even simultaneously. If desired, either one of the two agents may be administered in low dosage.

Diagnosis of Neurodegenerative Disorders

The methods and compositions of the present invention may be useful for treating any patient that has been diagnosed with or is at risk of having a neurodegenerative disorder, such as HD. A patient in whom the development of a neurodegenerative disorder is being prevented may or may not have received such a diagnosis. One skilled in the art will understand that a patient may have been subjected to standard tests or may have been identified, without examination, as one at high risk due to the presence of one or more risk factors.

Diagnosis of neurodegenerative disorders, e.g., HD, may be performed using any standard method known in the art, such as those described herein. Methods for diagnosing such disorders are described, for example, in U.S. Pat. Nos. 6,355,481 and 6,210,970, hereby incorporated by reference. HD may be diagnosed and monitored, for example, by performing genetic tests (e.g., by sequencing the htt gene and testing for the presence of an expanded CAG repeat region); neurological examination, e.g., testing body movement, reflexes, eye movement, hearing, or balance, and/or performing brain imaging; evaluating family history of disease; or conducting a psychological or psychiatric interview. Symptoms or altered behavior that can lead to a diagnosis of HD include, e.g., aggression, altered sexuality, anxiety, apathy, delusions, denial, depression, disinhibition, frustration, hallucinations, irritability, mania, repetition, and lack of awareness.

A patient may be diagnosed as having or being at risk of having HD if a genetic test is performed and the number of CAG repeats in the htt gene is greater than a threshold number, e.g., 38. A larger number of repeats is generally associated with an earlier onset of disease. Similar diagnostic methods may be used, e.g., for any of the polyglutamine expansion disorders. In addition, genetic, neurological, and/or behavioral testing may be used to diagnose other neurodegenerative disorders.

Therapeutic Agents

The present invention involves the administration of an effective amount of one, two, three, four, or more agents selected independently from any of the agents of Tables 1a and 1b to a patient having or being at risk of having, a neurodegenerative disorder, thereby treating, preventing, or ameliorating such a disorder.

In the case of HD, for example, an agent of the invention may inhibit the disease-causing activity of a mutant Htt protein, e.g., by preventing or reducing Htt aggregation.

Analogs of any of the compounds listed in Tables 1a and 1b may be used in any of the methods, kits, and compositions of the invention. Analogs are described herein.

Acetohexamide

Analogs of acetohexamide, an anti-diabetic agent, are chlorpropamide, tolazamide, tolbutamide, phenformin, sulfonylurea glyburide, glypizide, glycazide, glisoxepid, glibomuride, tolbutamide, gliclozide, gliquidone, glyhexamide, phenbentamide, tolcyclamide, 5-(4-[2-[1-(4-2′-pyridylphenyl)ethylideneaminooxy]ethoxy]benzyl]thiazolidine-2,4-dione, 5-(4-[5-methoxy-3-methylimidazo[5,4-b]pyridin-2-yl-methoxy)benzyl]thiazolidine-2,4-dione, or its hydrochloride, 5-[4-(6-methoxy-1-methylbenzimidazol-2-yl-methoxy)benzyl]thiazolidine-2,4-dione, 5-[4-(1-methylbenzimidazol-2-ylmethoxy)-benzyl]thiazolidine-2,4-dione, 5-[4-(5-hydroxy-1,4,6,7-tetramethylbenzimidazol-2-ylmethoxy)benzyl]thiazolidine-2,4-dione, 3-chloro-3-phenyl-1-(3H)-isobenzofuranone, 3-chloro-3-(para-fluorophenyl)-1-(3H)-isobenzofuranone, 3-chloro-3-phenyl-5-bromo-1-(3H)-isobenzofuranone, 3-chloro-3-phenyl-5,6-dimethoxy-1-(3H)-isobenzofuranone, 3-chloro-3-(2-thienyl)-2-(3H)-isobenzofuranone, 3-chloro-3-phenyl-5-methoxy-1-(3H)-isobenzofuranone, 3-chloro-3-phenyl-5,6-methylenedioxy-1-(3H)-isobenzofuranone, 3-chloro-3-(meta-trifluoromethylphenyl)-1-(3H)-isobenzofuranone, 3-chloro-3-(para-chlorophenyl)-1-(3H)-isobenzofuranone, 3-chloro-3-(meta-fluorophenyl)-1-(3H)-isobenzofuranone, and 3-chloro-3-(ortho-fluorophenyl)-1-(3H)-isobenzofuranone. Acetohexamide analogs are described in British patent GB 912,789.

Alsterpaullone

Alsterpaullone (9-nitropaullone), a GSK-3β inhibitor and CDK inhibitor, is described in U.S. Pat. No. 7,232,814. Alsterpaullone analogs are represented by formula (I): 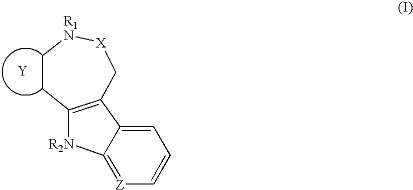
wherein X represents a C═O, C—S—CH₃, C—S, or CNHOH; Z represents C or N; Y represents, with the adjacent ring, a phenyl or thiazolyl residue; the ring or rings being optionally substituted by one or more halogen atoms, hydroxy, alkylenehydroxy, alkynealkylenehydroxy, alkynehydroxycyclohexyl, alkyl, alkoxy, alkylenealkoxy, alkylenecyano groups, the alkylene group being saturated or unsaturated, the radicals being straight-chain or branched and having 1 to 18 carbon atoms, the chain being optionally substituted by one or more hydroxy or amino groups; one or more trifluoromethyl; —COM; —COOM; or —CH₂COOM groups (with M representing a hydrogen atom, a C₁ to C₁₈ alkyl group, straight-chain or branched, and optionally substituted by one or more hydroxy and/or amino; nitroso; nitro; or cyano groups; R₁ represents a hydrogen atom or a C₁ to C₅ alkyl group, R₂ represents a hydrogen atom, or a —C—CO₂—(CH₃)₃ group.

Exemplary asterpaullone analogs are 9-cyano-2,3-dimethoxypaullone; 2-iodopaullone; 2-bromo-9-nitropaullone; 2,3-dimethoxy-9-nitropaullone; 7-bromo-5-(4-nitrophenylhydrazono)-4,5-dihydro-1-H-[1]benzazepin-2(3H)-one; 7,8-dimethoxy-5-(4-nitrophenylhydrazono)-4,5-dihydro-1H-[1]benzazepin-2-(3H)-one; 9-cyanopaullone; kenpaullone (9-bromopaullone); 9-chloropaullone; 9-trifluoromethylpaullone; 2,3-dimethoxy-9-trifluoromethylpaullone; 9-bromo-12-methyloxycarbonylmethylpaullone; 9-fluoropaullone; 9-bromo-2,3-dimethoxypaullone; 9-bromo-2,3-dimethoxypaullone; 9-methylpaullone; 10-bromopaullone; 2-bromopaullone; 11-chloropaullone; 2-(3-hydroxy-1-propinyl)-9-trifluoromethylpaullone; 9-bromo-12-(2-hydroxyethyl)-paullone; 9-bromo-12-methylpaullone; 9-bromo-5-(methyloxycarbonylmethyl)paullone; 11-methylpaullone; paullone; 11-ethylpaullone; 9-bromo-7,12-dihydro-6-(hydroxyamino)-indolo[2-3-d][1]benzazepine; 2,9-dibromopaullone; 11-bromopaullone; 2,3-dimethoxypaullone; 9-bromo-7,12-dihydro-6-methylthio-indolo[2-3-d][1]benzazepine; (E)-2-(3-oxo-1-butenyl)-9-trifluoromethylpaullone; 9-bromo-12-ethylpaullone; 9-bromo-7,12-dihydro-indolo[2-3-d][1]benzazepine-6(5H)-thione; 2-bromo-9-trifluoromethylpaullone; 2-[2-(1-hydroxycyclohexyl)-ethinyl]-9-trifluoromethyl-paullone; 9-bromo-5-methylpaullone; 9-methoxypaullone; 2-iodo-9-trifluoromethylpaullone; 9-bromo-12-(tert-butyloxycarbonyl)-paullone; 9-bromo-12-(2-propenyl)-paullone; 9-bromo-4-hydroxypaullone; 8,10-dichloropaullone; 5-benzyl-9-bromopaullone; 9-bromo-4-methoxypaullone; 9-bromo-5-ethylpaullone; 9-bromo-5,7-bis-(tert-butyloxycarbonyl)-paullone; 4-methoxypaullone; 9-bromo-5,6,7,12-tetrahydrobenzo[6-7]cyclohept[1,2.b]indole; 2-phenyl-4-(2-thienyl)-5H-pyrido[2-3-d][1]benzazepine-6(7H)-thione; 9-bromo-5,7,12-tri-(tert-butyloxycarbonyl)-paullone; 9-bromo-5,12-bis-(tert-butyloxycarbonyl)-paullone; 4-(4-chlorophenyl)-2-(2-naphthyl)-5H-pyrido[2-3-d][1]benzazepine-6(7H)-thione; and 5,6,7,12-tetrahydro-benzo[6-7]cyclohept[1,2-b]indole.

BML-248

Analogs of BML-248 (also referred to as Mu-Phe-hPhe-FMK, N-morpholineurea-phenylalanyl-homophenylalanylfluoromethyl ketone, or Mu-F-hF-FMK), a protease inhibitor, are calpain inhibitor-2 (Mu-F-hF-FMK), Z-Phe-AlaCH₂F, FMK024 (an inhibitor of the major lysosomal protease), trypanopain, Cbz-L-Leu-L-Abu-CONH-Et, Cbz-L-Leu-L-Norvaline-CONH-Et, Cbz-L-Leu-L-Phenylalanine-CONH-Et, Cbz-L-Leu-D-Phe-CONH-Et, Cbz-L-Leu-L-Phe-C(O)-Phe-Ome, Cbz-L-Leu-L-Phe-C(O)-Tyr(O-t-butyl)-Ome, Cbz-L-Leu-L-Phe-C(O)-L-Norleucine-Ome, Cbz-L-Leu-L-Phe-C(O)-L-Ala-OH, Morpholinourea-L-Leu-L-Abu-CONH-Et, dimethylurea-L-Leu-L-Abu-CONH-Et, Boc-L-Leu-L-Abu-CONH-Et, Boc-D-Phe-L-Leu-L-Norvaline-CONH-Et, ritonavir, saquinavir, indinavir, nelfinavir, amprenavir, N-Cbz-L-Leu-L-Leu-L-Leuene-phenyl vinyl sulfone, N-Cbz-L-Leu-L-Leu-D-Leuene-phenyl vinyl sulfone, N-(THIQ-carbonyl)-L-Leu-L-Leu-D-Leuene phenyl vinyl sulfone, N-(THIQ-carbonyl)-L-Leu-L-Leu-L-Leuene phenyl vinyl sulfone, N-(THIQ-carbonyl)-L-Val-L-Met-L-Leuene phenyl vinyl sulfone, N-(THIQ-carbonyl)-L-Val-L-Met-D-Leuene phenyl vinyl sulfone, N-(THIQ-carbonyl)-L-Val-L-Leu-L-Leuene phenyl vinyl sulfone, N-(THIQ-carbonyl)-L-Val-L-Leu-D-Leuene phenyl vinyl sulfone, N-(4-benzylpiperidinyl-carbonyl)-L-Leu-L-Leu-L-Leuene phenyl vinyl sulfone, N-(4-benzylpiperidinyl-carbonyl)-L-Leu-L-Leu-D-Leuene phenyl vinyl sulfone, N-(4-benzylpiperazinyl-carbonyl)-L-Leu-L-Leu-L-Leuene phenyl vinyl sulfone, and N-(4-benzylpiperazinyl-carbonyl)-L-Leu-L-Leu-D-Leuene phenyl vinyl sulfone. BML-248 analogs are described in Couto et al., J. Neurosci. Res. 77:410-419, 2004; Esser et al., Arthritis Rheum. 37:236-247, 1994; Van Noorden et al., Biochem. Biophys. Res. Commun. 178:178-184, 1991; Van Noorden et al., Clin. Exp. Metastasis 16:159-167, 1998; Triggs et al., Eukaryot. Cell, 2:76-83, 2003; and Wasilewski et al., Mol. Biochem. Parasitol. 81:179-189, 1996.

Calcitriol

Analogs of calcitriol, a vitamin D3 receptor, are BXL-353, 22-oxacalcitriol, calcipotriol (MC 903), EB 1089, (5Z,7E,22E)-(1S,3R,24R)-25-(5-propyloxazol-2-yl)-26,27-cyclo-9,10-secocholesta-5,7,10(19),22-tetraene-1,3,24-triol, (5Z,7E,22E)-(1S,3R,24S)-25-(5-propyloxazol-2-yl)-26,27-cyclo-9,10-secocholesta-5,7,10(19),22-tetraene-1,3,24-triol, (5Z,7E,22E)-(1S,3R,24R)-25-(5-methyloxazol-2-yl)-26,27-cyclo-9,10-secocholesta-5,7,10(19),22-tetraene-1,3,24-triol, (5Z,7E,22E)-(1S,3R,24S)-25-(5-methyloxazol-2-yl)-26,27-cyclo-9,10-secocholesta-5,7,10(19),22-tetraene-1,3,24-triol, (5Z,7E,22E)-(1S,3R,24R)-25-(5-ethyloxazol-2-yl)-26,27-cyclo-9,10-secocholesta-5,7,10(19),22-tetraene-1,3,24-triol, (5Z,7E,22E)-(1S,3R,24S)-25-(5-ethyloxazol-2-yl)-26,27-cyclo-9,10-secocholesta-5,7,10(19),22-tetraene-1,3,24-triol, (5Z,7E,22E)-(1S,3R,24R)-25-(5-butyloxazol-2-yl)-26,27-cyclo-9,10-secocholesta-5,7,10(19),22-tetraene-1,3,24-triol, (5Z,7E,22E)-(1S,3R,24S)-25-(5-butyloxazol-2-yl)-26,27-cyclo-9,10-secocholesta-5,7,10(19),22-tetraene-1,3,24-triol, (5Z,7E,22E)-(1S,3R,24R)-25-(5-pentyloxazol-2-yl)-26,27-cyclo-9,10-secocholesta-5,7,10(19),22-tetraene-1,3,24-triol, (5Z,7E,22E)-(1S,3R,24S)-25-(5-pentyloxazol-2-yl)-26,27-cyclo-9,10-secocholesta-5,7,10(19),22-tetraene-1,3,24-triol, (5Z,7E,22E)-(1S,3R,24R)-25-(5-propylthiazol-2-yl)-26,27-cyclo-9,10-secocholesta-5,7,10(19),22-tetraene-1,3,24-triol, (5Z,7E,22E)-(1S,3R,24S)-25-(5-propylthiazol-2-yl)-26,27-cyclo-9,10-secocholesta-5,7,10(19),22-tetraene-1,3,24-triol, (5Z,7E,22E)-(1S,3R,24R)-25-(5-methylthiazol-2-yl)-26,27-cyclo-9,10-secocholesta-5,7,10(19),22-tetraene-1,3,24-triol, (5Z,7E,22E)-(1S,3R,24S)-25-(5-methylthiazol-2-yl)-26,27-cyclo-9,10-secocholesta-5,7,10(19),22-tetraene-1,3,24-triol, (5Z,7E,22E)-(1S,3R,24R)-25-(5-ethylthiazol-2-yl)-26,27-cyclo-9,10-secocholesta-5,7,10(19),22-tetraene-1,3,24-triol, (5Z,7E,22E)-(1S,3R,24S)-25-(5-ethylthiazol-2-yl)-26,27-cyclo-9,10-secocholesta-5,7,10(19),22-tetraene-1,3,24-triol, (5Z,7E,22E)-(1S,3R,24R)-25-(5-butylthiazol-2-yl)-26,27-cyclo-9,10-secocholesta-5,7,10(19),22-tetraene-1,3,24-triol, (5Z,7E,22E)-(1S,3R,24S)-25-(5-butylthiazol-2-yl)-26,27-cyclo-9,10-secocholesta-5,7,10(19),22-tetraene-1,3,24-triol, (5Z,7E,22E)-(1S,3R,24R)-25-(5-pentylthiazol-2-yl)-26,27-cyclo-9,10-secocholesta-5,7,10(19),22-tetraene-1,3,24-triol, (5Z,7E,22E)-(1S,3R,24S)-25-(5-pentylthiazol-2-yl)-26,27-cyclo-9,10-secocholesta-5,7,10(19),22-tetraene-1,3,24-triol, (5Z,7E,22E)-(1S,3R,24R)-25-(5-propylimidazol-2-yl)-26,27-cyclo-9,10-secocholesta-5,7,10(19),22-tetraene-1,3,24-triol, (5Z,7E,22E)-(1S,3R,24S)-25-(5-propylimidazol-2-yl)-26,27-cyclo-9,10-secocholesta-5,7,10(19),22-tetraene-1,3,24-triol, (5Z,7E,22E)-(1S,3R,24R)-25-(5-methylimidazol-2-yl)-26,27-cyclo-9,10-secocholesta-5,7,10(19),22-tetraene-1,3,24-triol, (5Z,7E,22E)-(1S,3R,24S)-25-(5-methylimidazol-2-yl)-26,27-cyclo-9,10-secocholesta-5,7,10(19),22-tetraene-1,3,24-triol, (5Z,7E,22E)-(1S,3R,24R)-25-(5-ethylimidazol-2-yl)-26,27-cyclo-9,10-secocholesta-5,7,10(19),22-tetraene-1,3,24-triol, (5Z,7E,22E)-(1S,3R,24S)-25-(5-ethylimidazol-2-yl)-26,27-cyclo-9,10-secocholesta-5,7,10(19),22-tetraene-1,3,24-triol, (5Z,7E,22E)-(1S,3R,24R)-25-(5-butylimidazol-2-yl)-26,27-cyclo-9,10-secocholesta-5,7,10(19),22-tetraene-1,3,24-triol, (5Z,7E,22E)-(1S,3R,24S)-25-(5-butylimidazol-2-yl)-26,27-cyclo-9,10-secocholesta-5,7,10(19),22-tetraene-1,3,24-triol, (5Z,7E,22E)-(1S,3R,24R)-25-(5-pentylimidazol-2-yl)-26,27-cyclo-9,10-secocholesta-5,7,10(19),22-tetraene-1,3,24-triol, (5Z,7E,22E)-(1S,3R,24S)-25-(5-pentylimidazol-2-yl)-26,27-cyclo-9,10-secocholesta-5,7,10(19),22-tetraene-1,3,24-triol, (5Z,7E,22E)-(1S,3R,24R)-25-(5-propylfuran-2-yl)-26,27-cyclo-9,10-secocholesta-5,7,10(19),22-tetraene-1,3,24-triol, (5Z,7E,22E)-(1S,3R,24S)-25-(5-propylfuran-2-yl)-26,27-cyclo-9,10-secocholesta-5,7,10(19),22-tetraene-1,3,24-triol, (5Z,7E,22E)-(1S,3R,24R)-25-(5-methylfuran-2-yl)-26,27-cyclo-9,10-secocholesta-5,7,10(19),22-tetraene-1,3,24-triol, (5Z,7E,22E)-(1S,3R,24S)-25-(5-methylfuran-2-yl)-26,27-cyclo-9,10-secocholesta-5,7,10(19),22-tetraene-1,3,24-triol, (5Z,7E,22E)-(1S,3R,24R)-25-(5-ethylfuran-2-yl)-26,27-cyclo-9,10-secocholesta-5,7,10(19),22-tetraene-1,3,24-triol, (5Z,7E,22E)-(1S,3R,24S)-25-(5-ethylfuran-2-yl)-26,27-cyclo-9,10-secocholesta-5,7,10(19),22-tetraene-1,3,24-triol, (5Z,7E,22E)-(1S,3R,24R)-25-(5-butylfuran-2-yl)-26,27-cyclo-9,10-secocholesta-5,7,10(19),22-tetraene-1,3,24-triol, (5Z,7E,22E)-(1S,3R,24S)-25-(5-butylfuran-2-yl)-26,27-cyclo-9,10-secocholesta-5,7,10(19),22-tetraene-1,3,24-triol, (5Z,7E,22E)-(1S,3R,24R)-25-(5-pentylfuran-2-yl)-26,27-cyclo-9,10-secocholesta-5,7,10(19),22-tetraene-1,3,24-triol, (5Z,7E,22E)-(1S,3R,24S)-25-(5-pentylfuran-2-yl)-26,27-cyclo-9,10-secocholesta-5,7,10(19),22-tetraene-1,3,24-triol, (5Z,7E,22E)-(1S,3R,24R)-25-(5-propylthiophen-2-yl)-26,27-cyclo-9,10-secocholesta-5,7,10(19),22-tetraene-1,3,24-triol, (5Z,7E,22E)-(1S,3R,24S)-25-(5-propylthiophen-2-yl)-26,27-cyclo-9,10-secocholesta-5,7,10(19),22-tetraene-1,3,24-triol, (5Z,7E,22E)-(1S,3R,24R)-25-(5-methylthiophen-2-yl)-26,27-cyclo-9,10-secocholesta-5,7,10(19),22-tetraene-1,3,24-triol, (5Z,7E,22E)-(1S,3R,24S)-25-(5-methylthiophen-2-yl)-26,27-cyclo-9,10-secocholesta-5,7,10(19),22-tetraene-1,3,24-triol, (5Z,7E,22E)-(1S,3R,24R)-25-(5-ethylthiophen-2-yl)-26,27-cyclo-9,10-secocholesta-5,7,10(19),22-tetraene-1,3,24-triol, (5Z,7E,22E)-(1S,3R,24S)-25-(5-ethylthiophen-2-yl)-26,27-cyclo-9,10-secocholesta-5,7,10(19),22-tetraene-1,3,24-triol, (5Z,7E,22E)-(1S,3R,24R)-25-(5-butylthiophen-2-yl)-26,27-cyclo-9,10-secocholesta-5,7,10(19),22-tetraene-1,3,24-triol, (5Z,7E,22E)-(1S,3R,24S)-25-(5-butylthiophen-2-yl)-26,27-cyclo-9,10-secocholesta-5,7,10(19),22-tetraene-1,3,24-triol, (5Z,7E,22E)-(1S,3R,24R)-25-(5-pentylthiophen-2-yl)-26,27-cyclo-9,10-secocholesta-5,7,10(19),22-tetraene-1,3,24-triol, (5Z,7E,22E)-(1S,3R,24S)-25-(5-pentylthiophen-2-yl)-26,27-cyclo-9,10-secocholesta-5,7,10(19),22-tetraene-1,3,24-triol, (5Z,7E,22E)-(1S,3R,24R)-25-(5-propylpyrrol-2-yl)-26,27-cyclo-9,10-secocholesta-5,7,10(19),22-tetraene-1,3,24-triol, (5Z,7E,22E)-(1S,3R,24S)-25-(5-propylpyrrol-2-yl)-26,27-cyclo-9,10-secocholesta-5,7,10(19),22-tetraene-1,3,24-triol, (5Z,7E,22E)-(1S,3R,24R)-25-(5-methylpyrrol-2-yl)-26,27-cyclo-9,10-secocholesta-5,7,10(19),22-tetraene-1,3,24-triol, (5Z,7E,22E)-(1S,3R,24S)-25-(5-methylpyrrol-2-yl)-26,27-cyclo-9,10-secocholesta-5,7,10(19),22-tetraene-1,3,24-triol, (5Z,7E,22E)-(1S,3R,24R)-25-(5-ethylpyrrol-2-yl)-26,27-cyclo-9,10-secocholesta-5,7,10(19),22-tetraene-1,3,24-triol, (5Z,7E,22E)-(1S,3R,24S)-25-(5-ethylpyrrol-2-yl)-26,27-cyclo-9,10-secocholesta-5,7,10(19),22-tetraene-1,3,24-triol, (5Z,7E,22E)-(1S,3R,24R)-25-(5-butylpyrrol-2-yl)-26,27-cyclo-9,10-secocholesta-5,7,10(19),22-tetraene-1,3,24-triol, (5Z,7E,22E)-(1S,3R,24S)-25-(5-butylpyrrol-2-yl)-26,27-cyclo-9,10-secocholesta-5,7,10(19),22-tetraene-1,3,24-triol, (5Z,7E,22E)-(1S,3R,24R)-25-(5-pentylpyrrol-2-yl)-26,27-cyclo-9,10-secocholesta-5,7,10(19),22-tetraene-1,3,24-triol, (5Z,7E,22E)-(1S,3R,24S)-25-(5-pentylpyrrol-2-yl)-26,27-cyclo-9,10-secocholesta-5,7,10(19),22-tetraene-1,3,24-triol, (5Z,7E,22E)-(1S,3R,24R)-25-(4-propyloxazol-2-yl)-26,27-cyclo-9,10-secocholesta-5,7,10(19),22-tetraene-1,3,24-triol, (5Z,7E,22E)-(1S,3R,24S)-25-(4-propyloxazol-2-yl)-26,27-cyclo-9,10-secocholesta-5,7,10(19),22-tetraene-1,3,24-triol, (5Z,7E,22E)-(1S,3R,24R)-25-(4-methyloxazol-2-yl)-26,27-cyclo-9,10-secocholesta-5,7,10(19),22-tetraene-1,3,24-triol, (5Z,7E,22E)-(1S,3R,24S)-25-(4-methyloxazol-2-yl)-26,27-cyclo-9,10-secocholesta-5,7,10(19),22-tetraene-1,3,24-triol, (5Z,7E,22E)-(1S,3R,24R)-25-(4-ethyloxazol-2-yl)-26,27-cyclo-9,10-secocholesta-5,7,10(19),22-tetraene-1,3,24-triol, (5Z,7E,22E)-(1S,3R,24S)-25-(4-ethyloxazol-2-yl)-26,27-cyclo-9,10-secocholesta-5,7,10(19),22-tetraene-1,3,24-triol, (5Z,7E,22E)-(1S,3R,24R)-25-(4-butyloxazol-2-yl)-26,27-cyclo-9,10-secocholesta-5,7,10(19),22-tetraene-1,3,24-triol, (5Z,7E,22E)-(1S,3R,24S)-25-(4-butyloxazol-2-yl)-26,27-cyclo-9,10-secocholesta-5,7,10(19),22-tetraene-1,3,24-triol, (5Z,7E,22E)-(1S,3R,24R)-25-(4-pentyloxazol-2-yl)-26,27-cyclo-9,10-secocholesta-5,7,10(19),22-tetraene-1,3,24-triol, (5Z,7E,22E)-(1S,3R,24S)-25-(4-pentyloxazol-2-yl)-26,27-cyclo-9,10-secocholesta-5,7,10(19),22-tetraene-1,3,24-triol, (5Z,7E,22E)-(1S,3R,24R)-25-(4-propylthiazol-2-yl)-26,27-cyclo-9,10-secocholesta-5,7,10(19),22-tetraene-1,3,24-triol, (5Z,7E,22E)-(1S,3R,24S)-25-(4-propylthiazol-2-yl)-26,27-cyclo-9,10-secocholesta-5,7,10(19),22-tetraene-1,3,24-triol, (5Z,7E,22E)-(1S,3R,24R)-25-(4-methylthiazol-2-yl)-26,27-cyclo-9,10-secocholesta-5,7,10(19),22-tetraene-1,3,24-triol, (5Z,7E,22E)-(1S,3R,24S)-25-(4-methylthiazol-2-yl)-26,27-cyclo-9,10-secocholesta-5,7,10(19),22-tetraene-1,3,24-triol, (5Z,7E,22E)-(1S,3R,24R)-25-(4-ethylthiazol-2-yl)-26,27-cyclo-9,10-secocholesta-5,7,10(19),22-tetraene-1,3,24-triol, (5Z,7E,22E)-(1S,3R,24S)-25-(4-ethylthiazol-2-yl)-26,27-cyclo-9,10-secocholesta-5,7,10(19),22-tetraene-1,3,24-triol, (5Z,7E,22E)-(1S,3R,24R)-25-(4-butylthiazol-2-yl)-26,27-cyclo-9,10-secocholesta-5,7,10(19),22-tetraene-1,3,24-triol, (5Z,7E,22E)-(1S,3R,24S)-25-(4-butylthiazol-2-yl)-26,27-cyclo-9,10-secocholesta-5,7,10(19),22-tetraene-1,3,24-triol, (5Z,7E,22E)-(1S,3R,24R)-25-(4-pentylthiazol-2-yl)-26,27-cyclo-9,10-secocholesta-5,7,10(19),22-tetraene-1,3,24-triol, (5Z,7E,22E)-(1S,3R,24S)-25-(4-pentylthiazol-2-yl)-26,27-cyclo-9,10-secocholesta-5,7,10(19),22-tetraene-1,3,24-triol, (5Z,7E,22E)-(1S,3R,24R)-25-(4-propylimidazol-2-yl)-26,27-cyclo-9,10-secocholesta-5,7,10(19),22-tetraene-1,3,24-triol, (5Z,7E,22E)-(1S,3R,24S)-25-(4-propylimidazol-2-yl)-26,27-cyclo-9,10-secocholesta-5,7,10(19),22-tetraene-1,3,24-triol, (5Z,7E,22E)-(1S,3R,24R)-25-(4-methylimidazol-2-yl)-26,27-cyclo-9,10-secocholesta-5,7,10(19),22-tetraene-1,3,24-triol, (5Z,7E,22E)-(1S,3R,24S)-25-(4-methylimidazol-2-yl)-26,27-cyclo-9,10-secocholesta-5,7,10(19),22-tetraene-1,3,24-triol, (5Z,7E,22E)-(1S,3R,24R)-25-(4-ethylimidazol-2-yl)-26,27-cyclo-9,10-secocholesta-5,7,10(19),22-tetraene-1,3,24-triol, (5Z,7E,22E)-(1S,3R,24S)-25-(4-ethylimidazol-2-yl)-26,27-cyclo-9,10-secocholesta-5,7,10(19),22-tetraene-1,3,24-triol, (5Z,7E,22E)-(1S,3R,24R)-25-(4-butylimidazol-2-yl)-26,27-cyclo-9,10-secocholesta-5,7,10(19),22-tetraene-1,3,24-triol, (5Z,7E,22E)-(1S,3R,24S)-25-(4-butylimidazol-2-yl)-26,27-cyclo-9,10-secocholesta-5,7,10(19),22-tetraene-1,3,24-triol, (5Z,7E,22E)-(1S,3R,24R)-25-(4-pentylimidazol-2-yl)-26,27-cyclo-9,10-secocholesta-5,7,10(19),22-tetraene-1,3,24-triol, (5Z,7E,22E)-(1S,3R,24S)-25-(4-pentylimidazol-2-yl)-26,27-cyclo-9,10-secocholesta-5,7,10(19),22-tetraene-1,3,24-triol, (5Z,7E,22E)-(1S,3R,24R)-25-(4-propylfuran-2-yl)-26,27-cyclo-9,10-secocholesta-5,7,10(19),22-tetraene-1,3,24-triol, (5Z,7E,22E)-(1S,3R,24S)-25-(4-propylfuran-2-yl)-26,27-cyclo-9,10-secocholesta-5,7,10(19),22-tetraene-1,3,24-triol, (5Z,7E,22E)-(1S,3R,24R)-25-(4-methylfuran-2-yl)-26,27-cyclo-9,10-secocholesta-5,7,10(19),22-tetraene-1,3,24-triol, (5Z,7E,22E)-(1S,3R,24S)-25-(4-methylfuran-2-yl)-26,27-cyclo-9,10-secocholesta-5,7,10(19),22-tetraene-1,3,24-triol, (5Z,7E,22E)-(1S,3R,24R)-25-(4-ethylfuran-2-yl)-26,27-cyclo-9,10-secocholesta-5,7,10(19),22-tetraene-1,3,24-triol, (5Z,7E,22E)-(1S,3R,24S)-25-(4-ethylfuran-2-yl)-26,27-cyclo-9,10-secocholesta-5,7,10(19),22-tetraene-1,3,24-triol, (5Z,7E,22E)-(1S,3R,24R)-25-(4-butylfuran-2-yl)-26,27-cyclo-9,10-secocholesta-5,7,10(19),22-tetraene-1,3,24-triol, (5Z,7E,22E)-(1S,3R,24S)-25-(4-butylfuran-2-yl)-26,27-cyclo-9,10-secocholesta-5,7,10(19),22-tetraene-1,3,24-triol, (5Z,7E,22E)-(1S,3R,24R)-25-(4-pentylfuran-2-yl)-26,27-cyclo-9,10-secocholesta-5,7,10(19),22-tetraene-1,3,24-triol, (5Z,7E,22E)-(1S,3R,24S)-25-(4-pentylfuran-2-yl)-26,27-cyclo-9,10-secocholesta-5,7,10(19),22-tetraene-1,3,24-triol, (5Z,7E,22E)-(1S,3R,24R)-25-(4-propylthiophen-2-yl)-26,27-cyclo-9,10-secocholesta-5,7,10(19),22-tetraene-1,3,24-triol, (5Z,7E,22E)-(1S,3R,24S)-25-(4-propylthiophen-2-yl)-26,27-cyclo-9,10-secocholesta-5,7,10(19),22-tetraene-1,3,24-triol, (5Z,7E,22E)-(1S,3R,24R)-25-(4-methylthiophen-2-yl)-26,27-cyclo-9,10-secocholesta-5,7,10(19),22-tetraene-1,3,24-triol, (5Z,7E,22E)-(1S,3R,24S)-25-(4-methylthiophen-2-yl)-26,27-cyclo-9,10-secocholesta-5,7,10(19),22-tetraene-1,3,24-triol, (5Z,7E,22E)-(1S,3R,24R)-25-(4-ethylthiophen-2-yl)-26,27-cyclo-9,10-secocholesta-5,7,10(19),22-tetraene-1,3,24-triol, (5Z,7E,22E)-(1S,3R,24S)-25-(4-ethylthiophen-2-yl)-26,27-cyclo-9,10-secocholesta-5,7,10(19),22-tetraene-1,3,24-triol, (5Z,7E,22E)-(1S,3R,24R)-25-(4-butylthiophen-2-yl)-26,27-cyclo-9,10-secocholesta-5,7,10(19),22-tetraene-1,3,24-triol, (5Z,7E,22E)-(1S,3R,24S)-25-(4-butylthiophen-2-yl)-26,27-cyclo-9,10-secocholesta-5,7,10(19),22-tetraene-1,3,24-triol, (5Z,7E,22E)-(1S,3R,24R)-25-(4-pentylthiophen-2-yl)-26,27-cyclo-9,10-secocholesta-5,7,10(19),22-tetraene-1,3,24-triol, (5Z,7E,22E)-(1S,3R,24S)-25-(4-pentylthiophen-2-yl)-26,27-cyclo-9,10-secocholesta-5,7,10(19),22-tetraene-1,3,24-triol, (5Z,7E,22E)-(1S,3R,24R)-25-(4-propylpyrrol-2-yl)-26,27-cyclo-9,10-secocholesta-5,7,10(19),22-tetraene-1,3,24-triol, (5Z,7E,22E)-(1S,3R,24S)-25-(4-propylpyrrol-2-yl)-26,27-cyclo-9,10-secocholesta-5,7,10(19),22-tetraene-1,3,24-triol, (5Z,7E,22E)-(1S,3R,24R)-25-(4-methylpyrrol-2-yl)-26,27-cyclo-9,10-secocholesta-5,7,10(19),22-tetraene-1,3,24-triol, (5Z,7E,22E)-(1S,3R,24S)-25-(4-methylpyrrol-2-yl)-26,27-cyclo-9,10-secocholesta-5,7,10(19),22-tetraene-1,3,24-triol, (5Z,7E,22E)-(1S,3R,24R)-25-(4-ethylpyrrol-2-yl)-26,27-cyclo-9,10-secocholesta-5,7,10(19),22-tetraene-1,3,24-triol, (5Z,7E,22E)-(1S,3R,24S)-25-(4-ethylpyrrol-2-yl)-26,27-cyclo-9,10-secocholesta-5,7,10(19),22-tetraene-1,3,24-triol, (5Z,7E,22E)-(1S,3R,24R)-25-(4-butylpyrrol-2-yl)-26,27-cyclo-9,10-secocholesta-5,7,10(19),22-tetraene-1,3,24-triol, (5Z,7E,22E)-(1S,3R,24S)-25-(4-butylpyrrol-2-yl)-26,27-cyclo-9,10-secocholesta-5,7,10(19),22-tetraene-1,3,24-triol, (5Z,7E,22E)-(1S,3R,24R)-25-(4-pentylpyrrol-2-yl)-26,27-cyclo-9,10-secocholesta-5,7,10(19),22-tetraene-1,3,24-triol, (5Z,7E,22E)-(1S,3R,24S)-25-(4-pentylpyrrol-2-yl)-26,27-cyclo-9,10-secocholesta-5,7,10(19),22-tetraene-1,3,24-triol, (5Z,7E,22E)-(1S,3R,24R)-25-(3-methyl-1,2,4-oxadiazol-5-yl)-26,27-cyclo-9,10-secocholesta-5,7,10(19),22-tetraene-1,3,24-triol, (5Z,7E,22E)-(1S,3R,24S)-25-(3-methyl-1,2,4-oxadiazol-5-yl)-26,27-cyclo-9,10-secocholesta-5,7,10(19),22-tetraene-1,3,24-triol, (5Z,7E,22E)-(1S,3R,24R)-25-(3-ethyl-1,2,4-oxadiazol-5-yl)-26,27-cyclo-9,10-secocholesta-5,7,10(19),22-tetraene-1,3,24-triol, (5Z,7E,22E)-(1S,3R,24S)-25-(3-ethyl-1,2,4-oxadiazol-5-yl)-26,27-cyclo-9,10-secocholesta-5,7,10(19),22-tetraene-1,3,24-triol, (5Z,7E,22E)-(1S,3R,24R)-25-(3-propyl-1,2,4-oxadiazol-5-yl)-26,27-cyclo-9,10-secocholesta-5,7,10(19),22-tetraene-1,3,24-triol, (5Z,7E,22E)-(1S,3R,24S)-25-(3-propyl-1,2,4-oxadiazol-5-yl)-26,27-cyclo-9,10-secocholesta-5,7,10(19),22-tetraene-1,3,24-triol, (5Z,7E,22E)-(1S,3R,24R)-25-(3-butyl-1,2,4-oxadiazol-5-yl)-26,27-cyclo-9,10-secocholesta-5,7,10(19),22-tetraene-1,3,24-triol, (5Z,7E,22E)-(1S,3R,24S)-25-(3-butyl-1,2,4-oxadiazol-5-yl)-26,27-cyclo-9,10-secocholesta-5,7,10(19),22-tetraene-1,3,24-triol, (5Z,7E,22E)-(1S,3R,24R)-25-(3-pentyl-1,2,4-oxadiazol-5-yl)-26,27-cyclo-9,10-secocholesta-5,7,10(19),22-tetraene-1,3,24-triol, (5Z,7E,22E)-(1S,3R,24S)-25-(3-pentyl-1,2,4-oxadiazol-5-yl)-26,27-cyclo-9,10-secocholesta-5,7,10(19),22-tetraene-1,3,24-triol, (5Z,7E,22E)-(1S,3R,24R)-25-(5-methyl-1,3,4-oxadiazol-2-yl)-26,27-cyclo-9,10-secocholesta-5,7,10(19),22-tetraene-1,3,24-triol, (5Z,7E,22E)-(1S,3R,24S)-25-(5-methyl-1,3,4-oxadiazol-2-yl)-26,27-cyclo-9,10-secocholesta-5,7,10(19),22-tetraene-1,3,24-triol, (5Z,7E,22E)-(1S,3R,24R)-25-(5-ethyl-1,3,4-oxadiazol-2-yl)-26,27-cyclo-9,10-secocholesta-5,7,10(19),22-tetraene-1,3,24-triol, (5Z,7E,22E)-(1S,3R,24S)-25-(5-ethyl-1,3,4-oxadiazol-2-yl)-26,27-cyclo-9,10-secocholesta-5,7,10(19),22-tetraene-1,3,24-triol, (5Z,7E,22E)-(1S,3R,24R)-25-(5-propyl-1,3,4-oxadiazol-2-yl)-26,27-cyclo-9,10-secocholesta-5,7,10(19),22-tetraene-1,3,24-triol, (5Z,7E,22E)-(1S,3R,24S)-25-(5-propyl-1,3,4-oxadiazol-2-yl)-26,27-cyclo-9,10-secocholesta-5,7,10(19),22-tetraene-1,3,24-triol, (5Z,7E,22E)-(1S,3R,24R)-25-(5-butyl-1,3,4-oxadiazol-2-yl)-26,27-cyclo-9,10-secocholesta-5,7,10(19),22-tetraene-1,3,24-triol, (5Z,7E,22E)-(1S,3R,24S)-25-(5-butyl-1,3,4-oxadiazol-2-yl)-26,27-cyclo-9,10-secocholesta-5,7,10(19),22-tetraene-1,3,24-triol, (5Z,7E,22E)-(1S,3R,24R)-25-(5-pentyl-1,3,4-oxadiazol-2-yl)-26,27-cyclo-9,10-secocholesta-5,7,10(19),22-tetraene-1,3,24-triol, (5Z,7E,22E)-(1S,3R,24S)-25-(5-pentyl-1,3,4-oxadiazol-2-yl)-26,27-cyclo-9,10-secocholesta-5,7,10(19),22-tetraene-1,3,24-triol, (5Z,7E,22E)-(1S,3R,24R)-25-phenyl-26,27-cyclo-9,10-secocholesta-5,7,10(19), 22-tetraene-1,3,24-triol, (5Z,7E,22E)-(1S,3R,24S)-25-phenyl-26,27-cyclo-9,10-secocholesta-5,7,10(19), 22-tetraene-1,3,24-triol, (5Z,7E,22E)-(1S,3R,24R)-25-(4-methylphenyl)-26,27-cyclo-9,10-secocholesta-5,7,10(19),22-tetraene-1,3,24-triol, (5Z,7E,22E)-(1S,3R,24S)-25-(4-methylphenyl)-26,27-cyclo-9,10-secocholesta-5,7,10(19),22-tetraene-1,3,24-triol, (5Z,7E,22E)-(1S,3R,24R)-25-(4-ethylphenyl)-26,27-cyclo-9,10-secocholesta-5,7,10(19),22-tetraene-1,3,24-triol, (5Z,7E,22E)-(1S,3R,24S)-25-(4-ethylphenyl)-26,27-cyclo-9,10-secocholesta-5,7,10(19),22-tetraene-1,3,24-triol, (5Z,7E,22E)-(1S,3R,24R)-25-(4-propylphenyl)-26,27-cyclo-9,10-secocholesta-5,7,10(19),22-tetraene-1,3,24-triol, (5Z,7E,22E)-(1S,3R,24S)-25-(4-propylphenyl)-26,27-cyclo-9,10-secocholesta-5,7,10(19),22-tetraene-1,3,24-triol, (5Z,7E,22E)-(1S,3R,24R)-25-(4-butylphenyl)-26,27-cyclo-9,10-secocholesta-5,7,10(19),22-tetraene-1,3,24-triol, (5Z,7E,22E)-(1S,3R,24S)-25-(4-butylphenyl)-26,27-cyclo-9,10-secocholesta-5,7,10(19),22-tetraene-1,3,24-triol, (5Z,7E,22E)-(1S,3R,24R)-25-(4-pentylphenyl)-26,27-cyclo-9,10-secocholesta-5,7,10(19),22-tetraene-1,3,24-triol, (5Z,7E,22E)-(1S,3R,24S)-25-(4-pentylphenyl)-26,27-cyclo-9,10-secocholesta-5,7,10(19),22-tetraene-1,3,24-triol, (5Z,7E,22E)-(1S,3R,24R)-25-(3-methylphenyl)-26,27-cyclo-9,10-secocholesta-5,7,10(19),22-tetraene-1,3,24-triol, (5Z,7E,22E)-(1S,3R,24S)-25-(3-methylphenyl)-26,27-cyclo-9,10-secocholesta-5,7,10(19),22-tetraene-1,3,24-triol, (5Z,7E,22E)-(1S,3R,24R)-25-(3-ethylphenyl)-26,27-cyclo-9,10-secocholesta-5,7,10(19),22-tetraene-1,3,24-triol, (5Z,7E,22E)-(1S,3R,24S)-25-(3-ethylphenyl)-26,27-cyclo-9,10-secocholesta-5,7,10(19),22-tetraene-1,3,24-triol, (5Z,7E,22E)-(1S,3R,24R)-25-(3-propylphenyl)-26,27-cyclo-9,10-secocholesta-5,7,10(19),22-tetraene-1,3,24-triol, (5Z,7E,22E)-(1S,3R,24S)-25-(3-propylphenyl)-26,27-cyclo-9,10-secocholesta-5,7,10(19),22-tetraene-1,3,24-triol, (5Z,7E,22E)-(1S,3R,24R)-25-(3-butylphenyl)-26,27-cyclo-9,10-secocholesta-5,7,10(19),22-tetraene-1,3,24-triol, (5Z,7E,22E)-(1S,3R,24S)-25-(3-butylphenyl)-26,27-cyclo-9,10-secocholesta-5,7,10(19),22-tetraene-1,3,24-triol, (5Z,7E,22E)-(1S,3R,24R)-25-(3-pentylphenyl)-26,27-cyclo-9,10-secocholesta-5,7,10(19),22-tetraene-1,3,24-triol, (5Z,7E,22E)-(1S,3R,24S)-25-(3-pentylphenyl)-26,27-cyclo-9,10-secocholesta-5,7,10(19),22-tetraene-1,3,24-triol, (5Z,7E,22E)-(1S,3R,24R)-25-[4-(1-methylethyl)phenyl)-26,27-cyclo-9,10-secocholesta-5,7,10(19),22-tetraene-1,3,24-triol, (5Z,7E,22E)-(1S,3R,24S)-25-[4-(1-methylethyl)phenyl)-26,27-cyclo-9,10-secocholesta-5,7,10(19),22-tetraene-1,3,24-triol, (5Z,7E,22E)-(1S,3R,24R)-25-[3-(1-methylethyl)phenyl)-26,27-cyclo-9,10-secocholesta-5,7,10(19),22-tetraene-1,3,24-triol, (5Z,7E,22E)-(1S,3R,24S)-25-[3-(1-methylethyl)phenyl)-26,27-cyclo-9,10-secocholesta-5,7,10(19),22-tetraene-1,3,24-triol, (5Z,7E,22E)-(1S,3R,24R)-25-(2-pyridyl)-26,27-cyclo-9,10-secocholesta-5,7,10 (19),22-tetraene-1,3,24-triol, (5Z,7E,22E)-(1S,3R,24S)-25-(2-pyridyl)-26,27-cyclo-9,10-secocholesta-5,7,10(19),22-tetraene-1,3,24-triol, (5Z,7E,22E)-(1S,3R,24R)-25-(6-methyl-2-pyridyl)-26,27-cyclo-9,10-secocholesta-5,7,10(19),22-tetraene-1,3,24-triol, (5Z,7E,22E)-(1S,3R,24S)-25-(6-methyl-2-pyridyl)-26,27-cyclo-9,10-secocholesta-5,7,10(19),22-tetraene-1,3,24-triol, (5Z,7E,22E)-(1S,3R,24R)-25-(5-methyl-2-pyridyl)-26,27-cyclo-9,10-secocholesta-5,7,10(19),22-tetraene-1,3,24-triol, (5Z,7E,22E)-(1S,3R,24S)-25-(5-methyl-2-pyridyl)-26,27-cyclo-9,10-secocholesta-5,7,10(19),22-tetraene-1,3,24-triol, (5Z,7E,22E)-(1S,3R,24R)-25-(4-methyl-2-pyridyl)-26,27-cyclo-9,10-secocholesta-5,7,10(19),22-tetraene-1,3,24-triol, (5Z,7E,22E)-(1S,3R,24S)-25-(4-methyl-2-pyridyl)-26,27-cyclo-9,10-secocholesta-5,7,10(19),22-tetraene-1,3,24-triol, (5Z,7E,22E)-(1S,3R,24R)-25-(6-ethyl-2-pyridyl)-26,27-cyclo-9,10-secocholesta-5,7,10(19),22-tetraene-1,3,24-triol, (5Z,7E,22E)-(1S,3R,24S)-25-(6-ethyl-2-pyridyl)-26,27-cyclo-9,10-secocholesta-5,7,10(19),22-tetraene-1,3,24-triol, (5Z,7E,22E)-(1S,3R,24R)-25-(5-ethyl-2-pyridyl)-26,27-cyclo-9,10-secocholesta-5,7,10(19),22-tetraene-1,3,24-triol, (5Z,7E,22E)-(1S,3R,24S)-25-(5-ethyl-2-pyridyl)-26,27-cyclo-9,10-secocholesta-5,7,10(19),22-tetraene-1,3,24-triol, (5Z,7E,22E)-(1S,3R,24R)-25-(4-ethyl-2-pyridyl)-26,27-cyclo-9,10-secocholesta-5,7,10(19),22-tetraene-1,3,24-triol, (5Z,7E,22E)-(1S,3R,24S)-25-(4-ethyl-2-pyridyl)-26,27-cyclo-9,10-secocholesta-5,7,10(19),22-tetraene-1,3,24-triol, (5Z,7E,22E)-(1S,3R,24R)-25-(6-propyl-2-pyridyl)-26,27-cyclo-9,10-secocholesta-5,7,10(19),22-tetraene-1,3,24-triol, (5Z,7E,22E)-(1S,3R,24S)-25-(6-propyl-2-pyridyl)-26,27-cyclo-9,10-secocholesta-5,7,10(19),22-tetraene-1,3,24-triol, (5Z,7E,22E)-(1S,3R,24R)-25-(5-propyl-2-pyridyl)-26,27-cyclo-9,10-secocholesta-5,7,10(19),22-tetraene-1,3,24-triol, (5Z,7E,22E)-(1S,3R,24S)-25-(5-propyl-2-pyridyl)-26,27-cyclo-9,10-secocholesta-5,7,10(19),22-tetraene-1,3,24-triol, (5Z,7E,22E)-(1S,3R,24R)-25-(4-propyl-2-pyridyl)-26,27-cyclo-9,10-secocholesta-5,7,10(19),22-tetraene-1,3,24-triol, (5Z,7E,22E)-(1S,3R,24S)-25-(4-propyl-2-pyridyl)-26,27-cyclo-9,10-secocholesta-5,7,10(19),22-tetraene-1,3,24-triol, (5Z,7E,22E)-(1S,3R,24R)-25-(6-butyl-2-pyridyl)-26,27-cyclo-9,10-secocholesta-5,7,10(19),22-tetraene-1,3,24-triol, (5Z,7E,22E)-(1S,3R,24S)-25-(6-butyl-2-pyridyl)-26,27-cyclo-9,10-secocholesta-5,7,10(19),22-tetraene-1,3,24-triol, (5Z,7E,22E)-(1S,3R,24R)-25-(5-butyl-2-pyridyl)-26,27-cyclo-9,10-secocholesta-5,7,10(19),22-tetraene-1,3,24-triol, (5Z,7E,22E)-(1S,3R,24S)-25-(5-butyl-2-pyridyl)-26,27-cyclo-9,10-secocholesta-5,7,10(19),22-tetraene-1,3,24-triol, (5Z,7E,22E)-(1S,3R,24R)-25-(4-butyl-2-pyridyl)-26,27-cyclo-9,10-secocholesta-5,7,10(19),22-tetraene-1,3,24-triol, (5Z,7E,22E)-(1S,3R,24S)-25-(4-butyl-2-pyridyl)-26,27-cyclo-9,10-secocholesta-5,7,10(19),22-tetraene-1,3,24-triol, (5Z,7E,22E)-(1S,3R,24R)-25-(5,5-dimethyl-2-oxazolin-2-yl)-26,27-cyclo-9,10-secocholesta-5,7,10(19),22-tetraene-1,3,24-triol, (5Z,7E,22E)-(1S,3R,24S)-25-(5,5-dimethyl-2-oxazolin-2-yl)-26,27-cyclo-9,10-secocholesta-5,7,10(19),22-tetraene-1,3,24-triol, (5Z,7E,22E)-(1S,3R,24R)-25-(5,5-diethyl-2-oxazolin-2-yl)-26,27-cyclo-9,10-secocholesta-5,7,10(19),22-tetraene-1,3,24-triol, (5Z,7E,22E)-(1S,3R,24S)-25-(5,5-diethyl-2-oxazolin-2-yl)-26,27-cyclo-9,10-secocholesta-5,7,10(19),22-tetraene-1,3,24-triol, (5Z,7E,22E)-[1S,3R,24R,25(R)]-25-(5-methyl-2-oxazolin-2-yl)-26,27-cyclo-9,10-secocholesta-5,7,10(19),22-tetraene-1,3,24-triol, (5Z,7E,22E)-(1S,3R,24S,25(R)]-25-(5-methyl-2-oxazolin-2-yl)-26,27-cyclo-9,10-secocholesta-5,7,10(19),22-tetraene-1,3,24-triol, (5Z,7E,22E)-[1S,3R,24R,25(S)]-25-(5-methyl-2-oxazolin-2-yl)-26,27-cyclo-9,10-secocholesta-5,7,10(19),22-tetraene-1,3,24-triol, (5Z,7E,22E)-[1S,3R,24S,25(S)]-25-(5-methyl-2-oxazolin-2-yl)-26,27-cyclo-9,10-secocholesta-5,7,10(19),22-tetraene-1,3,24-triol, (5Z,7E,22E)-[1S,3R,24R,25(R)]-25-(5-ethyl-2-oxazolin-2-yl)-26,27-cyclo-9,10-secocholesta-5,7,10(19),22-tetraene-1,3,24-triol, (5Z,7E,22E)-[1S,3R,24S,25(R)]-25-(5-ethyl-2-oxazolin-2-yl)-26,27-cyclo-9,10-secocholesta-5,7,10(19),22-tetraene-1,3,24-triol, (5Z,7E,22E)-[1S,3R,24R,25(S)]-25-(5-ethyl-2-oxazolin-2-yl)-26,27-cyclo-9,10-secocholesta-5,7,10(19),22-tetraene-1,3,24-triol, (5Z,7E,22E)-[1S,3R,24S,25(S)]-25-(5-ethyl-2-oxazolin-2-yl)-26,27-cyclo-9,10-secocholesta-5,7,10(19),22-tetraene-1,3,24-triol, (5Z,7E,22E)-[1S,3R,24R,25(R)]-25-(5-propyl-2-oxazolin-2-yl)-26,27-cyclo-9,10-secocholesta-5,7,10(19),22-tetraene-1,3,24-triol, (5Z,7E,22E)-[1S,3R,24S,25(R)]-25-(5-propyl-2-oxazolin-2-yl)-26,27-cyclo-9,10-secocholesta-5,7,10(19),22-tetraene-1,3,24-triol, (5Z,7E,22E)-[1S,3R,24R,25(S)]-25-(5-propyl-2-oxazolin-2-yl)-26,27-cyclo-9,10-secocholesta-5,7,10(19),22-tetraene-1,3,24-triol, (5Z,7E,22E)-[1S,3R,24S,25(S)]-25-(5-propyl-2-oxazolin-2-yl)-26,27-cyclo-9,10-secocholesta-5,7,10(19),22-tetraene-1,3,24-triol, (5Z,7E,22E)-[1S,3R,24R,25(R)]-25-(5-butyl-2-oxazolin-2-yl)-26,27-cyclo-9,10-secocholesta-5,7,10(19),22-tetraene-1,3,24-triol, (5Z,7E,22E)-[1S,3R,24S,25(R)]-25-(5-butyl-2-oxazolin-2-yl)-26,27-cyclo-9,10-secocholesta-5,7,10(19),22-tetraene-1,3,24-triol, (5Z,7E,22E)-[1S,3R,24R,25(S)]-25-(5-butyl-2-oxazolin-2-yl)-26,27-cyclo-9,10-secocholesta-5,7,10(19),22-tetraene-1,3,24-triol, (5Z,7E,22E)-[1S,3R,24S,25(S)]-25-(5-butyl-2-oxazolin-2-yl)-26,27-cyclo-9,10-secocholesta-5,7,10(19),22-tetraene-1,3,24-triol, (5Z,7E,22E)-[1S,3R,24R,25(R)]-25-(5-phenyl-2-oxazolin-2-yl)-26,27-cyclo-9,10-secocholesta-5,7,10(19),22-tetraene-1,3,24-triol, (5Z,7E,22E)-[1S,3R,24S,25(R)]-25-(5-phenyl-2-oxazolin-2-yl)-26,27-cyclo-9,10-secocholesta-5,7,10(19),22-tetraene-1,3,24-triol, (5Z,7E,22E)-[1S,3R,24R,25(S)]-25-(5-phenyl-2-oxazolin-2-yl)-26,27-cyclo-9,10-secocholesta-5,7,10(19),22-tetraene-1,3,24-triol, (5Z,7E,22E)-[1S,3R,24S,25(S)]-25-(5-phenyl-2-oxazolin-2-yl)-26,27-cyclo-9,10-secocholesta-5,7,10(19),22-tetraene-1,3,24-triol, (5Z,7E,22E)-(1S,3R,20S,24R)-25-(5-propyloxazol-2-yl)-26,27-cyclo-9,10-secocholesta-5,7,10(19),22-tetraene-1,3,24-triol, (5Z,7E,22E)-(1S,3R,20S,24S)-25-(5-propyloxazol-2-yl)-26,27-cyclo-9,10-secocholesta-5,7,10(19),22-tetraene-1,3,24-triol, (5Z,7E,22E)-(1S,3R,20S,24R)-25-(5-methyloxazol-2-yl)-26,27-cyclo-9,10-secocholesta-5,7,10(19),22-tetraene-1,3,24-triol, (5Z,7E,22E)-(1S,3R,20S,24S)-25-(5-methyloxazol-2-yl)-26,27-cyclo-9,10-secocholesta-5,7,10(19),22-tetraene-1,3,24-triol, (5Z,7E,22E)-(1S,3R,20S,24R)-25-(5-ethyloxazol-2-yl)-26,27-cyclo-9,10-secocholesta-5,7,10(19),22-tetraene-1,3,24-triol, (5Z,7E,22E)-(1S,3R,20S,24S)-25-(5-ethyloxazol-2-yl)-26,27-cyclo-9,10-secocholesta-5,7,10(19),22-tetraene-1,3,24-triol, (5Z,7E,22E)-(1S,3R,20S,24R)-25-(5-butyloxazol-2-yl)-26,27-cyclo-9,10-secocholesta-5,7,10(19),22-tetraene-1,3,24-triol, (5Z,7E,22E)-(1S,3R,20S,24S)-25-(5-butyloxazol-2-yl)-26,27-cyclo-9,10-secocholesta-5,7,10(19),22-tetraene-1,3,24-triol, (5Z,7E,22E)-(1S,3R,20S,24R)-25-(5-pentyloxazol-2-yl)-26,27-cyclo-9,10-secocholesta-5,7,10(19),22-tetraene-1,3,24-triol, (5Z,7E,22E)-(1S,3R,20S,24S)-25-(5-pentyloxazol-2-yl)-26,27-cyclo-9,10-secocholesta-5,7,10(19),22-tetraene-1,3,24-triol, (5Z,7E,22E)-(1S,3R,20S,24R)-25-(5-propylthiazol-2-yl)-26,27-cyclo-9,10-secocholesta-5,7,10(19),22-tetraene-1,3,24-triol, (5Z,7E,22E)-(1S,3R,20S,24S)-25-(5-propylthiazol-2-yl)-26,27-cyclo-9,10-secocholesta-5,7,10(19),22-tetraene-1,3,24-triol, (5Z,7E,22E)-(1S,3R,20S,24R)-25-(5-methylthiazol-2-yl)-26,27-cyclo-9,10-secocholesta-5,7,10(19),22-tetraene-1,3,24-triol, (5Z,7E,22E)-(1S,3R,20S,24S)-25-(5-methylthiazol-2-yl)-26,27-cyclo-9,10-secocholesta-5,7,10(19),22-tetraene-1,3,24-triol, (5Z,7E,22E)-(1S,3R,20S,24R)-25-(5-ethylthiazol-2-yl)-26,27-cyclo-9,10-secocholesta-5,7,10(19),22-tetraene-1,3,24-triol, (5Z,7E,22E)-(1S,3R,20S,24S)-25-(5-ethylthiazol-2-yl)-26,27-cyclo-9,10-secocholesta-5,7,10(19),22-tetraene-1,3,24-triol, (5Z,7E,22E)-(1S,3R,20S,24R)-25-(5-butylthiazol-2-yl)-26,27-cyclo-9,10-secocholesta-5,7,10(19),22-tetraene-1,3,24-triol, (5Z,7E,22E)-(1S,3R,20S,24S)-25-(5-butylthiazol-2-yl)-26,27-cyclo-9,10-secocholesta-5,7,10(19),22-tetraene-1,3,24-triol, (5Z,7E,22E)-(1S,3R,20S,24R)-25-(5-pentylthiazol-2-yl)-26,27-cyclo-9,10-secocholesta-5,7,10(19),22-tetraene-1,3,24-triol, (5Z,7E,22E)-(1S,3R,20S,24S)-25-(5-pentylthiazol-2-yl)-26,27-cyclo-9,10-secocholesta-5,7,10(19),22-tetraene-1,3,24-triol, (5Z,7E,22E)-(1S,3R,20S,24R)-25-(4-propyloxazol-2-yl)-26,27-cyclo-9,10-secocholesta-5,7,10(19),22-tetraene-1,3,24-triol, (5Z,7E,22E)-(1S,3R,20S,24S)-25-(4-propyloxazol-2-yl)-26,27-cyclo-9,10-secocholesta-5,7,10(19),22-tetraene-1,3,24-triol, (5Z,7E,22E)-(1S,3R,20S,24R)-25-(4-methyloxazol-2-yl)-26,27-cyclo-9,10-secocholesta-5,7,10(19),22-tetraene-1,3,24-triol, (5Z,7E,22E)-(1S,3R,20S,24S)-25-(4-methyloxazol-2-yl)-26,27-cyclo-9,10-secocholesta-5,7,10(19),22-tetraene-1,3,24-triol, (5Z,7E,22E)-(1S,3R,20S,24R)-25-(4-ethyloxazol-2-yl)-26,27-cyclo-9,10-secocholesta-5,7,10(19),22-tetraene-1,3,24-triol, (5Z,7E,22E)-(1S,3R,20S,24S)-25-(4-ethyloxazol-2-yl)-26,27-cyclo-9,10-secocholesta-5,7,10(19),22-tetraene-1,3,24-triol, (5Z,7E,22E)-(1S,3R,20S,24R)-25-(4-butyloxazol-2-yl)-26,27-cyclo-9,10-secocholesta-5,7,10(19),22-tetraene-1,3,24-triol, (5Z,7E,22E)-(1S,3R,20S,24S)-25-(4-butyloxazol-2-yl)-26,27-cyclo-9,10-secocholesta-5,7,10(19),22-tetraene-1,3,24-triol, (5Z,7E,22E)-(1S,3R,20S,24R)-25-(4-pentyloxazol-2-yl)-26,27-cyclo-9,10-secocholesta-5,7,10(19),22-tetraene-1,3,24-triol, (5Z,7E,22E)-(1S,3R,20S,24S)-25-(4-pentyloxazol-2-yl)-26,27-cyclo-9,10-secocholesta-5,7,10(19),22-tetraene-1,3,24-triol, (5Z,7E,22E)-(1S,3R,20S,24R)-25-(4-propylthiazol-2-yl)-26,27-cyclo-9,10-secocholesta-5,7,10(19),22-tetraene-1,3,24-triol, (5Z,7E,22E)-(1S,3R,20S,24S)-25-(4-propylthiazol-2-yl)-26,27-cyclo-9,10-secocholesta-5,7,10(19),22-tetraene-1,3,24-triol, (5Z,7E,22E)-(1S,3R,20S,24R)-25-(4-methylthiazol-2-yl)-26,27-cyclo-9,10-secocholesta-5,7,10(19),22-tetraene-1,3,24-triol, (5Z,7E,22E)-(1S,3R,20S,24S)-25-(4-methylthiazol-2-yl)-26,27-cyclo-9,10-secocholesta-5,7,10(19),22-tetraene-1,3,24-triol, (5Z,7E,22E)-(1S,3R,20S,24R)-25-(4-ethylthiazol-2-yl)-26,27-cyclo-9,10-secocholesta-5,7,10(19),22-tetraene-1,3,24-triol, (5Z,7E,22E)-(1S,3R,20S,24S)-25-(4-ethylthiazol-2-yl)-26,27-cyclo-9,10-secocholesta-5,7,10(19),22-tetraene-1,3,24-triol, (5Z,7E,22E)-(1S,3R,20S,24R)-25-(4-butylthiazol-2-yl)-26,27-cyclo-9,10-secocholesta-5,7,10(19),22-tetraene-1,3,24-triol, (5Z,7E,22E)-(1S,3R,20S,24S)-25-(4-butylthiazol-2-yl)-26,27-cyclo-9,10-secocholesta-5,7,10(19),22-tetraene-1,3,24-triol, (5Z,7E,22E)-(1S,3R,20S,24R)-25-(4-pentylthiazol-2-yl)-26,27-cyclo-9,10-secocholesta-5,7,10(19),22-tetraene-1,3,24-triol, (5Z,7E,22E)-(1S,3R,20S,24S)-25-(4-pentylthiazol-2-yl)-26,27-cyclo-9,10-secocholesta-5,7,10(19),22-tetraene-1,3,24-triol, (5Z,7E,22E)-(1S,3R,24R)-20-methyl-25-(5-propyloxazol-2-yl)-26,27-cyclo-9,10-secocholesta-5,7,10(19),22-tetraene-1,3,24-triol, (5Z,7E,22E)-(1S,3R,24S)-20-methyl-25-(5-propyloxazol-2-yl)-26,27-cyclo-9,10-secocholesta-5,7,10(19),22-tetraene-1,3,24-triol, (5Z,7E,22E)-(1S,3R,24R)-20-methyl-25-(5-methyloxazol-2-yl)-26,27-cyclo-9,10-secocholesta-5,7,10(19),22-tetraene-1,3,24-triol, (5Z,7E,22E)-(1S,3R,24S)-20-methyl-25-(5-methyloxazol-2-yl)-26,27-cyclo-9,10-secocholesta-5,7, 10(19),22-tetraene-1,3,24-triol, (5Z,7E,22E)-(1S,3R,24R)-25-(5-ethyloxazol-2-yl)-20-methyl-26,27-cyclo-9,10-secocholesta-5,7,10(19),22-tetraene-1,3,24-triol, (5Z,7E,22E)-(1S,3R,24S)-25-(5-ethyloxazol-2-yl)-20-methyl-26,27-cyclo-9,10-secocholesta-5,7,10(19),22-tetraene-1,3,24-triol, (5Z,7E,22E)-(1S,3R,24R)-25-(5-butyloxazol-2-yl)-20-methyl-26,27-cyclo-9,10-secocholesta-5,7,10(19),22-tetraene-1,3,24-triol, (5Z,7E,22E)-(1S,3R,24S)-25-(5-butyloxazol-2-yl)-20-methyl-26,27-cyclo-9,10-secocholesta-5,7,10(19),22-tetraene-1,3,24-triol, (5Z,7E,22E)-(1S,3R,24R)-20-methyl-25-(5-pentyloxazol-2-yl)-26,27-cyclo-9,10-secocholesta-5,7,10(19),22-tetraene-1,3,24-triol, (5Z,7E,22E)-(1S,3R,24S)-20-methyl-25-(5-pentyloxazol-2-yl)-26,27-cyclo-9,10-secocholesta-5,7,10(19),22-tetraene-1,3,24-triol, (5Z,7E,22E)-(1S,3R,24R)-20-methyl-25-(5-propylthiazol-2-yl)-26,27-cyclo-9,10-secocholesta-5,7,10(19),22-tetraene-1,3,24-triol, (5Z,7E,22E)-(1S,3R,24S)-20-methyl-25-(5-propylthiazol-2-yl)-26,27-cyclo-9,10-secocholesta-5,7,10(19),22-tetraene-1,3,24-triol, (5Z,7E,22E)-(1S,3R,24R)-20-methyl-25-(5-methylthiazol-2-yl)-26,27-cyclo-9,10-secocholesta-5,7,10(19),22-tetraene-1,3,24-triol, (5Z,7E,22E)-(1S,3R,24S)-20-methyl-25-(5-methylthiazol-2-yl)-26,27-cyclo-9,10-secocholesta-5,7,10(19),22-tetraene-1,3,24-triol, (5Z,7E,22E)-(1S,3R,24R)-25-(5-ethylthiazol-2-yl)-20-methyl-26,27-cyclo-9,10-secocholesta-5,7,10(19),22-tetraene-1,3,24-triol, (5Z,7E,22E)-(1S,3R,24S)-25-(5-ethylthiazol-2-yl)-20-methyl-26,27-cyclo-9,10-secocholesta-5,7,10(19),22-tetraene-1,3,24-triol, (5Z,7E,22E)-(1S,3R,24R)-25-(5-butylthiazol-2-yl)-20-methyl-26,27-cyclo-9,10-secocholesta-5,7,10(19),22-tetraene-1,3,24-triol, (5Z,7E,22E)-(1S,3R,24S)-25-(5-butylthiazol-2-yl)-20-methyl-26,27-cyclo-9,10-secocholesta-5,7,10(19),22-tetraene-1,3,24-triol, (5Z,7E,22E)-(1S,3R,24R)-25-(5-pentylthiazol-2-yl)-20-methyl-26,27-cyclo-9,10-secocholesta-5,7,10(19),22-tetraene-1,3,24-triol, (5Z,7E,22E)-(1S,3R,24S)-25-(5-pentylthiazol-2-yl)-20-methyl-26,27-cyclo-9,10-secocholesta-5,7,10(19),22-tetraene-1,3,24-triol, (5Z,7E,22E)-(1S,3R,24R)-20-methyl-25-(4-propyloxazol-2-yl)-26,27-cyclo-9,10-secocholesta-5,7,10(19),22-tetraene-1,3,24-triol, (5Z,7E,22E)-(1S,3R,24S)-20-methyl-25-(4-propyloxazol-2-yl)-26,27-cyclo-9,10-secocholesta-5,7,10(19),22-tetraene-1,3,24-triol, (5Z,7E,22E)-(1S,3R,24R)-20-methyl-25-(4-methyloxazol-2-yl)-26,27-cyclo-9,10-secocholesta-5,7,10(19),22-tetraene-1,3,24-triol, (5Z,7E,22E)-(1S,3R,24S)-20-methyl-25-(4-methyloxazol-2-yl)-26,27-cyclo-9,10-secocholesta-5,7,10(19),22-tetraene-1,3,24-triol, (5Z,7E,22E)-(1S,3R,24R)-25-(4-ethyloxazol-2-yl)-20-methyl-26,27-cyclo-9,10-secocholesta-5,7,10(19),22-tetraene-1,3,24-triol, (5Z,7E,22E)-(1S,3R,24S)-25-(4-ethyloxazol-2-yl)-20-methyl-26,27-cyclo-9,10-secocholesta-5,7,10(19),22-tetraene-1,3,24-triol, (5Z,7E,22E)-(1S,3R,24R)-25-(4-butyloxazol-2-yl)-20-methyl-26,27-cyclo-9,10-secocholesta-5,7,10(19),22-tetraene-1,3,24-triol, (5Z,7E,22E)-(1S,3R,24S)-25-(4-butyloxazol-2-yl)-20-methyl-26,27-cyclo-9,10-secocholesta-5,7,10(19),22-tetraene-1,3,24-triol, (5Z,7E,22E)-(1S,3R,24R)-20-methyl-25-(4-pentyloxazol-2-yl)-26,27-cyclo-9,10-secocholesta-5,7,10(19),22-tetraene-1,3,24-triol, (5Z,7E,22E)-(1S,3R,24S)-20-methyl-25-(4-pentyloxazol-2-yl)-26,27-cyclo-9,10-secocholesta-5,7,10(19),22-tetraene-1,3,24-triol, (5Z,7E,22E)-(1S,3R,24R)-20-methyl-25-(4-propylthiazol-2-yl)-26,27-cyclo-9,10-secocholesta-5,7,10(19),22-tetraene-1,3,24-triol, (5Z,7E,22E)-(1S,3R,24S)-20-methyl-25-(4-propylthiazol-2-yl)-26,27-cyclo-9,10-secocholesta-5,7,10(19),22-tetraene-1,3,24-triol, (5Z,7E,22E)-(1S,3R,24R)-20-methyl-25-(4-methylthiazol-2-yl)-26,27-cyclo-9,10-secocholesta-5,7,10(19),22-tetraene-1,3,24-triol, (5Z,7E,22E)-(1S,3R,24S)-20-methyl-25-(4-methylthiazol-2-yl)-26,27-cyclo-9,10-secocholesta-5,7,10(19),22-tetraene-1,3,24-triol, (5Z,7E,22E)-(1S,3R,24R)-25-(4-ethylthiazol-2-yl)-20-methyl-26,27-cyclo-9,10-secocholesta-5,7,10(19),22-tetraene-1,3,24-triol, (5Z,7E,22E)-(1S,3R,24S)-25-(4-ethylthiazol-2-yl)-20-methyl-26,27-cyclo-9,10-secocholesta-5,7,10(19),22-tetraene-1,3,24-triol, (5Z,7E,22E)-(1S,3R,24R)-25-(4-butylthiazol-2-yl)-20-methyl-26,27-cyclo-9,10-secocholesta-5,7,10(19),22-tetraene-1,3,24-triol, (5Z,7E,22E)-(1S,3R,24S)-25-(4-butylthiazol-2-yl)-20-methyl-26,27-cyclo-9,10-secocholesta-5,7,10(19),22-tetraene-1,3,24-triol, (5Z,7E,22E)-(1S,3R,24R)-25-(4-pentylthiazol-2-yl)-20-methyl-26,27-cyclo-9,10-secocholesta-5,7,10(19),22-tetraene-1,3,24-triol, and (5Z,7E,22E)-(1S,3R,24S)-25-(4-pentylthiazol-2-yl)-20-methyl-26,27-cyclo-9,10-secocholesta-5,7,10(19),22-tetraene-1,3,24-triol. Analogs of calcitriol are described in U.S. Pat. Nos. 4,248,791, 4,279,826, 4,305,880, 4,358,406, 4,391,802, 4,717,721, 4,851,401, 4,866,048, 4,897,388, 5,120,722, 5,145,846, 5,411,949, 5,789,399, 5,952,317, 5,976,784, 6,103,709, 6,482,812, 6,503,893, 6,521,608, 7,211,680, 6,441,207, 6,410,523, and 6,399,797, and in U.S. Patent Publication Nos. 2002/0049344, 2003/0018194, and 2005/0209203.

Celastrol

Celastrol, an anti-oxidant and anti-inflammatory agent, is described in U.S. Pat. No. 4,328,309. Celastrol analogs are represented by formulas (II)-(IV):

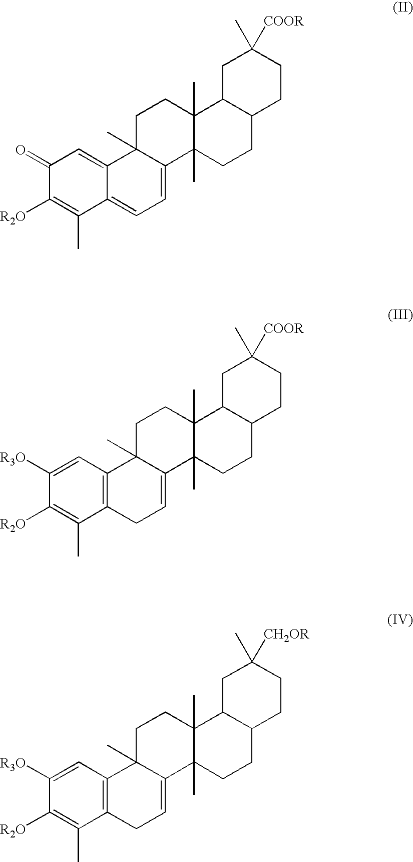

wherein R represents hydrogen, C₁-C₆ alkyl, acetyl, or benzyl; and each of R₂ and R₃ represents independently hydrogen, acetyl, or C₁-C₆ alkyl. Representative celastrol analogs are celastrol methyl ester, celastrol menzyl ester, celastrol butyl ester, dihydrocelastrol, pristimerol, dihydrocelastrol diacetate, pristimerol diacetate, and celastrol triacetate.

Chlorzoxazone

Analogs of chlorzoxazone (also referred to as clorzoxazone), a muscle relaxant, are 2-oxazolidone, cycloserine, 5-chloromethyl-2-oxazolidinone, 4-isopropyl-2-oxazolidinone, 2-benzoisoxazolinone, 4-methyl-5-phenyl-2-oxazolidinone, 4-benzyl-2-oxazolidinone, 5,5-dimethyl oxazolidine-2,4-dione, quinine, carisprodol, cyclobenzaprine, zoxazolamine, benzazoles, 2-(4-dimethylaminophenyl)benzothiazole, 2-(4-diethylaminophenyl)benzothiazole, 2-(2-aminophenyl)benzothiazole, 2-(2-fluorophenyl)benzothiazole, 2-(4-aminobenzyl)benzothiazole, 2-(4-pyridyl)benzothiazole, 2-[4-(pyrrolidin-1-yl)phenyl]benzothiazole, 2-(2-chloro-4-nitrophenyl)benzothiazole, 6-methoxy-2-(4-nitrophenyl)benzothiazole, 2-(2-chloro-4-nitrophenyl)-6-methoxybenzothiazole, 2-(2-chloro-4-nitrophenyl)-7-methoxybenzothiazole, 2-(2-chloro-4-nitrophenyl)-4-methoxybenzothiazole, 2-(4-amino-2-chlorophenyl)-4-methoxybenzothiazole, 2-(4-amino-2-chlorophenyl)-5-methoxybenzothiazole, 2-(4-amino-2-chlorophenyl)-6-methoxybenzothiazole, 2-(4-amino-2-chlorophenyl)-7-methoxybenzothiazole, ethanesulphonic acid salt of 2-(4-aminophenyl)-benzothiazole, and 2-(4-aminophenyl)benzothiazole methanesulphonic acid salt. Chlorzoxazone analogs are described in U.S. Pat. No. 2,895,877.

Dehydroepiandrosterone

Analogs of dehydroepiandrosterone (also referred to as prasterone), a steroid, are dehydroepiandrosterone-sulfate (DHEA-S), the free alcohol of DHEA, DHEA 3-acetate (3-hydroxy-5-androsten-17-one-acetate), DHEA-3-glucuronide (3-hydroxy-5-androsten-17-one-3-glucuronide), DHEA-hemisuccinate, DHEA-valerate, DHEA-enanthate, DHEA-fatty acid derivatives, 16-fluorinated DHEA, 16-brominated DHEA, 7-oxo-DHEA, 7-oxo-DHEA-S, iso-androsterone, etiocholanolone, progesterone, and pregnenolone. Dehydroepiandrosterone analogs are described in U.S. Pat. No. 6,093,706, U.S. Pat. No. 5,824,313, U.S. Pat. No. 5,562,910, U.S. Pat. No. 5,550,120, U.S. Pat. No. 5,518,725, and U.S. Pat. No. 4,496,556.

Diminazene

Analogs of diminazene (also referred to as Berenil), an anti-infective and DNA groove binder, are di-(4-amidino-phenyl)-triazene-(N-1.3), HOE 15 030, N-(3-hydroxypropyl)-Berenil, isometamidium chloride (samorin), ethidium bromide, 7-methyl-10,11-methylenedioxy-20(S)-camptothecin, 7-ethyl-10,11-methylenedioxy-20(S)-camptothecin, 7-ethyl-9-amino-10,11-methylenedioxy-20(S)-camptothecin, 7-ethyl-9-nitro-10,11-methylenedioxy-20(S)-camptothecin, 7-ethyl-10-nitro-20(S)-camptothecin, 7-ethyl-10-amino-20(S)-camptothecin, 7-ethyl-20(S)-camptothecin, 7-propyl-20(S)-camptothecin, 7-ethyl-9-amino-20(S)-camptothecin, 7-ethyl-9-nitro-20(S)-camptothecin, 9-amino-10,11-methylenedioxy-20(S)-camptothecin, 9-chloro-10,11-methylenedioxy-20(S)-camptothecin, 10,11-methylenedioxy-20(S)-camptothecin, 9-chloro-20(S)-camptothecin, 10,11-methylenedioxy-20-glycinate-20(S)-camptothecin, 9-amino-20(S)-camptothecin, 10-amino-20(S)-camptothecin, 10-chloro-20(S)-camptothecin, 20(S)-camptothecin, and pyrithidium bromide. Diminazene analogs are described in U.S. Pat. No. 2,838,485.

Divalproex

Analogs of divalproex, an anti-convulsant, are di-n-propylacetic acid, sodium-di-n-propylacetate, ethyl di-n-propylacetate, di-n-propylacetamide, di-n-propylacetyl urea, sodium 2-isopropylvalerate, sodium di-n-butylacetate, 1-(di-n-propylacetyl)-5,5-diphenylhydantoin, and 2-n-propyl-4-hexynoic acid. Divalprex analogs are described in U.S. Pat. No. 3,325,361.

Exemestane

Analogs of exemestane, a steroid and aromatase inhibitor, are 2-pyridinyl-1-piperazine, N-hydroxy-2,2-dimethyl-4-[[4-(4-pyridinyloxy)phenyl]sulfonyl]-3-thiomorpholinecarboxamide, abarelix, abiraterone acetate, aminoglutethimide, anastrozole, Asta Medica AN-207, antide, AG-041R, avorelin, aseranox, Sensus B2036-PEG, bicalutamide, buserelin, BTG CB-7598, BTG CB-7630, casodex, cetrolix, clastroban, clodronate disodium, cosudex, CR-1505, cytadren, crinone, deslorelin, droloxifene, dutasteride, elimina, EM-800, EM-652, epitiostanol, epristeride, EP-23904, 2-ME, fadrozole, finasteride, flutamide, formestane, FCE-24304, ganirelix, goserelin, Shire gonadorelin agonist, GW-5638, Hoe-766, NCl hCG, idoxifene, isocordoin, ICI-182780, ICI-118630, J015X, Ag J96, ketanserin, lanreotide, LDI-200, letrozol, leuprolide, leuprorelin, liarozole, lisuride hydrogen maleate, loxiglumide, mepitiostane, leuprorelin, LG-1127, LG-1447, LG-2293, LG-2527, LG-2716, LR-103, Lilly LY-326315, LY-353381-HCl, LY-326391, LY-353381, LY-357489, miproxifene phosphate, MPV-2213ad, MZ-4-71, nafarelin, nilutamide, NKSO 1, octreotide, ORG-31710, ORG-31806, orimeten, orimetene, orimetine, ormeloxifene, osaterone, SKB-105657, OSW-1, PTL-03001, PNU-156765, quinagolide, ramorelix, raloxifene, statin, sandostatin LAR, S-10364, SMT-487, somavert, somatostatin, tamoxifen, tamoxifen methiodide, teverelix, toremifene, triptorelin, TT-232, vapreotide, vorozole, YM-116, Yamanouchi YM-511, YM-55208, YM-53789, ZK-1911703, ZK-230211, and ZD-182780. Exemestane analogs are described in U.S. Pat. No. 7,074,800, U.S. Pat. No. 7,071,335, U.S. Pat. No. 6,916,800, U.S. Pat. No. 6,858,598, and U.S. Pat. No. 6,833,373.

Fasudil

Analogs of fasudil, a Rho kinase inhibitor and Ca²⁺ antagonist, are (S)-hexahydro-1-(4-ethenylisoquinoline-5-sulfonyl)-2-methyl-1H-1,4-diazepine, (S)-hexahydro-4-glycyl-2-methyl-1-(4-methylisoquinoline-5-sulfonyl)-1H-1,4-diazepine, (S)-(+)-2-methyl-1-[(4-methyl-5-isoquinoline)sulfonyl]-homopiperazine, fasudil hydrochloride hemihydrate, fasudil hydrochloride trihydrate, BDM [2,3-butanedione 2-monoxime], ML-7 [1-(5-iodonaphthalene-1-sulphonyl)-1H-hexahydro-1,4-diazepine hydrochloride], ML-9 [1-(5-chloronaphthalene-1-sulfonyl)-1H-hexahydro-1,4-diazepine hydrochloride], wortmannin, H-7 [1-(5-isoquinoline sulphonyl)-2-methylpiperazine dihydro-chloride], W-7-[N-(6-aminohexyl)-5-chloro-1-naphthalenesulfonamide], A-3 [N-(6-aminoethyl)-5-chloro-1-naphthalenesulfonamide], N-{1-[1-(5-isoquinolinesulfonyl)imidazol-4(5)-yl-methyl]-2-(4-phenylpiperazinyl)ethyl}-5-isoquinolinesulfonamide, N-[1-(imidazol-4(5)-yl-methyl)-2-(4-phenylpiperazinyl)ethyl]-5-isoquinoline sulfonamide, N-{1-[1-(5-Isoquinolinesulfonyl)Imidazol-4(5)-yl-methyl]-2-(phenylpiperazinyl)ethyl}-N-methyl-5-isoquinoline sulfonamide, 8-methoxyquinoline-5-(N-benzyl-N-methanesulphonyl)sulphonamide, 8-methoxyquinoline-5-(N-benzyl-N-propyl)sulphonamide, N-(2-aminoethyl)-N-methyl-5-isoquinolinesulfonamide, N-(2-aminoethyl)-N-isopropyl-5-isoquinolinesulfonamide, N-(2-aminoethyl)-N-butyl-5-isoquinolinesulfonamide, N-(2-aminoethyl)-N-hexyl-5-isoquinolinesulfonamide, N-(2-aminoethyl)-N-octyl-5-isoquinolinesulfonamide, N-(2-aminoethyl)-N-benzyl-5-isoquinolinesulfonamide, N-(3-aminopropyl)-N-hexyl-5-isoquinolinesulfonamide, N-(4-aminobutyl)-N-hexyl-5-isoquinolinesulfonamide, N-(5-aminopentyl)-N-hexyl-5-isoquinolinesulfonamide, N-(6-aminohexyl)-N-propyl-5-isoquinolinesulfonamide, N-(2-aminopropyl)-N-hexyl-5-isoquinolinesulfonamide, N-(2-aminobutyl)-N-hexyl-5-isoquinolinesulfonamide, N-(2-aminooctyl)-N-hexyl-5-isoquinolinesulfonamide, N-(2-aminodecyl)-N-propyl-5-isoquinolinesulfonamide, N-(2-aminodecyl)-N-hexyl-5-isoquinolinesulfonamide, N-(2-amino-1-methylpropyl)-N-butyl-5-isoquinolinesulfonamide, N-(2-amino-1-methylethyl)-N-ethyl-5-isoquinolinesulfonamide, N-(1-aminomethyl-2-methylpropyl)-N-propyl-5-isoquinolinesulfonamide, N-(1-aminomethylpentyl)-N-hexyl-5-isoquinolinesulfonamidesulfonamide, N-(3-amino-2-methylpropyl)-N-propyl-5-isoquinolinesulfonamide, N-(4-amino-1-methylbutyl)-N-hexyl-5-isoquinolinesulfonamidesulfonamide, N-(5-aminomethylhexyl)-N-ethyl-5-isoquinolinesulfonamidesulfonamide, N-(4-amino-3-methylbutyl)-N-hexyl-5-isoquinolinesulfonamidesulfonamide, N-(4-amino-1-propylbutyl)-N-hexyl-5-isoquinolinesulfonamidesulfonamide, N-(2-methylaminoethyl)-N-methyl-5-isoquinolinesulfonamide, N-(2-ethylaminoethyl)-N-ethyl-5-isoquinolinesulfonamidesulfonamide, N-(2-butylaminoethyl)-N-hexyl-5-isoquinolinesulfonamidesulfonamide, N-ethyl-N-(2-hexylaminoethyl)-5-isoquinolinesulfonamidesulfonamide, N-(2-hexylaminoethyl)-N-hexyl-5-isoquinolinesulfonamidesulfonamide, N-(2-benzylaminoethyl)-N-benzyl-5-isoquinolinesulfonamidesulfonamide, N-butyl-N-(2-phenylethylaminoethyl)-5-isoquinolinesulfonamide, N-(2-benzylaminoethyl)-N-hexyl-5-isoquinolinesulfonamidesulfonamide, N-(3-hexylaminopropyl)-N-hexyl-5-isoquinolinesulfonamidesulfonamide, N-(6-benzylaminohexyl)-N-pentyl-5-isoquinolinesulfonamidesulfonamide, N-(6-hexylaminohexyl)-N-hexyl-5-isoquinolinesulfonamidesulfonamide, N-(2-ethylaminopropyl)-N-hexyl-5-isoquinolinesulfonamidesulfonamide, N-(2-hexylaminopropyl)-N-hexyl-5-isoquinolinesulfonamidesulfonamide, N-(2-propylaminooctyl)-N-butyl-5-isoquinolinesulfonamidesulfonamide, N-hexyl-N-(2-isopropylamino-1-methylethyl)-5-isoquinolinesulfonamide, N-(4-benzylamino-1-methylbutyl)-N-propyl-5-isoquinolinesulfonamide, N-methyl-N-(6-propylamino-5-methylhexyl)-5-isoquinolinesulfonamide, N-(2-diethylaminoethyl)-N-methyl-5-isoquinolinesulfonamidesulfonamide, N-(2-dimethylaminoethyl)-N-hexyl-5-isoquinolinesulfonamidesulfonamide, N-benzyl-N-(2-dihexylaminoethyl)-5-isoquinolinesulfonamidesulfonamide, N-hexyl-N-(2-piperidinoethyl)-5-isoquinolinesulfonamidesulfonamide, N-hexyl-N-(2-morpholinoethyl)-5-isoquinolinesulfonamidesulfonamide, N-[2-(N-cyclohexyl-N-methylamino)ethyl]-N-ethyl-5-isoquinolinesulfonamide, N-hexyl-N-(2-piperidinopropyl)-5-isoquinolinesulfonamidesulfonamide, N-(2-diethylamino-1-methylethyl)-N-hexyl-5-isoquinolinesulfonamide, N-ethyl-N-(5-piperidinopentyl)-5-isoquinolinesulfonamide, 1-(5-isoquinolinesulfonyl)-4-aminopiperidine, 1-(5-isoquinolinesulfonyl)-4-methylaminopiperidine, 1-(5-isoquinolinesulfonyl)-4-ethylaminopiperidine, 1-(5-isoquinolinesulfonyl)-4-propylaminopiperidine, 1-(5-isoquinolinesulfonyl)-4-isopropylaminopiperidine, 1-(5-isoquinolinesulfonyl)-4-butylaminopiperidine, 1-(5-isoquinolinesulfonyl)-4-hexlaminopiperidine, 1-(5-isoquinolinesulfonyl)-4-phenylaminopiperidine, 1-(5-isoquinolinesulfonyl)-4-(N-hexyl-N-methylamino)piperidine, 1-(5-isoquinolinesulfonyl)-4-benzylaminopiperidine, 1-(5-isoquinolinesulfonyl)-4-phenethylaminopiperidine, 1-(5-isoquinolinesulfonyl)-4-piperidinopiperidine, 1-(5-isoquinolinesulfonyl)-3-aminopiperidine, 1-(5-isoquinolinesulfonyl)-3-methylaminopiperidine, 1-(5-isoquinolinesulfonyl)-3-ethylaminopiperidine, 1-(5-isoquinolinesulfonyl)-3-propylaminopiperidine, 1-(5-isoquinolinesulfonyl)-3-isopropylaminopiperidine, 1-(5-isoquinolinesulfonyl)-3-phenylaminopiperidine, 1-(5-isoquinolinesulfonyl)-3-benzylaminopiperidine, 1-(5-isoquinolinesulfonyl)-3-phenethylaminopiperidine, 1-(5-isoquinolinesulfonyl)-3-hexylaminopiperidine, 1-(5-isoquinolinesulfonyl)-3-piperidinopiperidine, 1-(5-isoquinolinesulfonyl)-3-dimethylaminopiperidine, 1-(5-isoquinolinesulfonyl)-3-amino-2-methylpiperidine, 1-(5-isoquinolinesulfonyl)-3-amino-4-methylpiperidine, 1-(5-isoquinolinesulfonyl)-3-methylaminopyrrolidine, 1-(5-isoquinolinesulfonyl)-3-amino-5-methylpyrrolidine, 1-(5-isoquinolinesulfonyl)-3-aminopyrrolidine, 1-(1-hydroxy-5-isoquinolinesulfonyl)-4-aminopiperidine, 1-(1-hydroxy-5-isoquinolinesulfonyl)-4-methylaminopiperidine, 1-(1-hydroxy-5-isoquinolinesulfonyl)-4-propylaminopiperidine, 1-(1-hydroxy-5-isoquinolinesulfonyl)-4-phenylaminopiperidine, 1-(1-hydroxy-5-isoquinolinesulfonyl)-4-benzylaminopiperidine, 1-(1-hydroxy-5-isoquinolinesulfonyl)-3-aminopiperidine, 1-(1-hydroxy-5-isoqyubikubesykfibtk)-3-propylaminopiperidine, 1-(1-hydroxy-5-isoquinolinesulfonyl)-3-benzylaminopiperidine, 1-(1-hydroxy-5-isoquinolinesulfonyl)-3-phenylaminopiperidine, 1-(1-hydroxy-5-isoquinolinesulfonyl)-3-hexylaminopiperidine, 1-(1-hydroxy-5-isoquinolinesulfonyl)-3-phenethylaminopiperidine, 1-(1-hydroxy-5-isoquinolinesulfonyl)-3-dimethylaminopiperidine, 1-(1-hydroxy-5-isoquinolinesulfonyl)-3-amino-5-methylpiperidine, 1-(1-hydroxy-5-isoquinolinesulfonyl)-3-aminopyrrolidine, 1-(1-hydroxy-5-isoquinolinesulfonyl)-3-methylaminopyrrolidine, 1-(1-chloro-5-isoquinolinesulfonyl)-4-methylaminopiperidine, 1-(1-chloro-5-isoquinolinesulfonyl)-4-benzylaminopipepridine, 1-(1-chloro-5-isoquinolinesulfonyl)-3-aminopiperidine, 1-(1-chloro-5-isoquinolinesulfonyl)-3-ethylaminopiperidine, 1-(1-chloro-5-isoquinolinesulfonyl)-3-hexylaminopiperidine, 1-(1-chloro-5-isoquinolinesulfonyl)-3-dimethylaminopiperidine, 1-(1-chloro-5-isoquinolinesulfonyl)-3-(N-hexyl-N-methylamino)piperidine, 1-(1-chloro-5-isoquinolinesulfonyl)-3-aminopyrrolidine, 1-(1-chloro-5-isoquinolinesulfonyl)-3-methylaminopyrrolidine. Fasudil analogs are described in European patent application EP 187,371, U.S. Pat. No. 6,699,508, U.S. Pat. No. 6,696,480, U.S. Pat. No. 6,423,751, U.S. Pat. No. 6,403,590, U.S. Pat. No. 6,271,224, U.S. Pat. No. 6,153,608, U.S. Pat. No. 5,942,505, U.S. Pat. No. 5,747,507, U.S. Pat. No. 5,733,904, U.S. Pat. No. 5,663,174, U.S. Pat. No. 5,340,811, U.S. Pat. No. 5,326,870, U.S. Pat. No. 5,245,034, U.S. Pat. No. 5,245,034, U.S. Pat. No. 5,244,895, U.S. Pat. No. 5,081,246, U.S. Pat. No. 4,857,301, and U.S. Pat. No. 4,798,897.

Fisetin

Fisetin, flavonoid and antioxidant, is described in U.S. Pat. No. 4,591,600. Fisetin analogs are represented by formula (V):

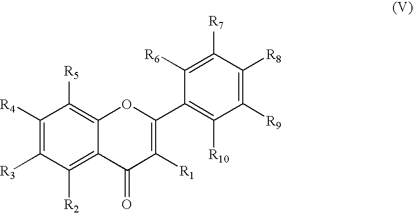

wherein R₁ represents C₁-C₆ alkyl, C₁-C₆ alkoxy, C₅-C₈ cycloalkoxy, methanesulfonyloxy, paratoluenesulfonyloxy, or —CH₂—X—R₁₁, wherein X represents oxygen or sulfur and R₁₁ represents C₁-C₆ alkyl or C₂-C₆ alkenyl; each of R₂-R₅ independently represents hydrogen, hydroxyl, halogen, C₁-C₆ alkyl, or trifluoromethyl, with the proviso that at least one of R₂-R₅ is hydroxyl; and each of R₆-R₁₀ represents hydrogen, hydroxyl, halogen, C₁-C₆ alkyl, C₁-C₄ alkoxy, or trifluoromethyl.

Heat Shock Protein Inhibitor I

Analogs of heat shock protein inhibitor I (also referred to as 3,4-methylenedioxy-benzylidine-γ-butyrolactam or KNK437; Calbiochem. Cat. No. 373260) are KNK423, quercetin, 4-{[3′,4′-(Methylenedioxy)benzyl]amino}-6-methoxyquinazoline, and AP20187. Heat shock protein inhibitor I analogs are described in Koishi et al., Clin. Cancer Res. 7:215-219, 2001, and Yokota et al., Cancer Res. 60:2942-2948, 2000.

Hydroxychloroquine

Hydroxychloroquine, an anti-infective and anti-malarial agent, is described in U.S. Pat. No. 2,546,658. Hydroxychloroquine analogs are represented by formulas (VI)-(VIII): 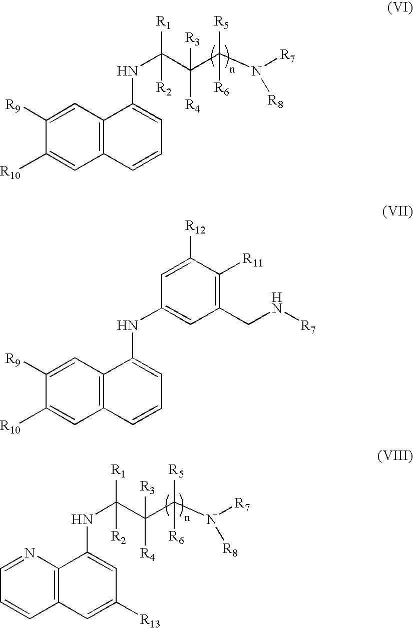
wherein each of R₁ to R₆ represents independently hydrogen or C₁-C₆ alkyl; R₇ and R₈ represent independently C₁-C₆ alkyl, C₂-C₆ alkenyl, C₂-C₆ alkynyl; C₁-C₆ alkaryl, hydroxyalkyl, or together with the nitrogen atom represent pyrrolidine or piperidine, which optionally can be substituted with C₁-C₆ alkyl; R₉ represents hydrogen or halogen; R₁₀ represents hydrogen, halogen, or trifluoromethyl; R₁₁ represents hydrogen or OH; R₁₂ represents hydrogen or parachlorophenyl; and R₁₃ represents hydrogen or methoxyl; and n is an integer between 0 and 3; or R₁ and R₃ represent trimethylene or tetramethylene and n=1; or R₁ and R₇ together represent methylene or dimethylene and n=1; or R₁ and R₇ together represent dimethylene or trimethylene and n=0; or R₃ and R₇ together represent dimethylene or trimethylene and n=1; or R₃ and R₇ together represent trimethylene or tetramethylene and n=0; or R₅ and R₇ together represent trimethylene or tetramethylene and n=1; or R₁ and R₅ together represent dimethylene or trimethylene and n=1. Exemplary hydroxychloroquine analogs are amodiaquine; isopentaquine; pamaquine; pentaquine; tebuquine; primaquine; desethylamodiaquine; desethylhydroxychloroquine; chloroquine; N,N-dideethylchloroquine; N₂-(7-chloro-quinolin-4-yl)-N₁,N₁-dimethyl-ethane-1,2-diamine; N₂-(7-chloro-quinolin-4-yl)-N₁,N₁-diethyl-ethane-1,2-diamine; N₃-(7-chloro-quinolin-4-yl)-N₁,N₁-dimethyl-propane-1,3-diamine; N₃-(7-chloro-quinolin-4-yl)-N₁,N₁-diethyl-propane-1,3-diamine; (7-chloro-quinolin-4-yl)-(2-piperidin-1-yl-ethyl)-amine; (7-chloro-quinolin-4-yl)-[(1-ethyl-pyrrolidin-2-yl)-methyl]-amine; (7-chloro-quinolin-4-yl)-(1-methyl-pyrrolidin-2-yl-methyl)-amine; (7-chloro-quinolin-4-yl)-(1-methyl-piperidin-2-yl-methyl)-amine; (7-chloro-quinolin-4-yl)-(1-methyl-piperidin-3-yl)-amine; (S)—N₂-(7-chloro-quinolin-4-yl)-N₁,N₁-dimethyl-propane-1,2-diamine; (R)—N₂-(7-chloro-quinolin-4-yl)-N₁,N₁-dimethyl-propane-1,2-diamine; N₁-(7-chloro-quinolin-4-yl)-2,N₂,N₂-trimethyl-propane-1,2-diamine; N₃-(7-chloro-quinolin-4-yl)-N₁,N₁-diethyl-propane-1,3-diamine; (RS)-(7-chloro-quinolin-4-yl)-(1-methyl-piperidin-3-yl)-amine; (RS)-(7-chloro-quinolin-4-yl)-(1-methyl-pyrrolidin-3-yl)-amine; (RS)—N₂-(7-chloro-quinolin-4-yl)-N₁,N₁-dimethyl-propane-1,2-diamine; (RS)—N₂-(7-chloro-quinolin-4-yl)-N₁,N₁-diethyl-propane-1,2-diamine; (S)—N₂-(7-chloro-quinolin-4-yl)-N₁,N₁-diethyl-propane-1,2-diamine; (R)—N₂-(7-chloro-quinolin-4-yl)-N₁,N₁-diethyl-propane-1,2-diamine; (RS)-7-chloro-quinolin-4-yl)-(1-methyl-2-pyrrolidin-1-yl-ethyl)-amine; N₂-(7-chloro-quinolin-4-yl)-N₁,N₁-dimethyl-ethane-1,2-diamine; N₂-(7-chloro-quinolin-4-yl)-N₁,N₁-diethyl-ethane-1,2-diamine; N₃-(7-chloro-quinolin-4-yl)-N₁,N₁-dimethyl-propane-1,3-diamine; (R)—N₁-(7-chloro-quinolin-4-yl)-N₂,N₂-dimethyl-propane-1,2-diamine; (S)—N₁-(7-chloro-quinoline-4-yl)-N₂,N₂-dimethyl-propane-1,2-diamine; and (RS)-(7-chloro-quinolin-4-yl)-(1-methyl-pyrrolidin-2-yl-methyl)-amine.

The following compounds that do not satisfy any of formulas (VI)-(VIII) are also hydroxychloroquine analogs: 2-tert-butyl-6-methoxy-8-nitroquinoline; 2-tert-butyl-5,6-dimethoxy-8-nitroquinoline; 2-cyclohexyl-5,6-dimethoxy-8-nitroquinoline; 2-isopropyl-5,6-dimethoxy-8-nitroquinoline; 2-tert-butyl-4-ethyl-5-pentoxy-6-methoxy-8-nitroquinoline; 2-tert-butyl-4-ethyl-5-octoxy-6-methoxy-8-nitroquinoline; 2-tert-butyl-4-methyl-5,6-dimethoxy-8-nitroquinoline; 2-adamantyl-6-methoxy-8-nitroquinoline; 5-cyclopentyl-6-methoxy-8-nitroquinoline and 2,5-dicyclopentyl-6-methoxy-8-nitroquinoline; 5-cyclopentyl-6-methoxy-8-nitroquinoline; 2,5-dicyclopentyl-6-methoxy-8-nitroquinoline; 5-isopropyl-6-methoxy-8-nitroquinoline; 2,5-diisopropyl-6-methoxy-8-nitroquinoline; 5-cyclohexyl-6-methoxy-8-nitroquinoline; 2,5-dicyclohexyl-6-methoxy-8-nitroquinoline; 2-tert-butyl-6-methoxy-8-quinolinamine; 2-adamantyl-6-methoxy-8-quinolinamine; 5-cyclopentyl-6-methoxy-8-quinolinamine; 5-isopropyl-6-methoxy-8-quinolinamine; 5-cyclohexyl-6-methoxy-8-quinolinamine; 2,5-dicyclopentyl-6-methoxy-8-quinolinamine; 2,5-diisopropyl-6-methoxy-8-quinolinamine; 2,5-dicyclohexyl-6-methoxy-8-quinolinamine; 2-tert-butyl-5,6-dimethoxy-8-quinolinamine; 2-cyclohexyl-5,6-dimethoxy-8-quinolinamine; 2-isopropyl-5,6-dimethoxy-8-quinolinamine; 2-tert-butyl-4-ethyl-5-pentoxy-6-methoxy-8-quinolinamine; 2-tert-butyl-4-ethyl-5-octoxy-6-methoxy-8-quinolinamine; 2-tert-butyl-4-methyl-5,6-dimethoxy-8-quinolinamine; 2-[4-(2-tert-butyl-6-methoxy-8-quinolinamino)pentyl]-1,3-isoindolinedione; 2-[4-(5-cyclopentyl-6-methoxy-8-quinylamino)pentyl]-1,3-isoindolinedione; 2-[4-(2-adamantyl-6-methoxy-8-quinolylamino)pentyl]-1,3-isoindolinedione; 2-[4-(5-isopropyl-6-methoxy-8-quinolylamino)pentyl]-1,3-isoindolinedione; 2-[4-(5-cyclohexyl-6-methoxy-8-quinylamino)pentyl]-1,3-isoindolinedione; 2-[4-(2,5-diyclopentyl-6-methoxy-8-quinylamino)pentyl]-1,3-isoindolinedione; 2-[4-(2,5-diisopropyl-6-methoxy-8-quinolylamino)pentyl]-1,3-isoindolinedione; 2-[4-(2,5-dicyclohexyl-6-methoxy-8-quinylamino)pentyl]-1,3-isoindolinedione; 2-[4-(2-tert-butyl-5,6-dimethoxy-8-quinolylamino)pentyl]-1,3-isoindolinedione; 2-[4-(2-cyclohexyl-5,6-dimethoxy-8-quinylamino)pentyl]-1,3-isoindolinedione; 2-[4-(2-isopropyl-5,6-dimethoxy-8-quinolylamino)pentyl]-1,3-isoindolinedione; 2-[4-(2-tert-butyl-4-ethyl-6-methoxy-5-pentoxy-quinolin-8-ylamino)-pentyl]-isoindole-1,3-dione; 2-[4-(2-tert-butyl-4-ethyl-6-methoxy-5-octoxy-quinolin-8-ylamino)-pentyl]-isoindole-1,3-dione; 2-[4-(2-tert-butyl-5,6-methoxy-4-methyl-quinolin-8-ylamino)-pentyl]-isoindole-1,3-dione; N⁸-(4-amino-1-methylbutyl)-2-tert-butyl-6-methoxy-8-quinolinamine; N⁸-(4-amino-1-methylbutyl)-5-cyclopentyl-6-methoxy-8-quinolinamine; N⁸-[4-amino-1-methylbutyl)-2-adamantyl-6-methoxy-8-quinolinamine; N⁸-(4-amino-1-methylbutyl)-5-isopropyl-6-methoxy-8-quinolinamine; N⁸-(4-amino-1-methylbutyl)-5-cyclohexyl-6-methoxy-8-quinolinamine; N⁸-(4-amino-1-methylbutyl)-2,5-dicyclopentyl-6-methoxy-8-quinolinamine; N⁸-(4-amino-1-methylbutyl)-2,5-diisopropyl-6-methoxy-8-quinolinamine; N⁸-(4-amino-1-methylbutyl)-2,5-dicyclohexyl-6-methoxy-8-quinolinamine; N⁸-(4-amino-1-methylbutyl)-2-tert-butyl-5,6-dimethoxy-8-quinolinamine; N⁸-(4-amino-1-methylbutyl)-2-cyclohexyl-5,6-dimethoxy-8-quinolinamine; N⁸-(4-amino-1-methylbutyl)-2-isopropyl-5,6-dimethoxy-8-quinolinamine; N⁸-(4-amino-1-methylbutyl)-2-tert-butyl-4-ethyl-6-methoxy-5-pentoxy-8-quinolinamine; N⁸-(4-amino-1-methylbutyl)-2-tert-butyl-4-ethyl-6-methoxy-5-octoxy-8-quinolinamine; N⁸-(4-amino-3-methylbutyl)-2-tert-butyl-5,6-methoxy-4-methyl-8-quinolinamine; {4-benzyloxycarbonylamino-4-[2-tert-butyl-6-methoxy-quinolin-8-ylamino)-pentyl-carbamoyl]-butyl}-carbamic acid benzyl ester; {1-4-[2-tert-butyl-6-methoxy-quinolin-8-ylamino)-pentylcarbamoyl]-ethyl}-carbamic acid benzyl ester; {5-benzyloxycarbonylamino-5-[4-(2-tert-butyl-6-methoxy-quinolin-8-ylamino)-pentyl-carbamoyl]-pentyl}-carbamic acid benzyl ester; {1-[4-(2-tert-butyl-6-methoxy-8-ylamino)-pentylcarbamoyl]-2-methyl-propyl}-carbamic acid tert-butyl ester; N′-[4-(2-tert-butyl-6-methoxy-8-quinolylamino)pentyl]-(2S)-2,5-diaminopentamide; N′-[4-(2-tert-butyl-6-methoxy-8-quinolylamino)pentyl]-(2S)-2-amino-3-methyl-butanamide; N¹-[4-(2-tert-butyl-6-methoxy-8-quinolylamino)pentyl]-(2S)-2-6-diaminohexanamide; N¹-[4-(2-tert-butyl-6-methoxy-8-quinolylamino)pentyl]-(2S)-2-aminopropanamide; 8-(4-aminopentylamino)-6-methoxyquinoline; 8-(4-aminopentylamino)-6-methoxy-2-methylquinoline; 8-(4-aminopentylamino)-6-methoxy-4-methylquinoline; 8-(4-amino-1-methylbutylamino)-6-methoxy-3-methylquinoline; 8-(4-amino-1-methylbutylamino)-6-methoxy-2-trifluoromethyl quinoline; 8-(4-amino-1-methylbutylamino)-6-methoxy-4-(3-trifluoromethylstyryl)quinoline; 8-(4-amino-1-methylbutylamino)-2-ethyl-6-methoxyquinoline; 8-(4-amino-1-methylbutyl)amino-6-methoxy-2-vinylquinoline maleate; 8-(4-amino-1-methylbutylamino)-4-ethyl-6-methoxyquinoline; 8-(4-amino-1-methylbutylamino)-2-(4-chlorobenzyloxy)-6-methoxyquinoline; 2-amino-8-(4-amino-1-methylbutylamino)-6-methoxyquinoline; 8-(4-amino-1-methylbutylamino)-6-methoxy-1-methyl-1,2,3,4-tetrahydroquinoline; 8-(4-amino-1-methylbutylamino)-5-(4-chlorobenzyloxy)-6-methoxyquinoline; 8-(4-amino-1-methylbutylamino)-5-(4-chlorophenoxy)-6-methoxyquinoline; 8-(4-amino-1-methylbutylamino)-5-ethoxy-6-methoxyquinoline; 8-(4-amino-1-methylbutylamino)-5-dimethylamino-6-methoxyquinoline; 8-(4-amino-1-methylbutylamino)-5,6-dimethoxy-4-methylquinoline; 8-(4-amino-1-ethylbutylamino)-5,6-dimethoxy-4-methylquinoline; 8-(4-aminopentylamino)-5,6-dimethoxy-2,4-dimethylquinoline; 8-(4-amino-1-methylbutylamino)-5,6-methylenedioxy-4-methylquinoline; 8-(6-diethylaminohexylamino)-5-fluoro-6-methoxy-4-methylquinoline; 8-(4-amino-1-methylbutylamino)-6-fluoro-4-methylquinoline; 8-[2-hydroxy-2-methyl-3-(2-propylamino)propylamino]-6-methoxy-4-methylquinoline; 8-(5-amino-1-methylpentylamino)-6-methoxy-4-methylquinoline; 8-(6-aminohexylamino)-6-methoxy-4-methylquinoline; 6-methoxy-8-[6-(2-methyl-1-propylamino)hexylamino]-4-methyl-quinoline; 8-(6-diethylaminohexylamino)-6-methoxy-4-methylquinoline; and 8-(3-diethylaminopropylamino)-2,4-dimethyl-6-methoxyquinoline.

Hydroxyurea

Analogs of hydroxyurea, an anti-metabolite and cancer and sickle-cell anemia therapeutic, are N,N-diethylurea, n-butylurea, β-hydroxyethylurea, 1,1-ethylenediurea, ABT-761 [R(+)-N-[3-[5-(4-fluorophenylmethyl)-2-thienyl]-1-methyl-2-propynyl]-N-hydroxyurea], zileuton ((±)-1-(1-Benzo[b]thien-2-ylethyl)-1-hydroxyurea), (−)-N-1-[2-[3-(4,5-dihydro-4(R)-phenyloxazol-2-yl)phenoxy]ethyl]-N-hydroxyurea, (+)-N-1-[2-[3-(4,5-dihydro-4(R)-phenyloxazol-2-yl)-5-fluorophenoxy]ethyl]-N-hydroxyurea, N-1-[2-[3-[4,5-dihydro-4(R)-phenyloxazol-2-yl]-2-fluorophenoxy]ethyl]-N-hydroxyurea, (+)-N-[3-[3-(4,5-dihydro-4(R)-phenyloxazol-2-yl)phenyl]-2-propyn-1-yl]-N-hydroxy-N′-methylurea, (+)-N-[3-[3-[4,5-dihydro-4(R)-(4-fluorophenyl)oxazol-2-yl]phenyl]-2-propyn-1-yl]-N-hydroxyurea, (−)-N-[3-[3-(4,5-dihydro-4(R)-phenyloxazol-2-yl)-2-fluorophenyl]-2-propyn-1-yl]-N-hydroxyurea, N-[4-[3-(4,5-dihydro-5-phenyloxazol-2-yl)phenyl]-3-butyn-2-yl]-N-hydroxyurea, N-(1-benzylindolin-5-yl)methyl-N-hydroxyurea, N-hydroxy-N-{1-(3-methoxybenzyl)indolin-5-yl}methylurea, and N-hydroxy-N-(1-phenylindolin-5-yl)methylurea. Hydroxyurea analogs are described in U.S. Pat. No. 2,705,727.

Imidocarb Dipropionate

Analogs of imidocarb dipropionate, an anti-infective and anti-protozoal agent, are 3,3′-diamidino-carbanilide and 3,3′-di-2-imidazolin-2-yl-carbanilide. Imidocarb dipropionate analogs are described in British patent GB 1,007,334 and U.S. Pat. No. 3,338,917.

Isoliquiritigenin

Analogs of isoliquiritigenin, a flavonoid and anti-oxidant, are 1,3-diphenyl-2-propen-1-one, lichochalcone A, (E)-4-[3-(3,5-di-tert-butylphenyl)-3-oxo-1-propenyl]benzoic acid, (2E)-1-(2,5-dimethoxyphenyl)-3-[4-(dimethylamino)phenyl]-2-methyl-2-propen-1-one, 3′-methyl-3-hydroxychalcone, N-phenylbenzamide, apigenin, 6-[3-(1-adamantyl)-4-hydroxyphenyl]-2-2-naphthalenecarboxylic acid (CD437, AHPN), 6-[3-(3,5-dimethyl-1-adamantyl)-4-methoxyphenyl]-2-naphthoate, 1-(2,2-bis-hydroxymethyl-benzo[1,3]dioxol-5-yl)-3E-(3,4-dimethoxy-5-thiophen-2-yl-phenyl)-propenone, 1-(2,2-bis-hydroxymethyl-benzo[1,3]dioxol-5-yl)-3E-(4-thiophen-2-yl-phenyl)-propenone, 4-[3E-(5-benzo[b]thien-2-yl-2,4-dimethoxyphenyl)-acryloyl]-benzoic acid, 4-[3E-(4-pyrimidin-5-yl-phenyl)-acryloyl]-benzoic acid, 4-[3E-(4-thiazol-2-yl-phenyl)acryloyl]-benzoic acid, 4-[3E-(2,4-dimethoxy-5-thiophen-2-yl-phenyl)-acryloyl]-benzoic acid, 4-[3E-(5-benzo[b]thiophen-2-yl-2,4-dimethoxy-phenyl)-acryloyl]-benzoic acid, 4-[3E-(3,4-dimethoxy-5-thiophen-2-yl-phenyl)-acryloyl]-benzoic acid, 2-[3E-(5-benzo[b]thiophen-2-yl-2,4-dimethoxy-phenyl)-acryloyl]-benzoic acid, sodium salt, 4-[3E-(4-thiophen-2-yl-phenyl)-acryloyl]-benzoic acid, 1-(4-amino-phenyl)-3E-(3,4-dimethoxy-5-thiophen-2-yl-phenyl)-propenone, 1-(4-amino-phenyl)-3E-(5-benzo[b]thiophen-2-yl-2,4-dimethoxy-phenyl)-propenone, (3-{4-[3E-(4-thiophen-2-yl-phenyl)-acryloyl]-phenyl}-ureido)-acetic acid ethyl ester, and (3-[ethoxycarbonylmethylaminocarbonyl]-3-{4-[3E-(3,4-dimethoxy-5-thiophen-2-yl-phenyl)-acryloyl]-phenyl}-ureido)-acetic acid ethyl ester. Isoliquiritigenin analogs are described in U.S. Pat. No. 7,067,694, U.S. Pat. No. 6,939,990, U.S. Pat. No. 6,864,264, and U.S. Pat. No. 4,085,135.

NKH-477

NKH-477 (i.e., 6-(3-dimethylaminopropionyl)forskolin), an adenylyl cyclase activator, is described in U.S. Pat. No. 5,789,439. NKH-477 analogs are represented by formula (IX): 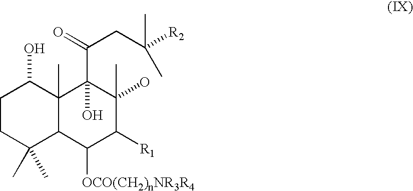
wherein R₁ represents hydrogen or an acetyl group, R₂ represents a hydrocarbon group (i.e., an alkyl, alkenyl, or alkynyl having 2 or 3 carbons, and R₃ and R₄ each represents independently hydrogen or a C₁-C₆ alkyl group or R₃ and R₄ are combined to represent a C₂-C₆ alkenyl group which optionally contains an oxygen or nitrogen atom in the linking chain (e.g., pyrrolidine, piperidine, or morpholine).

Exemplary NKH-477 analogs are 6-(4-dimethylaminobutyryl)forskolin; 6-(5-dimethylaminopentanoyl)forskolin; 6-(6-dimethylaminohexanoyl)forskolin; 6-(3-aminopropionyl)forskolin; 6-(4-aminobutyryl)forskolin; 6-(5-aminopentanoyl)forskolin; 6-(6-aminohexanoyl)forskolin; 14,15-dihydro-6-(3-dimethylaminopropionyl)forskolin; 14,15-dihydro-6-(4-dimethylaminobutyryl)forskolin; 6-(3-dimethylaminopropionyl)-7-deacetylforskolin; 6-(3-N-methylpiperazinopropionyl)-7-deacetylforskolin; 6-(3-piperidinopropionyl)-7-deacetylforskolin; and 6-(3-morpholinopropionyl)-7-deacetylforskolin. NKH-477 analogs are described in U.S. Pat. No. 5,789,439 and EP-A-222413.

PKR Inhibitor

Analogs of PKR inhibitor (also referred to as RNA-dependent protein kinase inhibitor, Calbiochem Cat. No. 527450) are 2-aminopurine, 9-(4-bromo-3,5-dimethyl-pyridin-2-yl)-6-chloro-9H-purin-2-ylamine, 9-(4-bromo-3,5-dimethyl-pyridin-2-ylmethyl)-6-chloro-9H-purin-2-ylamine, phosphate salt, 9-(4-bromo-3,5-dimethyl-pyridin-2-ylmethyl)-6-chloro-9H-purin-2-ylamine, hydrochloric acid salt, 6-bromo-9-(4-bromo-3,5-dimethyl-pyridin-2-ylmethyl)-9H-purin-2-ylamine, 6-bromo-9-(4-bromo-3,5-dimethyl-1-oxy-pyridin-2-yl methyl)-9H-purin-2-ylamine, 2-(2-amino-6-chloro-purin-9-ylmethyl)-3,5-dimethyl-pyridin-4-ol, 9-(4-allyloxy-3,5-dimethyl-pyridin-2-ylmethyl)-6-chloro-9H-purin-2-ylamine, 6-chloro-9-[4-(2-ethoxy-ethoxy)-3,5-dimethyl-pyridin-2-ylmethyl]-9H-purin-2-ylamine, 6-chloro-9-(4-cyclopropylmethoxy-3,5-dimethyl-pyridin-2-ylmethyl)-9H-purin-2-ylamine, 6-chloro-9-(4-isobutoxy-3,5-dimethyl-pyridin-2-ylmethyl)-9H-purin-2-ylamine, 6-chloro-9-(4-chloro-3,5-dimethyl-pyridin-2-ylmethyl)-9H-purin-2-ylamine, 6-chloro-9-(3,5-dimethyl-pyridin-2-yl methyl)-9H-purin-2-ylamine, and 6-bromo-9-(4-methoxy-3,5-dimethyl-pyridin-2-ylmethyl)-9H-purin-2-ylamine, phosphate salt. PKR inhibitor analogs are described in Jammi et al., Biochem. Biophys. Res. Commun. 308:50-57, 2003.

Pyritinol

Analogs of pyritinol, an antioxidant and nootropic, are pyridoxine-5-disulfide, encefabol (or encephabol), bonifen, bonol, biocefalin, 3,3′-(dithiodimethylene)bis[5-hydroxy-6-methyl-4-pyridinemethanol], bis(4-hydroxymethyl-5-hydroxy-6-methyl-3-pyridylmethyl)disulfide, bis[(3-hydroxymethyl-2-methyl-5-pyridyl)methyl]disulfide, dipyridoxolyldisulfide, hematoporphyrin, acetyl-L-carnitine, methylcobalamin, methylcobalamin, glycerylphosphorylcholine, propentofylline, idebenone, pyritinol, piracetam, aniracetam, nefiracetam, oxiracetam, pramiracetam, levetiracetam, hydergine, glutathione, modafinil, centrophenoxine, and galantamine. Pyritinol analogs are described in U.S. Pat. No. 3,010,966.

Quercetin

Analogs of quercetin, a flavonoid and antioxidant, are quercetin pentamethyl carbamate, quercetin chalcone, 3′,4′-((N-carboxymethyl)carbamoyloxy)-3,4′(3′),5,7-tetrahydroxy-flavone, N-methyl-D-glucamine salt, avicularoside, guiajaverin, hyperoside, isohyperoside, isoquercitrin, multinoside A, multinoside A acetate, quercitrin, quercetin-3-O-(2″-O-β-D-glucopyranosyl)-α-L-rhamnopyranoside, quercetin-3-O-(6″-O-galloyl)-glucopyranoside, quercetin-3-O-(6′″-O-p-coumaroyl-β-D-glucopyranosyl-(1-2)-α-L-rhamnopyranoside), quercetin-3-O-D-glucopyranosyl-(1-6)-β-D-glucopyranosyl-(1-4)-α-L-rhamnopyranoside, quercetin-3-O-[2″-O-6′″-O-p-(7″″-O-β-D-glucopyranosyl)coumaroyl-β-D-glucopyranosyl]-α-L-rhamnopyranoside, quercetin-3-O-[6′″-p-coumaroyl-β-D-glucopyranosyl-β-(1-4)-rhamnopyranoside], quercetin-3-O-[α-L-rhamnopyranosyl (1-2)-α-L-rhamnopyranosyl-(1-6)-β-D-glucopyranoside], quercetin-3-O-[-αrhamnopyranosyl (1-4)α-L-rhamnopyranosyl (1-6)β-D-galactopyranoside], quercetin-3-O-[α-rhamnopyranosyl-(1-2)]-[β-glucopyranosyl-(1-6-)]-β-D-galactopyranoside, quercetin-3-O-[α-rhamnopyranosyl-(1-4-)-α-rhamnopyranosyl-(1-6)-β-galactopyranoside], quercetin-3-O-α-L-rhamnopyranosyl-(1-2)-β-D-galactopyranoside, quercetin-3-O-diglucospyranoside, quercetin-3-O-gentiobioside, quercetin-3-O-glucopyranosylgalactopyranoside, quercetin-3-O-neohesperidoside, quercetin-3-gentiotrioside, quercetin-3-methyl ether, quercetin-3-rhamnogentiobioside, quercetin-3-rhamnoglucoside, and quercetin-3-sulfate. Quercetin analogs are described in German patent DE 2,122,514.

Rosiglitazone

Analogs of rosiglitazone, a PPAR agonist and anti-diabetic agent, are (+)-5-[[4-[(3,4-dihydro-6-hydroxy-2,5,7,8-tetramethyl-2H-1-benzopyran-2-yl)methoxy]phenyl]methyl]-2,4-thiazolidinedione, 4-(2-naphthylmethyl)-1,2,3,5-oxathiadiazole-2-oxide, 5-[4-[2-[N-(benzoxazol-2-yl)-N-methylamino]ethoxy]benzyl]-5-methylthiazolidine-2,4-dione, 5-[4-[2-[2,4-dioxo-5-phenylthiazolidin-3-yl)ethoxy]benzyl]thiazolidine-2,4-dione, 5-[4-[2-[N-methyl-N-(phenoxycarbonyl)amino]ethoxy]-benzyl]thiazolidine-2,4-dione, 5-[4-(2-phenoxyethoxy)benzyl]thiazolidine-2,4-dione, 5-[4-[3-(5-methyl-2-phenyloxazol-4-yl)propionyl]benzyl]thiazolidine-2,4-dione, 5-[4-[2-(4-chlorophenyl)ethylsulfonyl]benzyl]thiazolidine-2,4-dione, 5-[4-[3-(5-methyl-2-phenyloxazol-4-yl)propionyl]benzyl]thiazolidine-2,4-dione, 5-(4-[2-(N-methyl-N-(2-pyridyl)amino)-ethoxy]benzyl)2,4-thiazolidinedione, 5-[p-[1-methylcyclohexyl)methoxyl]benzyl]-2,4-thiazolidinedione (ciglitazone), 5-[p-[2-(5-ethyl-2-pyridyl)ethoxy]benzyl]-2,4-thiazolidinedione (pioglitazone), 5-[p-[3-(5-methyl-2-phenyl-4-oxazolyl)propionyl]benzyl]-2,4-thiazolidinedione (darglitazone), and 5-[[(2R)-2-benzyl-6-chromanyl]methyl]-2,4-thiazolidinedione (englitazone). Rosiglitazone analogs are described in U.S. Pat. No. 7,078,428, U.S. Pat. No. 7,071,218, and U.S. Pat. No. 6,911,468.

SU9516

Analogs of SU9516 (also referred to as 3-[1-(3H-imidazol-4-yl)-meth-(Z)-ylidene]-5-methoxy-1,3-dihydro-indol-2-one; Calbiochem Cat. No. 572650), a GSK-3β inhibitor and CDK inhibitor, are 3-[3,5-dimethyl-4-(4-methylpiperazine-1-carbonyl)-1H-pyrrol-2 ylmethylene)-4-pyridin-4-yl-1,3-dihydroindol-2-one, 3-[3-methyl-4-(piperidine-1-carbonyl)-1H-pyrrol-2-ylmethylene]-4-pyridin-4-yl-1,3-dihydroindol-2-one, 3-(3,5-dimethyl-1H-pyrrol-2-ylmethylene)-4-pyridin-4-yl-1,3-dihydroindol-2-one, 3-[2-(2-oxo-4-pyridin-4-yl-1,2-dihydroindol-3-ylidenemethyl-4,5,6,7-tetrahydro-1H-indol-3-yl]-propionic acid, 3-[5-ethyl-2-(2-oxo-4-pyridin-4-yl-1,2-dihydroindol-3-ylidenemethyl)-1H-pyrrol-3-yl]-propionic acid, 4-(2-carboxyethyl)-2-methyl-5-(2-oxo-4-pyridin-4-yl-1,2-dihydro-indol-3-ylidenemethyl)-1H-pyrrole-3-carboxylic acid ethyl ester, 3-[2,4-dimethyl-5-(2-oxo-4-pyridin-4-yl-1,2-dihydroindol-ylidenemethyl)-1H-pyrrol-3-yl]-propionic acid, 4-pyridin-4-yl-3-(4,5,6,7-tetrahydro-1H-indol-2-ylmethylene), 5-methyl-2-(2-oxo-4-pyridin-4-yl-1,2-dihydroindol-ylidenemethyl)-1H-pyrrole-3-carboxylic acid, 3-[3-(3-morpholin-4-yl-propyl)-4,5,6,7-tetrahydro-1H-indol-2 ylmethylene]-4-pyridin-4-yl-1,3-dihydroindol-2-one, 3-[3-methyl-5-(4-methylpiperazine-1-carbonyl)-1H-pyrrol-ylmethylene]-4-pyridin-4-yl-1,3-dihydroindol-2-one, 3-(5-methylthiophen-2-ylmethylene)-4-pyridin-4-yl-1,3-dihydro-indol-2-one, 4-[4-(2-oxo-4-pyridin-4-yl-1,2-dihydroindol-3-ylidenemethyl)phenyl]-piperazine-1-carbaldehyde, 4-(2-hydroxyethyl)-5-2-oxo-4-pyridin-4-yl-1,2-dihydroindol-3-ylidenemethyl)-1H-pyrrole-3-carboxylic acid, [3-methyl-4-(piperidine-1-carbonyl)-1H-pyrrol-2 ylmethylene]-4-piperidin-4-yl-1,3-dihydro-indol-2-one, 3-[3-methyl-4-(morpholine-4-carbonyl)-1H-pyrrolylmethylene]-4-piperidin-4-yl-1,3-dihydroindol-2-one, 3-(3,5-dibromo-4-hydroxy-benzylidine-2-oxo-2,3-dihydro-1H-indole-5-carbonitrile, 3-(3,5-dibromo-4-hydroxy-benzylidene)-5-(2-methyl-thiazol-4-yl)-1,3-dihydro-indol-2-one, 3-(3-bromo-5-ethoxy-4-hydroxy-benzylidene)-5-(2-methyl-thiazol-4-yl)-1,3-dihydro-indol-2-one, 3-(3,5-dichloro-4-hydroxy-benzylidene-5-(2-methyl-thiazol-4-yl)-1,3-dihydro-indol-2-one, (butanoyl)-1,3-dihydro-indol-2-one, 3-(3,5-dibromo-4-hydroxy-benzylidene-5-(3-methyl-butanoyl-1,3-dihydro-indol-2-one, 5-benzoyl-3-(3,5-dibromo-4-hydroxy-benzylidene)-1,3-dihydro-indol-2-one, 5-benzoyl-3-(3,5-dichloro-4-hydroxy-benzylidene)-1,3-dihydro-indol-2-one, 5-benzoyl-3-(3-bromo-5-ethoxy-4-hydroxy-benzylidene)-1,3-dihydro-indol-2-one, 3-(3,5-dichloro-4-hydroxy-benzylidene-5-(3-methyl-butanoyl)-1,3-dihydro-indol-2-one, 3-(3-bromo-5-ethoxy-4-hydroxy-benzylidene)-5-(3-methyl-butanoyl)-1,3-dihydro-indol-2-one, 3-(3,5-dibromo-4-hydroxy-benzylidene-5-(pyridine-3-carbonyl)-1,3-dihydro-indol-2-one, 3-(3,5-dichloro-4-hydroxy-benzylidiene)-5-(pyridine-3-carbonyl)-1,3-dihydro-indol-2-one, 3-(3,5-dibromo-4-hydroxy-benzylidene)-5-(pyridine-4-carbonyl)-1,3-dihydro-indol-2-one, 3-(3-bromo-5-ethoxy-4-hydroxy-benzylidene)-5-(pyridine-4-carbonyl)-1,3-dihydro-indol-2-one, 3-(3,5-dichloro-4-hydroxy-benzylidene)-5-(pyridine-4-carbonyl)-1,3-dihydro-indol-2-one, 3-(3-bromo-5-ethoxy-4-hydroxy-benzylidene)-5-(pyridine-3-carbonyl)-1,3-dihydro-indol-2-one, 3-(3,5-dichloro-4-hydroxy-benzylidene-5-(oxazol-5-yl)-1,3-dihydro-indol-2-one, 3-(3,4-dibromo-4-hydroxy-benzylidene)-5-(oxazol-5-yl)-1,3-dihydro-indol-2-one, 3-(3,5-dibromo-4-hydroxy-benzylidene)-5-(2-ethyl-thiazol-4-yl)-1,3-dihydro-indol-2-one, 3-(3,5-dibromo-4-hydroxy-benzylidene)-2-oxo-2,3-dihydro-1H-indol-5-carboxylic acid methyl ester, 3-(3,5-dibromo-4-hydroxy-benzylidene-5-(furan-2-carbonyl)-1,3-dihydro-indol-2-one, 3-(3,5-dichloro-4-hydroxy-benzylidene-5-(furan-2-carbonyl)-1,3-dihydro-indol-2-one, 3-(3-bromo-5-ethoxy-4-hydroxy-benzylidene-5-(furan-2-carbonyl)-1,3-dihydro-indol-2-one, 5-cyclopropanecarbonyl-3-(3,5-dibromo-4-hydroxy-benzylidene)-1,3-dihydro-indol-2-one, 5-aminomethyl-3-(3,5-dibromo-4-hydroxy-benzylidene)-1,3-dihydro-indol-2-one, 5-cyclopentanecarbonyl-3-(3,5-dibromo-4-hydroxy-benzylidene)-1,3-dihydro-indol-2-one, 3-(3,5-dichloro-4-hydroxy-benzylidene)-2-oxo-2,3-dihydro-1H-indole-5-carboxylic acid methyl ester, 3-(3,5-dibromo-4-hydroxy-benzylidene)-1,3-dihydro-indol-2-one, 3-(3,5-dibromo-4-hydroxy-benzylidene)-5-(thiophene-2-carbonyl)-1,3-dihydro-indol-2-one, 5-(2-amino-thiazol-4-yl)-3-(3,5-dibromo-4-hydroxy-benzylidene)-1,3-dihydro-indol-2-one, 3-(3,5-dibromo-4-hydroxy-benzylidene)-5-(imidazo[1,2a]pyridin-2-yl)-1,3-dihydro-indol-2-one, 3-(3,5-dibromo-4-hydroxy-benzylidene)-5-propionyl-1,3-dihydro-indol-2-one, 3-(3,5-dibromo-4-hydroxy-benzylidene)-2-oxo-2,3-dihydro-1H-indole-5-sulfonic acid amide, 3-(3,5-dibromo-4-hydroxy-benzylidene)-2-oxo-2,3 dihydro-1H-indole-5-sulfonic acid N,N-diethylamide, 3-(3,5-dibromo-4-hydroxy-benzylidene)-5-(pyrrolidine-1-sulfonyl)-1,3-dihydro-indol-2-one, 3-(3,5-dibromo-4-hydroxy-benzylidene)-2-oxo-2,3-dihydro-1H-indol-5-sulfonic acid (N-2-dimethylamino-ethyl)-N-methyl-amide, 3-(3,5-dibromo-4-hydroxy-benzylidene)-5-(isoxazole-5-carbonyl)-1,3-dihydro-indol-2-one, 5-chloro-3-(3,5-dibromo-4-hydroxy-benzylidene)-1,3-dihydro-indol-2-one, 5-chloro-3-(3,5-dichloro-4-hydroxy-benzylidene)-1,3-dihydro-indol-2-one, 3-(3,5-dibromo-4-hydroxy-benzylidene)-5-trifluoromethoxy-1,3-dihydro-indol-2-one, 5-bromo-3-(3,5-dichloro-4-hydroxy-benzylidene)-1,3-dihydro-indol-2-one, 3-(3-bromo-5-ethoxy-4-hydroxy-benzylidene)-5-iodo-1,3-dihydro-indol-2-one, 3-(3-bromo-4-hydroxy-5-methoxy-benzylidene)-5-iodo-1,3-dihydro-indol-2-one, 5-bromo-3-(3,5-diiodo-4-hydroxy-benzylidene)-1,3-dihydro-indol-2-one, 3-(3,5-diiodo-4-hydroxy-benzylidene)-5-trifluoromethoxy-1,3-dihydro-indol-2-one, 3-(3-bromo-4-hydroxy-5-methoxy-benzylidene)-1,3-dihydro-indol-2-one, 3-(3,5-dinitro-4-hydroxy-benzylidene)-1,3-dihydro-indol-2-one, 3-(3,5-dichloro-4-hydroxy-benzylidene)-1,3-dihydro-indol-2-one, 3-(3-chloro-4-hydroxy-5-methoxy-benzylidene)-1,3-dihydro-indol-2-one, 3-(3,5-diiodo-4-hydroxy-benzylidene)-1,3-dihydro-indol-2-one, 3-(3-bromo-4-hydroxy-5-methoxy-benzylidene)-5-chloro-1,3-dihydro-indol-2-one, 5-chloro-3-(3,5-dinitro-4-hydroxy-benzylidene)-1,3-dihydro-indol-2-one, 5-chloro-3-(4-hydroxy-3-methoxy-5-nitro-benzylidene)-1,3-dihydro-indol-2-one, 5-chloro-3-(3-chloro-4-hydroxy-5-methoxy-benzylidene)-1,3-dihydro-indol-2-one, 5-chloro-3-(3,5-diiodo-4-hydroxy-benzylidene)-1,3-dihydro-indol-2-one, 5-bromo-3-(3-bromo-5-ethoxy-4-hydroxy-benzylidene)-1,3-dihydro-indol-2-one, 5-bromo-3-(3-bromo-4-hydroxy-5-methoxy-benzylidene)-1,3-dihydro-indol-2-one, 3-(3-bromo-4-hydroxy-5-methoxy-benzylidene)-5,6-difluoro-1,3-dihydro-indol-2-one, 3-(3-bromo-5-ethoxy-4-hydroxy-benzylidene)-5-trifluoromethoxy-1,3-dihydro-indol-2-one, 3-(3,5-dichloro-4-hydroxy-benzylidene)-5-trifluoromethoxy-1,3-dihydro-indol-2-one, 3-(3-bromo-4-hydroxy-5-methoxy-benzylidene)-5-trifluoromethoxy-1,3-dihydro-indol-2-one, 3-(3,5-dibromo-4-hydroxy-benzylidene)-5,7-dinitro-1,3-dihydro-indol-2-one, 3-(3,5-dibromo-4-hydroxy-benzylidene)-5-nitro-1,3-dihydro-indol-2-one, 3-(3,5-dibromo-4-hydroxy-benzylidene)-7-iodo-1,3-dihydro-indol-2-one, 3-(3,5-dichloro-4-hydroxy-benzylidene)-5-nitro-1,3-dihydro-indol-2-one, 3-(3,5-dibromo-4-hydroxy-benzylidene)-7-iodo-1,3-dihydro-indol-2-one, 7-bromo-3-(3,5-dichloro-4-hydroxy-benzylidene)-1,3-dihydro-indol-2-one, 3-(3-bromo-5-ethoxy-4-hydroxy-benzylidene)-5-nitro-1,3-dihydro-indol-2-one, and 2-(N-{3-[3-(3,5-dibromo-4-hydroxy-benzylidene)-2-oxo-2,3-dihydro-1H-indol-5-yl]-2-oxo-ethyl}-N-methyl-amino)-acetamide. SU9516 analogs are described in Yu et al., Biochem. Pharmacol. 64:1091-1100, 2002, and Lane et al., Cancer Res. 61:6170-6177, 2001.

Tacrine

Analogs of tacrine, a central nervous system agent, are aricept, amirine, SW-10888, MF-217, Ro 45-5934, HP-290, ENA 713, itameline, metrifonate, Tak 177, CP 118.954, galanthamine, ONO 1603, zifrosilone, 6,9-dichloro-1,2,3,4-tetrahydroacridine, 6-chloro-9-fluoro-1,2,3,4-tetrahydroacridine, 1,2-bis-(6-chloro)tacrinyl-ethane, 1,4-bis-(6-chloro)tacrinyl-butane, 1,7-bis-(6-chloro)tacrinyl-heptane, 1,7-bis-(6-fluoro)tacrinyl heptane, 1,8-bis-(6-chloro)tacrinyl-octane, 1,8-bis-(6-fluoro)tacrinyl octane, 1,10-bis-(6-chloro)tacrinyl-decane, 1,4-bis-[(6-chloro-tacrinyl)methyl]-cyclohexane, 1,4-bis-[(6-fluoro-tacrinyl)methyl]-cyclohexane, N-[2-(3-indolyl)ethyl]-6-chlorotacrine, 9-amino-1,2,3,4-tetrahydroacridin-1,2-diol, 9-amino-1,2-dihydroacridin-1,2-diol, 9-amino-1,2,3,4-tetrahydroacridin-3,4-diol, 9-amino-1,2-dihydroacridin-1,2-diol-4(3H)-one, 9-amino-1,4-dihydroxyacridine, 9-amino-2,4-dihydroxyacridine, 9-amino-1,2,3,4-tetrahydroacridin-2,3,4-triol, 9-amino-1,2,3,4-tetrahydroacridin-1,2,3,4-tetraol, and 4-aminoquinoline. Tacrine analogs are described in U.S. Pat. No. 6,254,883, U.S. Pat. No. 6,218,383, U.S. Pat. No. 6,194,403, and U.S. Pat. No. 5,767,126.

Tacrolimus

Tacrolimus, a calcineurin inhibitor, and tacrolimus analogs are described by Tanaka et al., (J. Am. Chem. Soc., 109:5031, 1987), and in U.S. Pat. Nos. 4,894,366, 4,929,611, and 4,956,352. FK506-related compounds, including FR-900520, FR-900523, and FR-900525, are described in U.S. Pat. No. 5,254,562; O-aryl, O-alkyl, O-alkenyl, and O-alkynylmacrolides are described in U.S. Pat. Nos. 5,250,678, 532,248, 5,693,648; amino O-aryl macrolides are described in U.S. Pat. No. 5,262,533; alkylidene macrolides are described in U.S. Pat. No. 5,284,840; N-heteroaryl, N-alkylheteroaryl, N-alkenylheteroaryl, and N-alkynylheteroaryl macrolides are described in U.S. Pat. No. 5,208,241; aminomacrolides and derivatives thereof are described in U.S. Pat. No. 5,208,228; fluoromacrolides are described in U.S. Pat. No. 5,189,042; amino O-alkyl, O-alkenyl, and O-alkynylmacrolides are described in U.S. Pat. No. 5,162,334; and halomacrolides are described in U.S. Pat. No. 5,143,918. All of the above compounds are tacrolimus analogs that can be employed in the methods and compositions of the invention.

Testolactone

Testolactone, an aromatase inhibitor, is described in U.S. Pat. No. 2,744,120. Testolactone analogs are represented by formula (X): 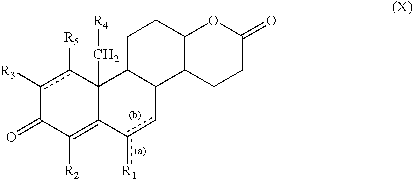
wherein R₁ represents hydrogen or ═CHR₆, wherein R₆ represents hydrogen or C₁-C₆ alkyl and wherein when R₁ is hydrogen, (a) is a single bond and (b) is either a single or double bond while when R₁ is ═CHR₆, (a) is a double bond and (b) is a single bond; R₂ is hydrogen or —OR₇, wherein R₇ is hydrogen, C₁-C₆ alkyl, or a phenyl or benzyl group, each unsubstituted or ring-substituted by one or more substituents selected from C₁-C₄ alkyl, halogen, trifluoromethyl, nitro, amino, hydroxy, and C₁-C₄ alkoxy; R₃ is hydrogen or halogen; R₄ is hydrogen, methyl, or CH₂—S—R₈, wherein R₈ is hydrogen or C₁-C₄ alkyl; R₅ is hydrogen or C₁-C₆ alkyl; and the symbol - - - - indicates the presence of a single or double bond.

Tosufloxacin

Analogs of tosufloxacin, an anti-infective and anti-bacterial agent, are 1-(bicyclo[1.1.1]pent-1-yl)-6,8-difluoro-1,4-dihydro-4-oxo-7-(1-piperazinyl)-3-quinolinecarboxylic acid, 1-(bicyclo[1.1.1]pent-1-yl)-1,4-dihydro-6-fluoro-4-oxo-7-(1-piperazinyl)-3-quinolinecarboxylic acid, 1-(bicyclo[1.1.1]pent-1-yl)-1,4-dihydro-6-fluoro-7-(1-piperazinyl)-4-oxo-3-quinolinecarboxylic acid, 7-[4-(cyclopenten-3-yl)-1-piperazinyl]-1-(bicyclo[1.1.1]pent-1-yl)-1,4-dihydro-6-fluoro-4-oxo-3-quinolinecarboxylic acid, 7-(8-methyl-3,8-diazabicyclo[3.2.1]octan-3-yl)-1-(bicyclo[1.1.1]pent-1-yl)-1,4-dihydro-6-fluoro-4-oxo-3-quinolinecarboxylic acid, 7-[3-phenyl-1-piperazinyl]-1-(bicyclo[1.1.1]pent-1-yl)-1,4-dihydro-6-fluoro-4-oxo-3-quinolinecarboxylic acid, 1-(bicyclo[1.1.1]pent-1-yl)-1,4-dihydro-6-fluoro-7-(4-methyl-1-piperazinyl)-4-oxo-3-quinolinecarboxylic acid, 7-(2,5-diazabicyclo[2.2.2]octan-2-yl)-1-(bicyclo[1.1.1]pent-1-yl)-1,4-dihydro-6-fluoro-4-oxo-3-quinolinecarboxylic acid, 7-(1S,4S-2,5-diazabicyclo[2.2.1]heptan-2-yl)-1-(bicyclo[1.1.1]pent-1-yl)-1,4-dihydro-6-fluoro-4-oxo-3-quinolinecarboxylic acid, 7-(3-(aminomethyl)-1-pyrrolidinyl)-1-(bicyclo[1.1.1]pent-1-yl)-1,4-dihydro-6-fluoro-4-oxo-3-quinolinecarboxylic acid, 7-(3-(ethylamino)methyl-1-pyrrolidinyl-1-(bicyclo[1.1.1]pent-1-yl)-1,4-dihydro-6-fluoro-4-oxo-3-quinolinecarboxylic acid, 7-(3-methyl-1-piperazinyl)-1-(bicyclo[1.1.1]pent-1-yl)-1,4-dihydro-6-fluoro-4-oxo-3-quinolinecarboxylic acid, 7-(3,5-dimethyl-1-piperazinyl)-1-(bicyclo[1.1.1]pent-1-yl)-1,4-dihydro-6-fluoro-4-oxo-3-quinolinecarboxylic acid, 7-(4-(dimethylamino)-1-piperazinyl)-1-(bicyclo[1.1.1]pent-1-yl)-1,4-dihydro-6-fluoro-4-oxo-3-quinolinecarboxylic acid, 7-(4-(1,1-dimethylethyl)-1-piperazinyl)-1-(bicyclo[1.1.1]pent-1-yl)-1,4-dihydro-6-fluoro-4-oxo-3-quinolinecarboxylic acid, 7-(3-(ethylamino)methyl-1-pyrrolidinyl)-1-(bicyclo[1.1.1]pent-1-yl)-1,4-dihydro-6,8-difluoro-4-oxo-3-quinolinecarboxylic acid, 7-(3-amino-1-pyrrolidinyl)-1-(bicyclo[1.1.1]pent-1-yl)-1,4-dihydro-6-fluoro-4-oxo-3-quinolinecarboxylic acid, 7-(3-hydroxy-1-pyrrolidinyl)-1-(bicyclo[1.1.1]pent-1-yl)-1,4-dihydro-6-fluoro-4-oxo-3-quinolinecarboxylic acid, 7-(3-amino-1-pyrrolidinyl)-1-(bicyclo[1.1.1]pent-1-yl)-1,4-dihydro-6-fluoro-4-oxo-3-quinolinecarboxylic acid, 7-(3-amino-4-methyl-pyrrolidin-1-yl)-1-(bicyclo[1.1.1]pent-1-yl)-1,4-dihydro-6-fluoro-4-oxo-3-quinolinecarboxylic acid, 7-(3-hydroxy-1-pyrrolidinyl)-1-(bicyclo[1.1.1]pent-1-yl)-1,4-dihydro-6,8-di fluoro-4-oxo-3-quinolinecarboxylic acid, 7-(3-amino-4-methyl-pyrrolidin-1-yl)-1-(bicyclo[1.1.1]pent-1-yl)-1,4-dihydro-6,8-difluoro-4-oxo-3-quinolinecarboxylic acid, 7-(3-hydroxy-3-methylpyrrolidin-1-yl)-1-(bicyclo[1.1.1]pent-1-yl)-1,4-dihydro-6-fluoro-4-oxo-1,8-naphthyridine-3-carboxylic acid, 7-(3-amino-3-methylpyrrolidin-1-yl)-1-(bicyclo[1.1.1]pent-1-yl)-1,4-dihydro-6-fluoro-4-oxo-1,8-naphthyridine-3-carboxylic acid, 7-(3-hydroxy-3-phenylpyrrolidin-1-yl)-1-(bicyclo[1.1.1]pent-1-yl)-1,4-dihydro-6-fluoro-4-oxo-1,8-naphthyridine-3-carboxylic acid, 7-(3-amino-3-phenylpyrrolidin-1-yl)-1-(bicyclo[1.1.1]pent-1-yl)-1,4-dihydro-6-fluoro-4-oxo-1,8-naphthyridine-3-carboxylic acid, 7-(3,5-dimethyl-1-piperazinyl)-1-(bicyclo[1.1.1]pent-1-yl)-1,4-dihydro-6-fluoro-4-oxo-1,8-naphthyridine-3-carboxylic acid, 7-(3-hydroxy-1-pyrrolidinyl)-1-(bicyclo[1.1.1]pent-1-yl)-1,4-dihydro-6-fluoro-4-oxo-1,8-naphthyridine-3-carboxylic acid, 7-(1-piperazinyl)-1-(bicyclo[1.1.1]pent-1-yl)-1,4-dihydro-6-fluoro-4-oxo-1,8-naphthyridine-3-carboxylic acid, 7-(3-methyl-1-piperazinyl)-1-(bicyclo[1.1.1]pent-1-yl)-1,4-dihydro-6-fluoro-4-oxo-1,8-naphthyridine-3-carboxylic acid, 7-(4-methyl-1-piperazinyl)-1-(bicyclo[1.1.1]pent-1-yl)-1,4-dihydro-6,8-difluoro-4-oxo-3-quinolinecarboxylic acid, 7-(1S,4S-2,5-diazabicyclo[2.2.1]heptan-2-yl)-1-(bicyclo[1.1.1]pent-1-yl)-1,4-dihydro-6-fluoro-4-oxo-1,8-naphthyridine-3-carboxylic acid, 7-(2,5-diazabicyclo[2.2.2octan-2-yl)-1-(bicyclo[1.1.1]pent-1-yl)-1,4-dihydro-6-fluoro-4-oxo-1,8-naphthyridine-3-carboxylic acid, 7-(4-(cyclopenten-3-yl)-1-piperazinyl)-1-(bicyclo[1.1.1]pent-1-yl)-1,4-dihydro-6-fluoro-4-oxo-1,8-naphthyridine-3-carboxylic acid, 7-(3-(ethylamino)methyl-1-pyrrolidinyl)-1-(bicyclo[1.1.1]pent-1-yl)-1,4-dihydro-6-fluoro-4-oxo-1,8-naphthyridine-3-carboxylic acid, 7-(3-methyl-1-piperazinyl)-1-(bicyclo[1.1.1]pent-1-yl)-1,4-dihydro-6-fluoro-4-oxo-1,8-naphthyridine-3-carboxylic acid, 7-(3,4-dimethyl-1-piperazinyl)-1-(bicyclo[1.1.1]pent-1-yl)-1,4-dihydro-6-fluoro-4-oxo-1,8-naphthyridine-3-carboxylic acid, 7-(3-phenyl-1-piperazinyl)-1-(bicyclo[1.1.1]pent-1-yl)-1,4-dihydro-6-fluoro-4-oxo-1,8-naphthyridine-3-carboxylic acid, methanesulfonate, 7-(1R,4R-2,5-diazabicyclo[2.2.1]heptan-2-yl)-1-(bicyclo[1.1.1]pent-1-yl)-1,4-dihydro-6-fluoro-4-oxo-1,8-naphthyridine-3-carboxylic acid, 7-(8-methyl-3,8-diazabicyclo[3.2.1]octan-3-yl)-1-(bicyclo[1.1.1]pent-1-yl)-1,4-dihydro-6-fluoro-4-oxo-1,8-naphthyridine-3-carboxylic acid, 7-(3,8-diazabicyclo[3.2.1]octan-3-yl)-1-(bicyclo[1.1.1]pent-1-yl)-1,4-dihydro-6-fluoro-4-oxo-1,8-naphthyridine-3-carboxylic acid, methanesulfonate, 7-(2-aminomethyl-morpholin-4-yl)-1-(bicyclo[1.1.1]pent-1-yl)-1,4-dihydro-6-fluoro-4-oxo-1,8-naphthyridine-3-carboxylic acid, 7-((S)-3-amino-1-pyrrolidinyl)-1-(bicyclo[1.1.1]pent-1-yl)-1,4-dihydro-6-fluoro-4-oxo-1,8-naphthyridine-3-carboxylic acid, 7-(3-amino-4-methyl-pyrrolidin-1-yl)-1-(bicyclo[1.1.1]pent-1-yl)-1,4-dihydro-6-fluoro-4-oxo-1,8-naphthyridine-3-carboxylic acid, 7-(trans-3-amino-4-methyl-pyrrolidin-1-yl)-1-(bicyclo[1.1.1]pent-1-yl)-1,4-dihydro-6-fluoro-4-oxo-1,8-naphthyridine-3-carboxylic acid, 7-(cis-3-amino-4-methyl-pyrrolidin-1-yl)-1-(bicyclo[1.1.1]pent-1-yl)-1,4-dihydro-6-fluoro-4-oxo-1,8-naphthyridine-3-carboxylic acid, 7-(3-amino-1-pyrrolidinyl)-1-(bicyclo[1.1.1]pent-1-yl)-1,4-dihydro-6-fluoro-4-oxo-1,8-naphthyridine-3-carboxylic acid, 7-(3-amino-1-pyrrolidinyl)-1-(bicyclo[1.1.1]pent-1-yl)-1,4-dihydro-6,8-difluoro-4-oxo-3-quinolinecarboxylic acid, 7-(3-amino-1-pyrrolidinyl)-1-(bicyclo[1.1.1]pent-1-yl)-1,4-dihydro-6-fluoro-4-oxo-1,8-naphthyridine-3-carboxylic acid, 1-cyclopropyl-6-fluoro-7-[8-(methoxyimino)-2,6-diazaspiro[3,4]oct-6-yl]-1,4-dihydro-4-oxo-1,8-naphthyridine-3-carboxylic acid, 1-cyclopropyl-6-fluoro-7-[8-(methoxyimino)-2,6-diazaspiro[3,4]oct-6-yl]-1,4-dihydro-4-oxo-quinoline-3-carboxylic acid, 1-cyclopropyl-6,8-difluoro-7-[8-(methoxyimino)-2,6-diazaspiro[3,4]oct-6-yl]-1,4-dihydro-4-oxo-quinoline-3-carboxylic acid, 1-cyclopropyl-6-fluoro-8-chloro-7-[8-(methoxyimino)-2,6-diazaspiro[3,4]oct-6-yl]-1,4-dihydro-4-oxo-quinoline-3-carboxylic acid, 1-cyclopropyl-5-amino-6,8-difluoro-7-[8-(methoxyimino)-2,6-diazaspiro[3,4]oct-6-yl]-1,4-dihydro-4-oxo-quinoline-3-carboxylic acid, 1-(2,4-difluorophenyl)-6-fluoro-7-[8-(methoxyimino)-2,6-diazaspiro[3,4]oct-6-yl]-1,4-dihydro-4-oxo-quinoline-3-carboxylic acid, 1-(2,4-difluorophenyl)-6-fluoro-7-[8-(methoxyimino)-2,6-diazaspiro[3,4]oct-6-yl]-1,4-dihydro-4-oxo-1,8-naphthyridine-3-carboxylic acid, 1-cyclopropyl-6-fluoro-7-[8-(ethoxyimino)-2,6-diazaspiro[3,4]oct-6-yl]-1,4-dihydro-4-oxo-1,8-naphthyridine-3-carboxylic acid, 1-cyclopropyl-5-amino-6,8-difluoro-7-[8-(ethoxyimino)-2,6-diazaspiro[3,4]oct-6-yl]-1,4-dihydro-4-oxo-quinoline-3-carboxylic acid, 1-cyclopropyl-6-fluoro-7-[8-(methoxyimino)-2-methyl-2,6-diazaspiro[3,4]oct-6-yl]-1,4-dihydro-4-oxo-1,8-naphthyridine-3-carboxylic acid, 1-cyclopropyl-6-fluoro-7-[8-(methoxyimino)-2-methyl-2,6-diazaspiro[3,4]oct-6-yl]-1,4-dihydro-4-oxo-quinoline-3-carboxylic acid, 1-cyclopropyl-6,8-difluoro-7-[8-(methoxyimino)-2-methyl-2,6-diazaspiro[3,4]-oct-6-yl]-1,4-dihydro-4-oxo-quinoline-3-carboxylic acid, 1-cyclopropyl-6-fluoro-8-chloro-7-[8-(methoxyimino)-2-methyl-2,6-diazaspiro[3,4]oct-6-yl]-1,4-dihydro-4-oxo-quinoline-3-carboxylic acid, 1-cyclopropyl-5-amino-6,8-difluoro-7-[8-(methoxyimino)-2-methyl-2,6-diazaspiro[3,4]oct-6-yl]-1,4-dihydro-4-oxo-quinoline-3-carboxylic acid, 2-[4-(1-cyclopropyl-3-carboxy-6,8-difluoro-1,4-dihydro-4-oxo-7-quinolinyl)-1-piperazinyl]2-butenedioic acid dimethyl ester, 2-[4-(1-ethyl-3-carboxy-6-fluoro-1,4-dihydro-4-oxo-7-quinolinyl)-1-piperizinyl]2-butenedioic acid, 2-[4-(1-cyclopropyl-3-carboxy-5,6,8-trifluoro-1,4-dihydro-4-oxo-7-quinolinyl)-1-piperazinyl]2-butenedioic acid, 2-[4-(1-cyclopropyl-3-carboxy-6-fluoro-1,4-dihydro-4-oxo-7-quinolinyl)-1-piperazinyl]2-butenedioic acid, and (E)-2-(N-(3-carboxy-1-cyclopropyl-6,8-difluoro-1,4-dihydro-4-oxo-7-quinolinyl)-1-(3-aminopyrrolinyl))-2-butenedioic acid, 1,4-dimethyl ester. Tosufloxacin analogs are described in German patent DE 3,514,076, Belgium patent BE 904,086, U.S. Pat. No. 7,078,522, U.S. Pat. No. 6,556,196, U.S. Pat. No. 6,313,299, U.S. Pat. No. 5,532,239, U.S. Pat. No. 5,496,947, and U.S. Pat. No. 5,385,906.

Troglitazone

Analogs of troglitazone, a PPAR agonist and anti-diabetic agent, are ((+)-5-[[4-[(3,4-dihydro-6-hydroxy-2,5,7,8-tetramethyl-2H-1-benzopyran-2-yl)-methoxy]phenyl]methyl]-2,4-thiazolidinedione), pioglitazone, rosiglitazone, pentoxifylline, metformin, 5-[4-(6-hydroxy-2,5,7,8-tetramethylchroman-2-ylmethoxy)benzyl]thiazolidine-2,4-dione, 5-[4-(6-hydroxy-2-methyl-7-t-butylchroman-2-ylmethoxy)benzyl]thiazolidine-2,4-dione, 5-[4-(6-hydroxy-2-ethyl-5,7,8-trimethylchroman-2-ylmethoxy)benzyl]-thiazolidine-2,4-dione, 5-[4-(6-hydroxy-2-isobutyl-5,7,8-trimethylchroman-2-ylmethoxy)benzyl]-thiazolidine-2,4-dione, 5-[4-(6-acetoxy-2,5,7,8-tetramethylchroman-2-ylmethoxy)benzyl]thiazolidine-2,4-dione, 5-[4-(6-ethoxycarbonyloxy-2,5,7,8-tetramethylchroman-2-ylmethoxy)benzyl]-thiazolidine-2,4-dione, (+)-5-[[4-[(3,4-dihydro-6-hydroxy-2,5,7,8-tetramethyl-2H-1-benzopyran-2-yl)methoxy]phenyl]methyl]-2,4-thiazolidinedione, 4-(2-naphthylmethyl)-1,2,3,5-oxathiadiazole-2-oxide, 5-[4-[2-[N-(benzoxazol-2-yl)-N-methylamino]ethoxy]benzyl]-5-methylthiazolidine-2,4-dione, 5-[4-[2-[2,4-dioxo-5-phenylthiazolidin-3-yl)-ethoxy]benzyl]thiazolidine-2,4-dione, 5-[4-[2-[N-methyl-N-(phenoxycarbonyl)amino]ethoxy]benzyl]-thiazolidine-2,4-dione, 5-[4-(2-phenoxyethoxy)benzyl]thiazolidine-2,4-dione, 5-[4-[3-(5-methyl-2-phenyloxazol-4-yl)-propionyl]benzyl]thiazolidine-2,4-dione, 5-[4-[2-(4-chlorophenyl)ethylsulfonyl]benzyl]-thiazolidine-2,4-dione, 5-[4-[3-(5-methyl-2-phenyloxazol-4-yl)-propionyl]benzyl]thiazolidine-2,4-dione, 5-(4-[2-(N-methyl-N-(2-pyridyl)amino)-ethoxy]benzyl)2,4-thiazolidinedione, 5-[3-[3-methoxy-4-(5-methyl-2-phenyl-4-oxazolylmethoxy)-phenyl]propyl]-2,4-thiazolidinedione, 5-[3-[3-fluoro-4-(5-methyl-2-phenyl-4-oxazolylmethoxy)-phenyl]propyl]-2,4-thiazolidinedione, 5-[3-[4-(5-methyl-2-phenyl-4-oxazolylmethoxy)phenyl]-butyl]-2,4-thiazolidinedione, 5-[3-[4-(5-methyl-2-phenyl-4-oxazolylmethoxy)phenyl]-propyl]-2,4-thiazolidinedione, 5-[3-[4-(5-methyl-2-naphthyl-4-oxazolylmethoxy)phenyl]-propyl]-2,4-thiazolidinedione, 4-(2-naphthylmethyl)-1,2,3,5-oxathiadiazole-2-oxide, 5-[4-[2-[N-(benzoxazol-2-yl)-N-methylamino]ethoxy]benzyl]-5-methylthiazolidine-2,4-dione, 5-[4-[2-[2,4-dioxo-5-phenylthiazolidin-3-yl)ethoxy]benzyl]thiazolidine-2,4-dione, 5-[4-[2-[N-methyl-N-(phenoxycarbonyl)amino]ethoxy]benzyl]thiazolidine-2,4-dione, 5-[4-(2-phenoxyethoxy)benzyl]thiazolidine-2,4-dione, 5-[4-[2-(4-chlorophenyl)-ethylsulfonyl]benzyl]thiazolidine-2,4-dione, 5-[4-[3-(5-methyl-2-phenyloxazol-4-yl)propionyl]benzyl]thiazolidine-2,4-dione, 5-[4-[(1-methylcyclohexyl)methoxy]-benzyl]thiadiazolidine-2,4-dione (ciglitazone), 5-[[4-(3-hydroxy-1-methylcyclohexyl)methoxy]benzyl]thiadiazolidine-2,4-dione, 5-[4-[2-(5-methyl-2-phenyloxazol-4-yl)ethoxy]benzyl]thiadizolidione-2,4-dione, 5-[4-[2-(5-ethylpyridin-2-yl)ethoxy]benzyl]thiadiazolidine-2,4-dione, 5-[(2-benzyl-2,3-dihydrobenzopyran)-5-ylmethyl]thiadiazoline-2,4-dione (englitazone), 5-[[2-(2-naphthylmethyl)benzoxazol]-5-ylmethyl]thiadiazoline-2,4-dione, 5-[4-[2-(3-phenylureido)ethoxy]benzyl]thiadiazoline-2,4-dione, 5-[4-[2-[N-(benzoxazol-2-yl)-N-methylamino]ethoxy]benzy]thiadiazoline-2,4-dione, 5-[4-[3-(5-methyl-2-phenyloxazol-4-yl)propionyl]benzyl]thiadiazoline-2,4-dione, 5-[2-(5-methyl-2-phenyloxazol-4-ylmethyl)benzofuran-5-ylmethyl]-oxazolidine-2,4-dione, 5-[4-[2-[N-methyl-N-(2-pyridyl)amino]ethoxy]benzyl]thiazolidine-2,4-dione, and 5-[4-[2-[N-(benzoxazol-2-yl)-N-methylamino]ethoxy]benzyl]-oxazolidine-2,4-dione. Troglitazone analogs are described in Japan Kokai 85-51,189, U.S. Pat. No. 6,784,199, U.S. Pat. No. 6,046,222, U.S. Pat. No. 5,972,944, U.S. Pat. No. 5,968,960, U.S. Pat. No. 5,874,454, U.S. Pat. No. 5,728,720, U.S. Pat. No. 5,874,454, U.S. Pat. No. 5,728,720, U.S. Pat. No. 5,708,012, U.S. Pat. No. 5,700,820, U.S. Pat. No. 5,602,133, U.S. Pat. No. 5,478,852, U.S. Pat. No. 5,457,109, U.S. Pat. No. 4,798,835, U.S. Pat. No. 4,791,125, U.S. Pat. No. 4,775,687, U.S. Pat. No. 4,725,610, U.S. Pat. No. 4,703,052, and U.S. Pat. No. 4,572,912.

Tyrphostin 46

Analogs of tyrphostin 46, an EGFR inhibitor, are erbstatin, piceatannol, ST-638, tyrphostin A47, tyrphostin AG17 [(3,5-di-tert-butyl-4-hydroxybenzylidene)-malonitrile], tyrphostin 23, tyrphostin 25, tyrphostin AG490, tyrphostin A, tyrphostin A8, tyrphostin A9, tyrphostin A23, tyrphostin A30, tyrphostin A63, tyrphostin A25, tyrphostin A46, tyrphostin A48, tyrphostin AG126, tyrphostin A51, tyrphostin A47, tyrphostin AG370, tyrphostin B42, tyrphostin B48, tyrphostin B44(−), tyrphostin B46, tyrphostin B56, tyrphostin AG825, tyrphostin B50, tyrphostin AG 879, tyrphostin AG 957, tyrphostin AG1288, tyrphostin AG1295, tyrphostin AG1296, tyrphostin AG1433, tyrphostin AG1478, bis-tyrphostin, aminogenistein, butein, daidzein, damnacanthal, emodin, erbstatin analog, geldanamycin, genistein, herbimycin A, lavendustin A, lavendustin B, lavendustin C, lavendustin C methyl ester, and leflunomide. Tyrphostin 46 analogs are described in U.S. Pat. No. 7,070,936, U.S. Pat. No. 7,045,613, and U.S. Pat. No. 7,005,445.

UCH-L1 inhibitor

Analogs of UCH-L1 inhibitor are SCH66336, L778123, BMS-214662, R115777, FTI-277, O-acyl oximes, isatin, menadione 9, and vitamin K3.

Vanadyl

Analogs of vanadyl (e.g., sulfate hydrate salt), a phosphatase inhibitor and insulin signaling modulator, are chromium picolinate, vitamin E, vanadyl acetylacetonate, vanadium pentoxide, vanadium trisulfate, vanadyl chloride, vanadyl glycinate, vanadyl gluconate, vanadyl citrate, vanadyl lactate, vanadyl tartrate, vanadyl gluconate, vanadyl phosphate, sodium orthovanadate, vanadium chelidamate or arginate, vanadyl complexes with monoprotic bidentate 2,4-diones, and vanadyl phthalocyanine. Analogs of vanadyl sulfate are described in U.S. Pat. Nos. 4,882,171, 5,023,358, 5,045,316, 5,527,790, 5,547,685, 5,871,779, and 6,413,946, and U.S. Patent Publication Nos. 2003/0216412 and 2006/0165814.

Zopiclone

Analogs of zopiclone, a hypnotic and sedative, are pyrrolo[3,4-b]pyrazine, 6-(5-chloropyrid-2-yl)-5-(4-methylpiperazin-1-yl)carbonyloxy-7-oxo-5,6-dihydropyrrolo[3,4-b]pyrazine, pyrrolo[3,4-b]pyrazine, 5-(4-methylpiperazin-1-yl)carbonyloxy-6-(3-nitrophenyl)-7-oxo-5,6-dihydropyrrolo[3,4-b]pyrazine, [3,4-b]pyrazine, 5-(4-methylpiperazin-1-yl)carbonyloxy-6-(6-methylpyridazin-3-yl)-7-oxo-5,6-dihydropyrrolo[3,4-b]pyrazine, pyrrolo[3,4-b]pyrazine, 6-(7-chloroquinol-2-yl)-5-(4-methylpiperazin-1-yl)carbonyloxy-7-oxo-5,6-dihydropyrrolo[3,4-b]pyrazine, pyrrolo[3,4-b]pyrazine, 4-[6-(5-chloropyrid-2-yl)-7-oxo-5,6-dihydropyrrolo[3,4-b]-pyrazin-5-yl]oxycarbonyl-1-methylpiperazine-1-oxide, acetyldesmethylzopiclone, N-desmethylzopiclone, carboethoxydesmethylzopiclone, ureadesmethylzopiclone, amidodesmethylzopiclone, methylamidodesmethylzopiclone, formyldesmethylzopiclone, 6-(5-chloropyridin-2-yl)-7-oxo-5-(4-nitrophenyloxycarbonyloxy)-5,6-dihydropyrrolo[3,4b]pyrazine, 6-(5-chloropyridin-2-yl)-7-oxo-5-(2-propenyloxycarbonyloxy)-5,6-dihydropyrrolo[3,4b]pyrazine, 5-(1,1-dimethyl-2,2,2-trichloroethyloxycarbonyloxy)-6-(5-chloropyridin-2-yl)-7-oxo-5,6-dihydropyrrolo[3,4b]pyrazine, 6-(5-chloropyridin-2-yl)-7-oxo-5-(2,2,2-trichloroethyloxycarbonyloxy)-5,6-dihydropyrrolo[3,4b]pyrazine, 5-(2-chloroethyloxycarbonyloxy)-6-(5-chloropyridin-2-yl)-7-oxo-5,6-dihydropyrrolo[3,4b]pyrazine, 5-(1-chloroethyloxycarbonyloxy)-6-(5-chloropyridin-2-yl)-7-oxo-5,6-dihydropyrrolo[3,4b]pyrazine, 5-(chloromethyloxycarbonyloxy)-6-(5-chloropyridin-2-yl)-7-oxo-5,6-dihydropyrrolo[3,4b]pyrazine, 6-(5-chloropyridin-2-yl)-7-oxo-5-(N-succinimidyloxycarbonyloxi)-5,6-dihydropyrrolo[3,4b]pyrazine, midazolam, zolpidem, brotizolam, and triazolam. Zopiclone analogs are described in German patent DE 2,300,491 and U.S. Pat. No. 3,862,149.

Additional Agents

Analogs of 1-(5-isoquinolinesulfonyl)-2-methylpiperazine, a PKC inhibitor and PKA inhibitor, are described in U.S. Pat. No. 7,005,274, U.S. Pat. No. 6,949,565, U.S. Pat. No. 6,815,450, and U.S. Pat. No. 6,153,608. Analogs of 5-methyl-5-6-7-8-tetrahydropteroylglutamic-acid, a DHFS inhibitor, are described in U.S. Pat. Nos. 4,665,176, 5,239,074, 5,300,505, 5,538,734, 5,698,693, and 6,500,829, and in U.S. Patent Publication Nos. 2002/0052374 and 2003/0007961. Analogs of 5,6-dichloro-1-beta-D-ribofuranosylbenzimidazole (DRB), an RNA polymerase II inhibitor, are described in U.S. Pat. No. 7,018,836, U.S. Pat. No. 6,660,750, and U.S. Pat. No. 6,617,171. Analogs of A-134974 (also referred to as N⁷-[(1′R,2′S,3′R,4′S)-2′,3′-dihydroxy-4′-aminocyclopentyl]-4-amino-5-iodopyrrolopyrimidine; Sigma Cat. No. A2846), an adenosine kinase inhibitor, are described in Zhu et al., Brain Res. 905:104-110, 2001, and McGaraughty et al., J. Pharmacol. Exp. Ther., 296:501-509, 2001. Analogs of Aicar, an AMPK activator, are described in U.S. Pat. No. 6,967,193, U.S. Pat. No. 6,946,115, U.S. Pat. No. 6,919,322, U.S. Pat. No. 6,756,360, U.S. Pat. No. 6,752,981, U.S. Pat. No. 6,617,439, and U.S. Pat. No. 6,312,662. Analogs of amlexanox, an antihistamine (H1), leukotriene antagonist, anti-allergic agent, anti-inflammatory agent, and topical agent, are described in Belgium patent BE 864,647, U.S. Pat. No. 4,143,042, U.S. Pat. No. 5,952,338, U.S. Pat. No. 5,420,307, U.S. Pat. No. 4,728,509, U.S. Pat. No. 4,716,167, U.S. Pat. No. 4,539,326, and U.S. Pat. No. 4,302,463. Analogs of androstanolone, a steroid, are described in U.S. Pat. No. 2,927,921. Analogs of benzohydramic acid (also referred to as benzohydroxamic acid) are described in U.S. Pat. No. 6,248,782, U.S. Pat. No. 5,036,157, U.S. Pat. No. 4,942,253, and U.S. Pat. No. 4,859,233. Analogs of bucladesine, a PDE inhibitor and cAMP analog, are described in Japan Kokai 76-113,896,77-39,698, and 77-39,699. Analogs of carbachol hydrochloride, an mAChR agonist and anti-glaucoma agent, are described in German patents DE 539,329, DE 553,148, and DE 590,311. Analogs of chenodeoxycholic acid diacetate methyl ester, a steroid, are described in U.S. Pat. No. 5,349,074, U.S. Pat. No. 4,895,679, U.S. Pat. No. 4,547,271, U.S. Pat. No. 4,425,273, U.S. Pat. No. 4,331,607, U.S. Pat. No. 4,316,849, U.S. Pat. No. 4,316,848, U.S. Pat. No. 4,301,246, U.S. Pat. No. 4,079,133, U.S. Pat. No. 4,022,806, and U.S. Pat. No. 3,965,131. Analogs of chloroquine phosphate, an anti-infective and anti-malarial agent, are described in U.S. Pat. No. 2,233,970. Analogs of chrysin, a flavonoid and anti-oxidant, are described in U.S. Pat. No. 3,155,579. Analogs of clorofene (also referred to as chlorophene), an anti-infective and anti-bacterial agent, are described in German patent DE 703,955 and U.S. Pat. No. 1,967,825. Analogs of digitoxin, a cardiac glycoside and ATPase inhibitor, are described in U.S. Pat. No. 6,380,167, U.S. Pat. No. 5,153,178, U.S. Pat. No. 4,761,417, U.S. Pat. No. 4,436,828, U.S. Pat. No. 4,282,151, and U.S. Pat. No. 4,133,949. Analogs of diphenyleneiodonium, an iNOS inhibitor, are described in U.S. Pat. Nos. 4,623,666, 6,043,268, 6,372,796, 6,375,944, 6,489,308, 6,593,372, and 7,008,630, and in U.S. Patent Publication Nos. 2004/0220242, 2005/0124701, 2005/0209326, and 2007/0082910. Analogs of forskolin (also referred to as colforsin), an AC activator, are described in German patent DE 2,557,784, U.S. Pat. No. 5,610,315, U.S. Pat. No. 5,484,954, U.S. Pat. No. 5,177,207, U.S. Pat. No. 5,145,855, U.S. Pat. No. 5,093,336, U.S. Pat. No. 4,999,351, U.S. Pat. No. 4,978,678, U.S. Pat. No. 4,954,642, and U.S. Pat. No. 4,088,659. Analogs of GSK-3β inhibitor VIII (also referred to as AR-A014418; N-(4-Methoxybenzyl)-N′-(5-nitro-1,3-thiazol-2-yl)urea; Calbiochem. Cat. No. 361549) are described in Martinez et al., J. Med. Chem. 45:1292-1299, 2002. Analogs of indirubin-3′-monooxime, a GSK-3β inhibitor and CDK inhibitor, are described in U.S. Pat. No. 6,933,315 and U.S. Pat. No. 6,566,341. Analogs of kaempferol, a flavonoid and antioxidant, are described in U.S. Pat. Nos. 4,774,229, 5,043,323, 5,478,579, 5,650,433, 6,444,221, 6,555,573, 6,576,271, 6,576,660, and 6,638,543, and in U.S. Patent Publication Nos. 2001/0047032, 2002/0142012, 2002/0165207, 2003/0104082, 2004/0057908, 2004/0171592, 2004/0242503, and 2006/0002914. Analogs of kenpaullone (also referred to as 9-Bromopaullone and NSC-664704), a GSK-3β inhibitor and CDK inhibitor, are described in U.S. Pat. No. 6,949,558. Analogs of maduramicin NH₄, an anti-infective and anti-bacterial agent, are described in U.S. Pat. No. 5,242,814, U.S. Pat. No. 5,100,785, U.S. Pat. No. 5,043,353, U.S. Pat. No. 4,992,423, U.S. Pat. No. 4,278,663, U.S. Pat. No. 4,407,946, U.S. Pat. No. 4,496,549, and U.S. Pat. No. 4,510,134. Analogs of methylglyoxal (also referred to as pyruvaldehyde), an enzyme inhibitor, anti-proliferation agent, and anti-cancer agent, are described in U.S. Pat. No. 6,613,793, U.S. Pat. No. 6,596,755, U.S. Pat. No. 6,214,172, U.S. Pat. No. 4,302,609, U.S. Pat. No. 4,238,500, and U.S. Pat. No. 4,158,019. Analogs of mofebutazone, an anti-inflammatory agent and inhibitor of prostglandin and leukotrienes, are described in British patent GB 839,057. Analogs of narasin, an anti-infective agent, anti-bacterial agent, and coccidiostat, are described in German patent DE 525,095, U.S. Pat. No. 5,047,338, U.S. Pat. No. 4,342,829, U.S. Pat. No. 4,309,504, U.S. Pat. No. 4,204,039, U.S. Pat. No. 4,174,404, U.S. Pat. No. 4,141,907, and U.S. Pat. No. 4,038,384. Analogs of nigericin, an anti-infective agent, anti-bacterial agent, and ionophore, are described in U.S. Pat. No. 3,555,150. Analogs of novobiocin, an anti-infective agent and anti-bacterial agent, are described in U.S. Pat. No. 3,000,873, U.S. Pat. No. 3,049,475, U.S. Pat. No. 3,049,476, U.S. Pat. No. 3,049,534, U.S. Pat. No. 3,068,221, U.S. Pat. No. 2,925,411, U.S. Pat. No. 2,966,484, and U.S. Pat. No. 2,983,723. Analogs of Pefabloc SC, a protease inhibitor, are described in U.S. Pat. Nos. 5,795,917, 5,998,216, and 6,440,938, and in U.S. Patent Publication Nos. 2002/0019325 and 2006/0205671. Analogs of pifithrin-α (also referred to as 2-(2-imino-4,5,6,7-tetrahydrobenzothiazol-3-yl)-1-p-tolylethanone hydrobromide or PFT-α; Sigma Cat. No. P4359), a p53 inhibitor, are described in U.S. Pat. No. 6,593,353, U.S. Pat. No. 6,949,537, U.S. Pat. No. 6,982,277, U.S. Pat. No. 6,998,240, U.S. Pat. No. 7,008,956, and U.S. Pat. No. 7,012,087, and in Komarova et al., Biochemistry 65:41-48, 2000, and Komarov et al., Science 285:1733-1737, 1999. Analogs of pregnenolone (also referred to as 5-pregnen-3β-ol-20-one or 3β-hydroxy-5-pregnen-20-one; Sigma Cat. No. P9129), a steroid and memory and immune system enhancer, are described in Swiss patent 215,139 and in U.S. Pat. No. 3,963,707, U.S. Pat. No. 4,102,884, U.S. Pat. No. 4,189,400, U.S. Pat. No. 4,220,775, U.S. Pat. No. 4,224,229, U.S. Pat. No. 4,609,496, U.S. Pat. No. 5,391,776, U.S. Pat. No. 5,866,603, U.S. Pat. No. 6,967,194, and U.S. Pat. No. 7,060,290. Analogs of pyruvate, a vitamin and nutrient, are described in U.S. Pat. No. 6,943,190, U.S. Pat. No. 6,916,850, and U.S. Pat. No. 6,900,218. Analogs of salinomycin, an anti-infective agent, anti-bacterial agent, and ionophore, are described in Japan Kokai 72-25392, German patent DE 2,253,031, and U.S. Pat. No. 3,857,948. Analogs of SB-415286, a GSK-3β inhibitor and CDK inhibitor, are described in U.S. Pat. No. 6,780,625 and U.S. Pat. No. 6,770,451. Analogs of spironolactone, a steroid, are described in U.S. Pat. No. 3,013,012. Analogs of tetrahydropapaveroline hydrobromide (also referred to as papaveroline), a dopamine metabolite, vasodilator, and PDE inhibitor, are described in U.S. Pat. No. 6,869,974, U.S. Pat. No. 6,680,047, U.S. Pat. No. 6,635,274, U.S. Pat. No. 6,555,663, and U.S. Pat. No. 6,472,425. Analogs of TPEN (N,N,N′,N′-tetrakis(2-pyridylmethyl)-ethylenediamine), a heavy metal chelator, are described in U.S. Pat. No. 6,297,374, U.S. Pat. No. 6,022,967, U.S. Pat. No. 5,750,704, U.S. Pat. No. 5,461,167, U.S. Pat. No. 5,428,032, U.S. Pat. No. 5,298,507, U.S. Pat. No. 5,204,360, U.S. Pat. No. 5,001,138, U.S. Pat. No. 4,845,106, and U.S. Pat. No. 4,742,060. Analogs of tranylcypromine, an anti-depressant and non-selective MAO-A/B inhibitor, are described in U.S. Pat. No. 2,997,422. Analogs of UCH-L1 inhibitor are described in U.S. Pat. Nos. 5,192,792, 6,288,089, and 6,838,477, and in U.S. Patent Publication Nos. 2005/0272068 and 2007/0071724. Particularly effective therapeutic agents that may be used in the compositions, methods, and kits of the present invention include GSK-3β inhibitors, CDK inhibitors, PKR inhibitors, EGFR inhibitors, flavonoids, antioxidants, PDE inhibitors, and caspase inhibitors, as described in more detail below.

GSK-3β Inhibitors

Glycogen synthase kinase 3 (GSK-3) proteins are involved in a variety of cellular processes, including cell cycle regulation, axonal outgrowth, and the WNT signaling pathway. High levels of the glycogen synthase kinase GSK-3β are associated with neurodegenerative disorders. Inhibitors of GSK-3β may thus be useful in the present invention to treat neurodegenerative disorders. GSK-3β inhibitors that may be suitable for use in the compositions, kits, and methods of the invention include, e.g., α-4-dibromoacetophenone, 2-chloro-1-(4,5-dibromo-thiophen-2-yl)-ethanone, 2-thio(3-iodobenzyl)-5-(1-pyridyl)-[1,3,4]-oxadiazole, (2′Z,3′E)-6-bromoindirubin-3′-acetoxime, (2′Z,3′E)-6-bromoindirubin-3′-oxime, 3-(1-(3-hydroxypropyl)-1H-pyrrolo[2,3-b]pyridin-3-yl]-4-pyrazin-2-yl-pyrrole-2,5-dione, 3-amino-1H-pyrazolo[3,4-β]quinoxaline, 5-amino-3-((4-(aminosulfonyl)phenyl)amino)-N-(2,6-difluorophenyl)-1H-1,2,4-triazole-1-carbothioamide, (5-methyl-1H-pyrazol-3-yl)-(2-phenylquinazolin-4-yl)amine, AF267B, aloisine A, aloisine RP106, AR-A014418, hymenialdisine, indirubin-3′-monoxime, indirubin-3′-monoxime, 5-iodo-indirubin-3′-monoxime-5-sulphonic acid, isogranulatimide, lithium, OTDZT, paullones (e.g., alsterpaullone, 1-azakenpaullone, kenpaullone, 2-iodopaullone, 7-Bromo-5-(4-nitrophenylhydrazono)-4,5-dihydro-1H-[1]benzazepin-2(3H)-one, 2-bromo-9-nitropaullone, 7,8-dimethoxy-5-(4-nitrophenylhydrazono)-4,5-dihydro-1H-[1]benzazepin-2(3H)-one, 2,3-dimethoxy-9-nitropaullone, and 9-cyano-2,3-dimethoxypaullone), Ro-31-8220TDZD-8, SB-415286, SU9516, TWS119, and analogs thereof. Additional GSK-3β inhibitors are described in U.S. Pat. Nos. 7,056,939, 7,045,519, 7,037,918, 6,989,382, 6,949,547, 6,872,737, 6,800,632, 6,780,625, 6,608,063, 6,489,344, 6,479,490, 6,441,053, 6,417,185, 6,323,029, 6,316,259, and 6,057,117.

CDK Inhibitors

Cyclin-dependent kinases (CDKs) are involved in controlling the cell cycle, apoptosis, neuronal functions and neurodegeneration, transcription, and exocytosis. Inhibitors of CDKs may be used in the present invention to treat disorders associated with abnormal cell cycle regulation, e.g., neurodegenerative disorders. CDK inhibitors that may be suitable for use in the compositions, kits, and methods of the invention include, e.g., 2-(3-Hydroxypropylamino)-6-(o-hydroxybenzylamino)-9-isopropylpurine, 2-bromo-12,13-dihydro-5H-indolo[2,3-a]pyrrolo[3,4-c]carbazole-5,7(6H)-dione, 3-(2-Chloro-3-indolylmethylene)-1,3-dihydroindol-2-one, 2(bis-(Hydroxyethyl)amino)-6-(4-methoxybenzylamino)-9-isopropyl-purine, 3-Amino-1H-pyrazolo[3,4-b]quinoxaline, 5-amino-3-((4-(aminosulfonyl)phenyl)amino)-N-(2,6-difluorophenyl)-1H-1,2,4-triazole-1-carbothioamide, aloisine A, aloisine RP106, alsterpaullone 2-cyanoethyl, alvocidib, aminopurvalanol A, bohemine, CGP74514A, ethyl-(6-hydroxy-4-phenylbenzo[4,5]furo[2,3-b])pyridine-3-carboxylate, fisetin, N⁴-(6-Aminopyrimidin-4-yl)-sulfanilamide, flavopiridol, kenpaullone, NSC 625987, NU6102, NU6140, olomoucine, olomoucine II, roscovitine, SU9516, WR 216174, and analogs thereof. Additional CDK inhibitors are described in U.S. Pat. Nos. 7,084,271, 7,078,591, 7,078,525, 7,074,924, 7,067,661, 6,992,080, 6,939,872, 6,919,341, 6,710,227, 6,683,095, 6,677,345, 6,610,684, 6,593,356, 6,569,878, 6,559,152, 6,531,477, 6,500,846, 6,448,264, and 6,107,305.

Caspase Inhibitors

Caspase inhibitors interfere with the activity of caspases (cysteine aspartyl proteases), which are associated with programmed cell death, or apoptosis. Caspase inhibitors may therefore be useful for treating disorders associated with an increase in cellular apoptosis, e.g., neurodegenerative disorders. Caspase inhibitors that may be suitable for use in the compositions, kits, and methods of the invention include, e.g., BOC-D-FMK, CrmA, CV1153, EI15071, EI15072, IDN1965, IDN5370, IDN6734, L709049, L826791, LB84451, MX1122, p35, PF03491390, Q-VD-OPH, SDZ224015, TBC4521, VE13045, VE16084, VX765, VX799, WIN72052, XIAP, YM215438, Z-AEVD-FMK, Z-DEVD-FMK, Z-FA-FMK, Z-IETD-FMK, Z-LEHD-FMK, Z-LEVD-FMK, Z-VAD-FMK, Z-VDVAD-FMK, Z-VEID-FMK, Z-WEHD-FMK, Z-YVAD-FMK, and analogs thereof. Both specific and broad-spectrum caspase inhibitors may be used. Additional caspase inhibitors are described in U.S. Pat. Nos. 7,074,782, 7,053,057, 7,026,472, 6,921,765, 6,800,619, 6,737,511, 6,716,818, 6,703,500, 6,689,784, 6,632,962, 6,620,782, 6,566,338, 6,559,304, 6,495,522, 6,368,831, 6,355,618, 6,352,844, and 6,153,591.

Flavonoids/Antioxidants

Flavonoids and other antioxidants are chemical compounds that can bind to free oxygen radicals, preventing these radicals from damaging healthy cells. Neurodegenerative disorders are often caused by or accompanied by oxidative stress, and thus flavonoids and other antioxidants may be useful in the present invention to treat neurodegenerative disorders. Flavonoids and other antioxidants that may be suitable for use in the compositions, kits, and methods of the invention include, e.g., flavonols (e.g., myricetin and quercetin), flavones (e.g., apigenin and luteolin), flavanones (e.g., hesperetin and naringenin), flavan-3-ols (e.g., catechin, epicatechin, epicatechin gallate, epigallocatechin, and epigallocatechin gallate), anthocyanidins (e.g., cyanidin, delphinidin, malvidin, pelargonidin, and peonidin), vitamin A, vitamin C, vitamin E, lycopene, and beta-carotene. Additional flavonoids and other antioxidants are described in Ramassamy, Eur. J. Pharmacol. 545:51-64, 2006, Kang et al., Bioorg. Med. Chem. Lett. 15:3588-3591, 2005, Simonyi et al., Mol. Neurobiol. 31:135-147, 2005, Aruoma et al., Mutat. Res. 544:203-215, 2003, Youdim et al., FASEB J. 17:1943-1944, 2003, and Sloley et al., J. Pharm. Pharmacol. 52:451-459, 2000.

PKR Inhibitors

PKR (also referred to as RNA-dependent protein kinase) is involved in a variety of cellular processes, including signal transduction, differentiation, and apoptosis. Inhibitors of PKR may be used in the present invention to treat disorders associated with abnormal cellular responses, e.g., neurodegenerative disorders. PKR inhibitors that may be suitable for use in the compositions, kits, and methods of the invention include, e.g., those described in Jammi et al., Biochem. Biophys. Res. Commun. 308:50-57, 2003 (Calbiochem Cat. No. 527450), Shimazawa et al., Neurosci. Lett. 409:192-195, 2006, Peel, J. Neuropathol. Exp. Neurol. 63:97-105, 2004, Bando et al., Neurochem. Int. 46:11-18, 2005, Peel et al., Hum. Mol. Genet. 10:1531-1538, 2001, and Chang et al., J. Neurochem. 83:1215-1225, 2002.

Additional Therapeutic Regimens

If desired, the patient may also receive additional therapeutic regimens. For example, therapeutic agents may be administered with the agent or agents described herein at concentrations known to be effective for such therapeutic agents. Agents that may be particularly useful include those that prevent or slow the rate of neural deterioration or death, or those that treat, prevent, or ameliorate one or more symptoms of a neurodegenerative disorder. Exemplary therapeutic classes and agents are listed in Table 2. Combinations of the classes or agents of Table 2 may also be used.

If more than one agent is employed, therapeutic agents may be delivered separately or may be admixed into a single formulation. When agents are present in different pharmaceutical compositions, different routes of administration may be employed. Routes of administration for the various embodiments include, but are not limited to, topical, transdermal, and systemic administration (e.g., intravenous, intramuscular, subcutaneous, inhalation, rectal, buccal, vaginal, intraperitoneal, intraarticular, ophthalmic or oral administration). Alternatively, agents may be administered by intracranial, intrathecal, or epidural administration. Any method of administration that bypasses the blood-brain barrier or enhances its permeability (e.g., administration of a Na⁺/Ca⁺⁺ exchange blocker, mannitol, or Cereport) may be useful in the invention.

In some instances, the agent of the invention and additional therapeutic agents are administered at least one hour, two hours, four hours, six hours, 10 hours, 12 hours, 18 hours, 24 hours, three days, seven days, or 14 days apart. The dosage and frequency of administration of each component of the combination can be controlled independently. For example, one compound may be administered three times per day, while the second compound may be administered once per day. Combination therapy may be given in on-and-off cycles that include rest periods so that the patient's body has a chance to recover from any as yet unforeseen side effects. The compounds may also be formulated together such that one administration delivers both compounds. Optionally, any of the agents of the combination may be administered in a low dosage or in a high dosage, each of which is defined herein.

The therapeutic agents of the invention may be admixed with additional active or inert ingredients, e.g., in conventional pharmaceutically acceptable carriers. A pharmaceutical carrier can be any compatible, non-toxic substance suitable for the administration of the compositions of the present invention to a patient. Pharmaceutically acceptable carriers include, for example, water, saline, buffers and other compounds, described, for example, in the Merck Index, Merck & Co., Rahway, N.J. Slow release formulation or a slow release apparatus may be also be used for continuous administration.

In addition to the administration of therapeutic agents, the additional therapeutic regimen may involve other therapies, e.g., transplantation of neural cells (including, if needed, anti-inflammatory and/or immunosuppressive therapy), or a modification to the lifestyle of the patient being treated.

Conjugates

If desired, the drugs used in any of the combinations described herein may be covalently attached to one another to form a conjugate of formula (XI).
(A)-(L)-(B) (XI)

In formula (XI), (A) is an agent listed in Table 1a or 1b covalently tethered via a linker (L) to (B), any agent of the classes or agents listed in Tables 1a, 1b, and 2.

Conjugates of the invention can be administered to a subject by any route and for the treatment of any disease described herein.

The conjugates of the invention can be prodrugs, releasing drug (A) and drug (B) upon, for example, cleavage of the conjugate by intracellular and extracellular enzymes (e.g., amidases, esterases, and phosphatases). The conjugates of the invention can also be designed to largely remain intact in vivo, resisting cleavage by intracellular and extracellular enzymes. The degradation of the conjugate in vivo can be controlled by the design of linker (L) and the covalent bonds formed with drug (A) and drug (B) during the synthesis of the conjugate.

Conjugates can be prepared using techniques familiar to those skilled in the art. For example, the conjugates can be prepared using the methods disclosed in G. Hermanson, Bioconjugate Techniques, Academic Press, Inc., 1996. The synthesis of conjugates may involve the selective protection and deprotection of alcohols, amines, ketones, sulfhydryls or carboxyl functional groups of drug (A), the linker, and/or drug (B). For example, commonly used protecting groups for amines include carbamates, such as tert-butyl, benzyl, 2,2,2-trichloroethyl, 2-trimethylsilylethyl, 9-fluorenylmethyl, allyl, and m-nitrophenyl. Other commonly used protecting groups for amines include amides, such as formamides, acetamides, trifluoroacetamides, sulfonamides, trifluoromethanesulfonyl amides, trimethylsilylethanesulfonamides, and tert-butylsulfonyl amides. Examples of commonly used protecting groups for carboxyls include esters, such as methyl, ethyl, tert-butyl, 9-fluorenylmethyl, 2-(trimethylsilyl)ethoxy methyl, benzyl, diphenylmethyl, O-nitrobenzyl, ortho-esters, and halo-esters. Examples of commonly used protecting groups for alcohols include ethers, such as methyl, methoxymethyl, methoxyethoxymethyl, methylthiomethyl, benzyloxymethyl, tetrahydropyranyl, ethoxyethyl, benzyl, 2-napthylmethyl, O-nitrobenzyl, P-nitrobenzyl, P-methoxybenzyl, 9-phenylxanthyl, trityl (including methoxy-trityls), and silyl ethers. Examples of commonly used protecting groups for sulfhydryls include many of the same protecting groups used for hydroxyls. In addition, sulfhydryls can be protected in a reduced form (e.g., as disulfides) or an oxidized form (e.g., as sulfonic acids, sulfonic esters, or sulfonic amides). Protecting groups can be chosen such that selective conditions (e.g., acidic conditions, basic conditions, catalysis by a nucleophile, catalysis by a Lewis acid, or hydrogenation) are required to remove each, exclusive of other protecting groups in a molecule. The conditions required for the addition of protecting groups to amine, alcohol, sulfhydryl, and carboxyl functionalities and the conditions required for their removal are provided in detail in T. W. Green and P. G. M. Wuts, Protective Groups in Organic Synthesis (2^{nd} Ed.), John Wiley & Sons, 1991 and P. J. Kocienski, Protecting Groups, Georg Thieme Verlag, 1994. Additional synthetic details are provided below.

Linkers

The linker component of the invention is, at its simplest, a bond between drug (A) and drug (B), but typically provides a linear, cyclic, or branched molecular skeleton having pendant groups covalently linking drug (A) to drug (B).

Thus, linking of drug (A) to drug (B) is achieved by covalent means, involving bond formation with one or more functional groups located on drug (A) and drug (B). Examples of chemically reactive functional groups which may be employed for this purpose include, without limitation, amino, hydroxyl, sulfhydryl, carboxyl, carbonyl, carbohydrate groups, vicinal diols, thioethers, 2-aminoalcohols, 2-aminothiols, guanidinyl, imidazolyl, and phenolic groups.

The covalent linking of drug (A) and drug (B) may be effected using a linker which contains reactive moieties capable of reaction with such functional groups present in drug (A) and drug (B). For example, an amine group of drug (A) may react with a carboxyl group of the linker, or an activated derivative thereof, resulting in the formation of an amide linking the two.

Examples of moieties capable of reaction with sulfhydryl groups include α-haloacetyl compounds of the type XCH₂CO— (where X═Br, Cl or I), which show particular reactivity for sulfhydryl groups, but which can also be used to modify imidazolyl, thioether, phenol, and amino groups as described by Gurd, Methods Enzymol. 11:532 (1967). N-Maleimide derivatives are also considered selective towards sulfhydryl groups, but may additionally be useful in coupling to amino groups under certain conditions. Reagents such as 2-iminothiolane (Traut et al., Biochemistry 12:3266 (1973)), which introduce a thiol group through conversion of an amino group, may be considered as sulfhydryl reagents if linking occurs through the formation of disulphide bridges.

Examples of reactive moieties capable of reaction with amino groups include, for example, alkylating and acylating agents. Representative alkylating agents include:

(i) α-haloacetyl compounds, which show specificity towards amino groups in the absence of reactive thiol groups and are of the type XCH₂CO— (where X═Cl, Br or I), for example, as described by Wong Biochemistry 24:5337 (1979);

(ii) N-maleimide derivatives, which may react with amino groups either through a Michael type reaction or through acylation by addition to the ring carbonyl group, for example, as described by Smyth et al., J. Am. Chem. Soc. 82:4600 (1960) and Biochem. J. 91:589 (1964);

(iii) aryl halides such as reactive nitrohaloaromatic compounds;

(iv) alkyl halides, as described, for example, by McKenzie et al., J. Protein Chem. 7:581 (1988);

(v) aldehydes and ketones capable of Schiff's base formation with amino groups, the adducts formed usually being stabilized through reduction to give a stable amine;

(vi) epoxide derivatives such as epichlorohydrin and bisoxiranes, which may react with amino, sulfhydryl, or phenolic hydroxyl groups;

(vii) chlorine-containing derivatives of s-triazines, which are very reactive towards nucleophiles such as amino, sufhydryl, and hydroxyl groups;

(viii) aziridines based on s-triazine compounds detailed above, e.g., as described by Ross, J. Adv. Cancer Res. 2:1 (1954), which react with nucleophiles such as amino groups by ring opening;

(ix) squaric acid diethyl esters as described by Tietze, Chem. Ber. 124:1215 (1991); and

(x) α-haloalkyl ethers, which are more reactive alkylating agents than normal alkyl halides because of the activation caused by the ether oxygen atom, as described by Benneche et al., Eur. J. Med. Chem. 28:463 (1993).

Representative amino-reactive acylating agents include:

(i) isocyanates and isothiocyanates, particularly aromatic derivatives, which form stable urea and thiourea derivatives respectively;

(ii) sulfonyl chlorides, which have been described by Herzig et al., Biopolymers 2:349 (1964);

(iii) acid halides;

(iv) active esters such as nitrophenylesters or N-hydroxysuccinimidyl esters;

(v) acid anhydrides such as mixed, symmetrical, or N-carboxyanhydrides;

(vi) other useful reagents for amide bond formation, for example, as described by M. Bodansky, Principles of Peptide Synthesis, Springer-Verlag, 1984;

(vii) acylazides, e.g. wherein the azide group is generated from a preformed hydrazide derivative using sodium nitrite, as described by Wetz et al., Anal. Biochem. 58:347 (1974); and

(viii) imidoesters, which form stable amidines on reaction with amino groups, for example, as described by Hunter and Ludwig, J. Am. Chem. Soc. 84:3491 (1962).

Aldehydes and ketones may be reacted with amines to form Schiff's bases, which may advantageously be stabilized through reductive amination. Alkoxylamino moieties readily react with ketones and aldehydes to produce stable alkoxamines, for example, as described by Webb et al., in Bioconjugate Chem. 1:96 (1990).

Examples of reactive moieties capable of reaction with carboxyl groups include diazo compounds such as diazoacetate esters and diazoacetamides, which react with high specificity to generate ester groups, for example, as described by Herriot, Adv. Protein Chem. 3:169 (1947). Carboxyl modifying reagents such as carbodiimides, which react through O-acylurea formation followed by amide bond formation, may also be employed.

It will be appreciated that functional groups in drug (A) and/or drug (B) may, if desired, be converted to other functional groups prior to reaction, for example, to confer additional reactivity or selectivity. Examples of methods useful for this purpose include conversion of amines to carboxyls using reagents such as dicarboxylic anhydrides; conversion of amines to thiols using reagents such as N-acetylhomocysteine thiolactone, S-acetylmercaptosuccinic anhydride, 2-iminothiolane, or thiol-containing succinimidyl derivatives; conversion of thiols to carboxyls using reagents such as α-haloacetates; conversion of thiols to amines using reagents such as ethylenimine or 2-bromoethylamine; conversion of carboxyls to amines using reagents such as carbodiimides followed by diamines; and conversion of alcohols to thiols using reagents such as tosyl chloride followed by transesterification with thioacetate and hydrolysis to the thiol with sodium acetate.

So-called zero-length linkers, involving direct covalent joining of a reactive chemical group of drug (A) with a reactive chemical group of drug (B) without introducing additional linking material may, if desired, be used in accordance with the invention.

Most commonly, however, the linker will include two or more reactive moieties, as described above, connected by a spacer element. The presence of such a spacer permits bifunctional linkers to react with specific functional groups within drug (A) and drug (B), resulting in a covalent linkage between the two. The reactive moieties in a linker may be the same (homobifunctional linker) or different (heterobifunctional linker, or, where several dissimilar reactive moieties are present, heteromultifunctional linker), providing a diversity of potential reagents that may bring about covalent attachment between drug (A) and drug (B).

Spacer elements in the linker typically consist of linear or branched chains and may include a C₁-C₁₀ alkyl, C₂-C₁₀ alkenyl, C₂-C₁₀ alkynyl, C₂-C₆ heterocyclyl, C₆-C₁₂ aryl, C₇-C₁₄ alkaryl, C₃-C₁₀ alkheterocyclyl, or C₁-C₁₀ heteroalkyl.

In some instances, the linker is described by formula (XII):
G¹-(Z¹)ₒ-(Y¹)ᵤ-(Z²)ₛ-(R₃₀)-(Z³)ₜ-(Y²)ᵥ-(Z⁴)ₚ-G² (XII)

In formula (XII), G¹ is a bond between drug (A) and the linker; G² is a bond between the linker and drug (B); Z¹, Z², Z³, and Z⁴ each, independently, is selected from O, S, and NR₃₁; R₃₁ is hydrogen, C₁-C₆ alkyl, C₂-C₆ alkenyl, C₂-C₆ alkynyl, C₂-C₆ heterocyclyl, C₆-C₁₂ aryl, C₇-C₁₄ alkaryl, C₃-C₁₀ alkheterocyclyl, or C₁-C₇ heteroalkyl; Y¹ and Y² are each, independently, selected from carbonyl, thiocarbonyl, sulphonyl, or phosphoryl; o, p, s, t, u, and v are each, independently, 0 or 1; and R₃₀ is a C₁-C₁₀ alkyl, C₂-C₁₀ alkenyl, C₂-C₁₀ alkynyl, C₂-C₆ heterocyclyl, C₆-C₁₂ aryl, C₇-C₁₄ alkaryl, C₃-C₁₀ alkheterocyclyl, or C₁-C₁₀ heteroalkyl, or a chemical bond linking G¹-(Z¹)ₒ-(Y¹)ᵤ-(Z²)ₛ- to -(Z³)ₜ-(Y²)ᵥ-(Z⁴)ₚ-G².

Examples of homobifunctional linkers useful in the preparation of conjugates of the invention include, without limitation, diamines and diols selected from ethylenediamine, propylenediamine and hexamethylenediamine, ethylene glycol, diethylene glycol, propylene glycol, 1,4-butanediol, 1,6-hexanediol, cyclohexanediol, and polycaprolactone diol.

Formulation

Any of the agents employed according to the present invention may be contained in any appropriate amount in any suitable carrier substance, and is generally present in an amount of 1-95% by weight of the total weight of the composition. The composition may be provided in a dosage form that is suitable for the oral, parenteral (e.g., intravenously, intramuscularly), rectal, cutaneous, nasal, vaginal, inhalant, skin (patch), or ocular administration route. Thus, the composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, or aerosols. The pharmaceutical compositions may be formulated according to conventional pharmaceutical practice (see, e.g., Remington: The Science and Practice of Pharmacy, 20th edition, 2000, ed. A. R. Gennaro, Lippincott Williams & Wilkins, Philadelphia, and Encyclopedia of Pharmaceutical Technology, eds. J. Swarbrick and J. C. Boylan, 1988-1999, Marcel Dekker, New York).

If more than one agent is employed, each agent may be formulated in a variety of ways that are known in the art. Desirably, the agents are formulated together for the simultaneous or near simultaneous administration of the agents. Such co-formulated compositions can include the two agents formulated together in the same pill, capsule, liquid, etc. It is to be understood that, when referring to the formulation of such combinations, the formulation technology employed is also useful for the formulation of the individual agents of the combination, as well as other combinations of the invention. By using different formulation strategies for different agents, the pharmacokinetic profiles for each agent can be suitably matched.

The individually or separately formulated agents can be packaged together as a kit. Non-limiting examples include kits that contain, e.g., two pills, a pill and a powder, a suppository and a liquid in a vial, two topical creams, etc. The kit can include optional components that aid in the administration of the unit dose to patients, such as vials for reconstituting powder forms, syringes for injection, customized IV delivery systems, inhalers, etc. Additionally, the unit dose kit can contain instructions for preparation and administration of the compositions. The kit may be manufactured as a single use unit dose for one patient, multiple uses for a particular patient (at a constant dose or in which the individual compounds may vary in potency as therapy progresses); or the kit may contain multiple doses suitable for administration to multiple patients (“bulk packaging”). The kit components may be assembled in cartons, blister packs, bottles, tubes, and the like.

Formulations and Methods for Delivery of Agents to the Brain

Treatment of neurodegenerative disorders, e.g., HD, may be hampered by the inability of an active, therapeutic compound to cross the blood-brain barrier (BBB). Strategies for delivery of compositions of the invention to the brain include strategies to bypass the BBB (e.g., intracranial administration via craniotomy and intrathecal administration), and strategies to cross the BBB (e.g., the use of compounds that increase permeability of the BBB in conjunction with systemic administration of compositions of the invention, and modification of compositions of the invention to increase their permeability or transport across the BBB).

Craniotomy, a procedure known in the art, can be used with any composition of the invention for delivery to the brain. In this approach, an opening is made in the patient's cranium, and a compound is delivered via a catheter. This approach can be used to target a compound to a specific area of the brain.

Intrathecal administration provides another means of bypassing the BBB for drug delivery. Briefly, drugs are administered to the spinal chord, for example, via lumbar puncture or through the use of devices such as pumps. Lumbar puncture is preferable for single or infrequent administration, whereas constant and/or chronic administration may be achieved using any commercially available pump attached to a intraspinal catheter, e.g., a pump and catheter made by Medtronic (Minneapolis, Minn.).

To allow for delivery across the BBB, compositions of the invention can be administered along with a compound or compounds that induce a transient increase in permeability of the BBB. Such compounds include mannitol, Cereport (RMP-7), and KB-R7943, a Na⁺/Ca⁺⁺ exchange blocker.

Compounds of the invention can be modified (e.g., lipidated or acetylated) to increase transport across the BBB following systemic administration (e.g., parenteral) by using chemical modifications that are standard in the art. In one embodiment, compounds of the invention are conjugated to peptide vectors that are transported across the BBB. For example, compounds may be conjugated to a monoclonal antibody to the human insulin receptor as described by Partridge (Jpn. J. Pharmacol. 87:97-103, 2001), thus permitting the compound to be transported across the BBB following systemic administration. Compounds of the invention can be conjugated to such peptide vectors, for example, using biotin-streptavidin technology.

Dosages

Generally, when administered to a human, the dosage of any of the agents of the combination of the invention will depend on the nature of the agent, and can readily be determined by one skilled in the art. Typically, such dosage is normally about 0.001 mg to 2,000 mg per day, about 1 mg to 1,000 mg per day, or about 5 mg to 500 mg per day.

Administration of each drug in the combination can, independently, be one to four times daily for one day to one year, and may even be for the life of the patient. Chronic, long-term administration will be indicated in many cases.

Additional Applications

If desired, the compounds of the invention may be employed in mechanistic assays to determine whether other combinations, or single agents, are as effective as the combination in treating, preventing, or ameliorating neurodegenerative disorders (e.g., HD or any of its associated conditions) using assays generally known in the art, examples of which are described herein. For example, candidate compounds may be tested, alone or in combination with one or more compounds selected independently from any of the agents of Tables 1a and 1b, and applied to cells, e.g., neural cells or PC12 cells, expressing a toxic mutant polyglutamine repeat protein. After a suitable time, these cells are examined for viability. An increase in viability, in comparison to control cells not treated with the candidate compound, identifies a candidate compound or combination of agents as an effective agent to treat, prevent, or ameliorate a neurodegenerative disorder.

The agents of the invention may also be useful tools in elucidating mechanistic information about the biological pathways involved in neural cell deterioration and death. Such information can lead to the development of new combinations or single agents for treating, preventing, or ameliorating neurodegenerative disorders. Methods known in the art to determine biological pathways can be used to determine the pathway, or network of pathways, affected by contacting cells, e.g., neural cells, with the compounds of the invention. Such methods can include analyzing cellular constituents that are expressed or repressed after contact with the compounds of the invention as compared to untreated, positive or negative control compounds, and/or new single agents and combinations, or analyzing some other activity of the cell such as enzyme activity, nutrient uptake, and proliferation. Cellular components analyzed can include gene transcripts and protein expression. Suitable methods can include standard biochemistry techniques, radiolabeling the compounds of the invention (e.g., ¹⁴C or ³H labeling), and observing the compounds binding to proteins, e.g. using 2D gels, gene expression profiling. Once identified, such compounds can be used in in vivo models to further validate the tool or develop new agents or strategies to treat, prevent, or ameliorate neurodegenerative disorders.

As indicated above, the methods of this invention may also be used prophylactically, in patients who are at an increased risk of developing a neurodegenerative disorder, e.g., HD, or a condition associated with such a disorder. Risk factors include, for example, age, family history of neurodegenerative disorders, and psychological or psychiatric profile.

Exemplary Candidate Compounds

Peptide Moieties

Peptides, peptide mimetics, and peptide fragments (whether natural, synthetic or chemically modified) may be suitable for use in practicing the invention. Exemplary compounds include those that reduce the amount of target protein or RNA levels (e.g., antisense compounds, dsRNA, ribozymes) and compounds that compete with endogenous mitotic kinesins or protein tyrosine phosphatases for binding partners (e.g., dominant negative proteins or polynucleotides encoding the same).

Antisense Compounds

The biological activity of any protein that increases cell death, e.g., mutant Htt, can be reduced through the use of an antisense compound directed to RNA encoding the target protein. Antisense compounds that reduce expression of target molecules can be identified using standard techniques. For example, accessible regions of the mRNA of the target enzyme can be predicted using an RNA secondary structure folding program such as MFOLD (M. Zuker, D. H. Mathews & D. H. Turner, Algorithms and Thermodynamics for RNA Secondary Structure Prediction: A Practical Guide. In: RNA Biochemistry and Biotechnology, J. Barciszewski & B. F. C. Clark, eds., NATO ASI Series, Kluwer Academic Publishers, (1999)). Sub-optimal folds with a free energy value within 5% of the predicted most stable fold of the mRNA are predicted using a window of 200 bases within which a residue can find a complimentary base to form a base pair bond. Open regions that do not form a base pair are summed together with each suboptimal fold and areas that are predicted as open are considered more accessible to the binding to antisense nucleobase oligomers. Other methods for antisense design are described, for example, in U.S. Pat. No. 6,472,521, Antisense Nucleic Acid Drug Dev. 1997 7:439-444, Nucleic Acids Research 28:2597-2604, 2000, and Nucleic Acids Research 31:4989-4994, 2003.

RNA Interference

The biological activity of a target molecule can be reduced through the use of RNA interference (RNAi), employing, e.g., a double stranded RNA (dsRNA) or small interfering RNA (siRNA) directed to the target molecule in question (see, e.g., Miyamoto et al., Prog. Cell Cycle Res. 5:349-360, 2003; U.S. Patent Application Publication No. 20030157030). Methods for designing such interfering RNAs are known in the art. For example, software for designing interfering RNA is available from Oligoengine (Seattle, Wash.).

Dominant Negative Proteins

One skilled in the art would know how to make dominant negative proteins to the target molecules to be targeted. Such dominant negative proteins are described, for example, in Gupta et al., J. Exp. Med., 186:473-478, 1997; Maegawa et al., J. Biol. Chem. 274:30236-30243, 1999; Woodford-Thomas et al., J. Cell Biol. 117:401-414, 1992).

The following example is provided for the purpose of illustrating the invention and is not meant to limit the invention in any way.

### EXAMPLE

### Screening Assays

The present invention provides screening methods for identifying candidate compounds that treat, prevent, or ameliorate neurodegenerative disorders, e.g., HD.

A variety of model systems, including cellular as well as animal models, have demonstrated that the exon 1 portion of Htt, containing an expanded polyglutamine region, is sufficient to cause pathology. For example, the N-terminal fragment of Htt has been shown to form protein aggregates in the nucleus, cytoplasm and processes of neurons in human HD patients and in HD animal models, as well as in many cellular models.

Because of their similarities to neurons, rat pheochromocytoma PC12 cells have provided a useful model for studying neuronal cell biology. In addition, PC12 cells are readily transfected, selected and cloned. Both before and after terminal differentiation with NGF, PC12 cells exhibit many characteristics of mature neurons, including the ability to undergo growth factor withdrawal-induced apoptotic cell death.

In order to perform screening according to a method of the present invention, PC12 cells were obtained that stably incorporated a plasmid that inducibly expresses a toxic expanded polyglutamine (103 glutamine) form of exon 1 of Htt, fused to the marker EGFP. In this stable PC12 line (PC12/HttN90Q103), transcription of the Htt transgene is driven by an ecdysone-regulated promoter such that the expression of HttN90Q103 can be turned on or off by the addition or removal of the non-steroidal ecdysone analog tebufenozide (teb).

Using the engineered PC12/HttN90Q103 cell line together with Perkin Elmer's ATPlite™ assay kit or CellTiter-Blue™ assay, a high throughput assay to screen small molecules for their ability to prevent mutant Htt exon 1-induced cell death was developed and optimized.

The goal of the assay was to identify single agents as well as combinations of agents that rescue HttN90Q103-induced cell death in the PC12 cell line. Assay performance was evaluated by looking at a variety of parameters, e.g., Z′ factors (described below), plate-to-plate variations, and performance of the positive control compound BOC-D-FMK (20 μM).

The following formulas were used to calculate mutant Htt-induced cytotoxicity and test compound rescue:
% killing=[(un-induced−induced-DMSO treated)/un-induced]×100
% rescue=[(drug treated−induced-DMSO treated)/(un-induced−induced-DMSO treated)]×100

More than 50% of killing was consistently achieved 72 hours following tebufenozide induction when comparing the un-induced wells with DMSO-treated induced wells. In addition, over 50% rescue was reproducibly achieved by 20 μM BOC-D-FMK, providing confirmation that teb induction is in fact effective.

For single-agent ranking, the assay was performed in 384-well plates to obtain duplicate dose response curves in 12-step dilutions with a dosing ratio f=2 over 3 orders of magnitude.

Single agent activity was characterized by fitting a sigmoidal function of the form I=IₘₐₓC^{α}/[C^{α}+EC₅₀^{α}], with least squares minimization using a downhill simplex algorithm (C is the concentration, EC₅₀ is the agent concentration required to obtain 50% of the maximum effect, and α is the sigmoidicity). The uncertainty of each fitted parameter was estimated from the range over which the change in reduced chi-squared was less than one, or less than minimum reduced chi-squared if that minimum exceeded one, to allow for underestimated σ_{I} errors.

Single agent curve data were used to define a dilution series for each compound to be used for combination screening in a 6×6 matrix format. Using a dilution factor f of 2, 3, or 4, depending on the sigmoidicity of the single agent curve, five dose levels were chosen with the central concentration close to the fitted EC₅₀. For compounds with no detectable single agent activity, a dilution factor of 4 was used, starting from the highest achievable concentration.

The Loewe additivity model was used to quantify combination effects. Combinations were ranked initially by Additivity Excess Volume, which is defined as ADD Volume=ΣC_{X}, C_{Y} (I_{data}−I_{Loewe}). where I_{Loewe}(C_{X},C_{Y}) is the inhibition that satisfies (C_{X}/EC_{X})+(C_{Y}/EC_{Y})=1, and EC_{X,Y} are the effective concentrations at I_{Loewe} for the single agent curves. A “Synergy Score” was also used, where the Synergy Score S=log f_{X} log f_{Y} Σ I_{data} (I_{data}−I_{Loewe}), summed over all non-single-agent concentration pairs, and where log f_{X,Y} is the natural logarithm of the dilution factors used for each single agent. This effectively calculates a volume between the measured and Loewe additive response surfaces, weighted towards high inhibition and corrected for varying dilution factors. An uncertainty σ_{S} was calculated for each synergy score, based on the measured errors for the I_{data} values and standard error propagation.

To select desirable combinations for follow-up characterization, the following criteria were established: (1) significant synergy over the additive model as measured by the ADD Volume with a cut-off value=0.4; (2) substantial activity where the synergy occurs and/or sufficient potency shifting, with a maximum effect greater or equal to 30% for combinations.

Based upon the combination screen, the combination agents listed in Table 3 were identified. As indicated by the ADD Volume, combination of the two agents provides a greater degree of rescue of cell death than would be expected based on the rescue by each agent of the combination individually.

After plates were read, the raw data were analyzed, and automated quality control criteria were used to assess the quality of the data from each plate based on the control data contained in the plate. The automated analysis first determined plates to be verified, rejected or undetermined. All plates were then evaluated manually on a plate-by-plate basis and, if necessary, assigned a status of hand accepted or rejected. Additionally, individual blocks of data on verified plates could be manually marked for exclusion.

The quality control criterion for automated analysis was called the Z′-factor, which is defined as:

$${Z}^{′} = 1 - \frac{3 SD of DMSO + 3 SD of control}{\vert median DMSO - median of control \vert}$$

Here, SD represents standard deviation, and DMSO represents teb-induced and vehicle treated. To assess the assay performance, two forms of Z′ factors were calculated using two types of controls:
Control defined by the wells treated with 20 μM BOC-D-FMK on each plate
Control defined by un-induced wells on each plate

Based on the Z′-factor, automated quality control marked a plate as verified (Z′>0.6), rejected (Z′<0.4 or Z′>1), or undetermined (0.4<Z′<0.6). Plates that were rejected either automatically or by visual inspection were excluded from further analysis and were scheduled to be repeated.

In addition to manually verifying plates with marginal Z′-factors, all plates were visually inspected for occasional bad wells, or “spikes.” Individual wells with data values that were very different from their immediate neighbors (within the same treatment class) were flagged and excluded from subsequent analyses. Plates containing an unusually large number of spikes were rejected altogether.

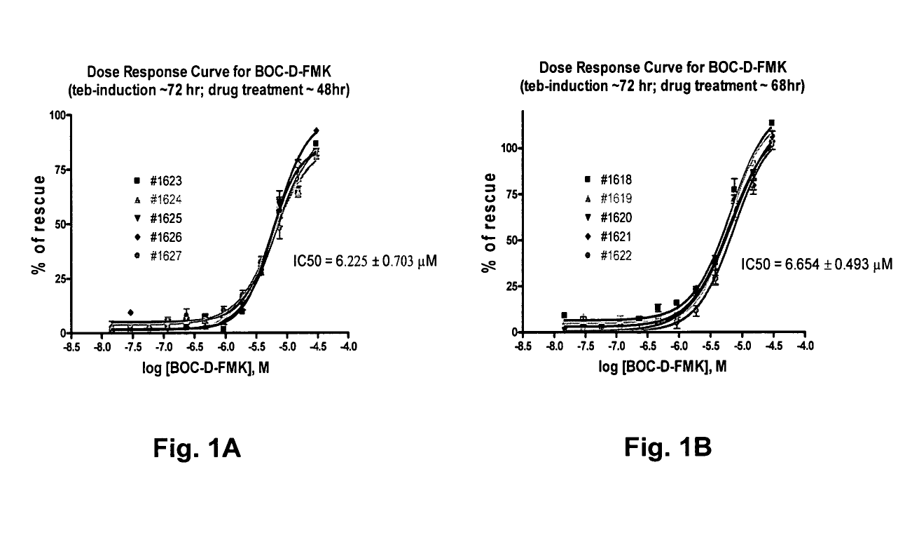

FIGS. 1A-1B show ten dose response curves generated for the positive control BOC-D-FMK during the same test run, one curve from each of the ranking plates. The data show that the ATPlite™ assay using PC12 cells performed quite well with excellent Z′ scores (between 0.6 and 0.8) with very small plate-to-plate variations as indicated by the IC₅₀ values obtained from each of the replicate plates.

Hits from the ranking experiments are shown in Tables 1a and 1b.

Detailed protocols for the ATPlite™ and CellTiter-Blue™ assays follow.

Protocol for ATPlite™ Assay Using PC12/HttN90Q103 Cells Day 1—Seed Cells and Add Tebufenozide Inducer
1) Make a complete medium:
   For PC12/httN90Q103 and Q25: DMEM containing 5% FBS, 5% HS, 1% penicillin/streptomycin, 1% L-glutamine, 25 mM HEPES (GIBCO), and 0.5 mg/mL G418 (GIBCO)
2) Warm up medium and 0.25% trypsin-EDTA to 37° C. before seeding cells.
3) Aspirate old medium from the T175 flask.
4) Add 2 mL 0.25% trypsin-EDTA to the T175 flask and allow to sit for two minutes.
5) Add 8 mL of the medium to the flask to inactivate trypsin.
6) Pipette cells several times to make sure that cells are well separated.
7) Count cells using a hemacytometer.
8) Make up final cell solution at a density of 200,000 cells/mL and seed 30 L (6,000) cells/well into 384-well plates.
9) Add 10 μL media to wells in column 24 (uninduced control).
10) Make up 1 μM tebufenozide solution: dilute 1 mM stock 1:1000 in media.
11) Add tebufenozide inducer (10 μL per well) using multidrop to wells in columns 1-23 to a final concentration of 250 nM.

Day 1—Add Compounds 4 hours post-induction
1) Remove compound plates from dessicator.
2) Using the PlateMate, prepare dilution plate with 100 μL per well of the complete cell medium at room temperature in clear untreated 384-well plates.
3) Using the MiniTrak, make compound dilution plates: 1 μL from each well of stock plates into 100 μL of media in the dilution plate for a 1:100 dilution (10× stock in media).
4) Using the MiniTrak, add 4.5 μL of the diluted stock to each well of cells in assay plates.
5) Make positive control solution: dilute 20 mM stock of BOC-D-FMK in media for a 10× stock.
6) Manually add 4.5 μL positive control to C1-N1.
7) Incubate plates at 37° C. for three days.
(for ranking combinations, steps 5 and 6 are omitted as there is positive control on the compound plates)

Day 4—Perform ATPlite™ Assay
1) Take plates out of the incubator.
2) Using the PlateMate, add 40 μL of ATPlite™ 1-step solution (Perkin Elmer) to each well of the assay plates.
3) Wait ten minutes to allow plates to dark-adapt.
4) Read luminescence intensity in Wallac plate reader using

Or

Day 4—CellTiter-Blue™ assay
1) Make solution of 5% CellTiter-Blue™ in Q103 media.
2) Using the Multidrop, add 40 μL of 5% CellTiter-Blue™ solution to each well of the assay plates.
3) Incubate plates with CellTiter-Blue™ at 37° C. (9.5% CO2) for 4 hours.
4) Read fluorescence intensity in Wallac plate reader using Alamar Blue program.

The screening methods of the invention described herein may be varied. For example, any cell line expressing a CAG repeat gene containing an expanded CAG repeat region may be used. Screening assays directed to a given polyglutamine repeat disorder may be varied, e.g., by utilizing a cell line expressing a polyglutamine repeat protein, or fragment thereof, associated with that disorder. Any cutoff for hit picking may be chosen, e.g., 1%, 2%, 5%, 10%, 15%, 20%, 30%, 40%, 50%, 60%, 70%, 80%, or 90%. In addition, any method of assaying cell viability may be employed.

### OTHER EMBODIMENTS

All publications, patents, and patent applications mentioned in the above specification are hereby incorporated by reference. Various modifications and variations of the described method and system of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Other embodiments are in the claims.

What is claimed is:

1. A composition comprising:
(a) a first agent selected from any one of the agents of Tables 1a and 1b; and
(b) a second, different agent selected from any one of the classes or agents of Tables 1a, 1b, and 2.

2. The composition of claim 1, wherein said first agent and said second agent are selected from a single row of Table 3.

3. The composition of claim 1, wherein said first agent and said second agent are present in amounts that, when administered to a patient, are sufficient to treat, prevent, or ameliorate a neurodegenerative disorder.

4. The composition of claim 3, wherein said neurodegenerative disorder is a polyglutamine expansion disorder.

5. The composition of claim 4, wherein said polyglutamine expansion disorder is Huntington's disease.

6. The composition of claim 1, wherein said composition is formulated for oral administration.

7. The composition of claim 1, wherein said composition is formulated for systemic administration.

8. The composition of claim 1, wherein said composition is formulated for intracranial, intrathecal, or epidural administration.

9. A method for treating, preventing, or ameliorating a neurodegenerative disorder, said method comprising administering to a patient a first agent selected from any one of the agents of Table 1a in an amount sufficient to treat, prevent, or ameliorate said neurodegenerative disorder.

10. A method for treating, preventing, or ameliorating a neurodegenerative disorder, said method comprising administering to a patient a first agent selected from any one of the agents of Table 1b, and a second, different agent selected from any one of the classes or agents of Table 2, in amounts sufficient to treat, prevent, or ameliorate said neurodegenerative disorder.

11. A method for treating, preventing, or ameliorating a neurodegenerative disorder, said method comprising administering to a patient at least two different agents selected independently from any of the agents of Tables 1a and 1b, wherein the first and second agents are administered simultaneously or within 28 days of each other, in amounts that together are sufficient to treat, prevent, or ameliorate said neurodegenerative disorder.

12. The method of claim 11, wherein said first agent and said second agent are selected from a single row of Table 3.

13. The method of claim 11, wherein said first and second agents are administered within 14 days of each other.

14. The method of claim 13, wherein said first and second agents are administered within 7 days of each other.

15. The method of claim 14, wherein said first and second agents are administered within 24 hours of each other.

16. The method of claim 11, wherein said neurodegenerative disorder is a polyglutamine expansion disorder.

17. The method of claim 16, wherein said polyglutamine expansion disorder is Huntington's disease.

18. The method of claim 11, wherein said patient is a human.

19. The method of claim 11, further comprising an additional therapeutic regimen.

20. The method of claim 19, wherein said additional therapeutic regimen comprises administering to said patient an additional therapeutic agent, wherein said first agent and said additional therapeutic agent are present in amounts that, when administered to said patient, are sufficient to treat, prevent, or ameliorate a neurodegenerative disorder.

21. The method of claim 20, wherein said additional therapeutic agent is selected from any one of the classes or agents of Table 2.

22. The method of claim 20, wherein said first agent and said additional therapeutic agent are administered within 14 days of each other.

23. The method of claim 22, wherein said first agent and said additional therapeutic agent are administered within 7 days of each other.

24. The method of claim 23, wherein said first agent and said additional therapeutic agent are administered within 24 hours of each other.

25. The method of claim 11, wherein said first agent or said additional therapeutic agent are administered orally.

26. The method of claim 11, wherein said first agent or said additional therapeutic agent are administered systemically.

27. The method of claim 11, wherein said first agent or said additional therapeutic agent are administered intracranially, intrathecally, or epidurally.

28. A method of identifying a combination that may be useful for the treatment, prevention, or amelioration of a neurodegenerative disorder, said method comprising the steps of:
(a) providing cells comprising a gene encoding a polyglutamine repeat polypeptide, wherein said polypeptide comprises an expanded polyglutamine repeat region relative to a wild-type polyglutamine repeat polypeptide;
(b) inducing expression of said gene;
(c) contacting said cells with an agent selected from any one of the agents of Tables 1a and 1b and a candidate compound; and
(d) determining whether the combination of said agent and said candidate compound reduces cell death relative to cells contacted with said agent but not contacted with the candidate compound, wherein a reduction in cell death identifies the combination as a combination useful for the treatment, prevention, or amelioration of a neurodegenerative disorder.

29. The method of claim 28, wherein said polyglutamine repeat polypeptide comprising said expanded polyglutamine repeat region comprises HttN90Q103.

30. The method of claim 28, wherein said cell death is determined by monitoring intracellular ATP levels.

31. The method of claim 28, wherein said cells are mammalian cells.

32. The method of claim 31, wherein said cells are rat pheochromocytoma PC12 cells.
